SEQUENCE LISTING (136 sequences)

SEQ ID NO: 1 - protein, 490 aa, Homo sapiens
MLLVTSLLLCELPHPAFLLIPEVQLVQSGAEVKKPGSSVKVSCKASGGTFSSYAISWVRQAPGQGLEWMGGIIPIFGTANYAQKFQGRVTITADKSTSTAYMELSSLRSEDTAVYYCATFALFGFREQAFDIWGQGTTVTVSSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDLATYYCQQSYSTPFTFGPGTKVDIKSGTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 2 - protein, 731 aa, Homo sapiens
MLLVTSLLLCELPHPAFLLIPEVQLVQSGAEVKKPGSSVKVSCKASGGTFSSYAISWVRQAPGQGLEWMGGIIPIFGTANYAQKFQGRVTITADKSTSTAYMELSSLRSEDTAVYYCATFALFGFREQAFDIWGQGTTVTVSSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDLATYYCQQSYSTPFTFGPGTKVDIKSGLEDPAEPKSPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 3 - skipped

SEQ ID NO: 4 - protein, 20 aa, Homo sapiens
LLVTSLLLCELPHPAFLLIP

SEQ ID NO: 5 - protein, 25 aa, Homo sapiens
EVQLVQSGAEVKKPGSSVKVSCKAS

SEQ ID NO: 6 - protein, 10 aa, Homo sapiens
GGTFSSYAIS

SEQ ID NO: 7 - protein, 14 aa, Homo sapiens
WVRQAPGQGLEWMG

SEQ ID NO: 8 - protein, 17 aa, Homo sapiens
GIIPIFGTANYAQKFQG

SEQ ID NO: 9 - protein, 32 aa, Homo sapiens
RVTITADKSTSTAYMELSSLRSEDTAVYYCAT

SEQ ID NO: 10 - protein, 13 aa, Homo sapiens
FALFGFREQAFDI

SEQ ID NO: 11 - protein, 11 aa, Homo sapiens
WGQGTTVTVSS

SEQ ID NO: 12 - protein, 15 aa, Homo sapiens
GGGGSGGGGSGGGGS

SEQ ID NO: 13 - protein, 23 aa, Homo sapiens
DIQMTQSPSSLSASVGDRVTITC

SEQ ID NO: 14 - protein, 11 aa, Homo sapiens
RASQSISSYLN

SEQ ID NO: 15 - protein, 15 aa, Homo sapiens
WYQQKPGKAPKLLIY

SEQ ID NO: 16 - protein, 7 aa, Homo sapiens
AASSLQS

SEQ ID NO: 17 - protein, 32 aa, Homo sapiens
GVPSRFSGSGSGTDFTLTISSLQPEDLATYYC

SEQ ID NO: 18 - protein, 9 aa, Homo sapiens
QQSYSTPFT

SEQ ID NO: 19 - protein, 10 aa, Homo sapiens
FGPGTKVDIK

SEQ ID NO: 20 - skipped

SEQ ID NO: 21 - protein, 4 aa, Homo sapiens
LEDP

SEQ ID NO: 22 - protein, 126 aa, Homo sapiens
AEPKSPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 23 - protein, 107 aa, Homo sapiens
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 4 aa, Homo sapiens
KDPK

SEQ ID NO: 25 - protein, 45 aa, Homo sapiens
TTTPAPRPPTPAPTIASQPLSLRPEACRPAAGGAVHTRGLDFACD

SEQ ID NO: 26 - protein, 24 aa, Homo sapiens
IYIWAPLAGTCGVLLLSLVITLYC

SEQ ID NO: 27 - protein, 42 aa, Homo sapiens
KRGRKKLLYIFKQPFMRPVQTTQEEDGCSCRFPEEEEGGCEL

SEQ ID NO: 28 - protein, 112 aa, Homo sapiens
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 29 - protein, 469 aa, Homo sapiens
EVQLVQSGAEVKKPGSSVKVSCKASGGTFSSYAISWVRQAPGQGLEWMGGIIPIFGTANYAQKFQGRVTITADKSTSTAYMELSSLRSEDTAVYYCATFALFGFREQAFDIWGQGTTVTVSSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDLATYYCQQSYSTPFTFGPGTKVDIKSGTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 30 - protein, 708 aa, Homo sapiens
EVQLVQSGAEVKKPGSSVKVSCKASGGTFSSYAISWVRQAPGQGLEWMGGIIPIFGTANYAQKFQGRVTITADKSTSTAYMELSSLRSEDTAVYYCATFALFGFREQAFDIWGQGTTVTVSSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDLATYYCQQSYSTPFTFGPGTKVDIKSGLEDPAEPKSPDKTHTCPPCPAPELLGGPSVFLFPPKPKDTLMISRTPEVTCVVVDVSHEDPEVKFNWYVDGVEVHNAKTKPREEQYNSRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 31 - protein, 21 aa, Homo sapiens
GSGEGRGSLLTCGDVEENPGP

SEQ ID NO: 32 - protein, 22 aa, Homo sapiens
GSGATNFSLLKQAGDVEENPGP

SEQ ID NO: 33 - protein, 23 aa, Homo sapiens
GSGQCTNYALLKLAGDVESNPGP

SEQ ID NO: 34 - protein, 25 aa, Homo sapiens
GSGVKQTLNFDLLKLAGDVESNPGP

SEQ ID NO: 35 - protein, 4 aa, Homo sapiens
RKRR

SEQ ID NO: 36 - protein, 242 aa, Homo sapiens
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSS

SEQ ID NO: 37 - protein, 242 aa, Homo sapiens
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSS

SEQ ID NO: 38 - protein, 242 aa, Homo sapiens
QVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIK

SEQ ID NO: 39 - protein, 242 aa, Homo sapiens
QVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIK

SEQ ID NO: 40 - protein, 247 aa, Homo sapiens
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSGGGGSQVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSS

SEQ ID NO: 41 - protein, 247 aa, Homo sapiens
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSS

SEQ ID NO: 42 - protein, 247 aa, Homo sapiens
QVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSSGGGGSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIK

SEQ ID NO: 43 - protein, 247 aa, Homo sapiens
QVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSGGGGSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIK

SEQ ID NO: 44 - protein, 247 aa, Homo sapiens
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSGGGGSQVQLQESGPGLVKPSETLSLTCTVSGVSLPDYGVSWIRQPPGKGLEWIGVIWGSETTYYNSSLKSRVTISKDNSKNQVSLKLSSVTAADTAVYYCAKHYYYGGSYAMDYWGQGTLVTVSS

SEQ ID NO: 45 - protein, 247 aa, Homo sapiens
QVQLQESGPGLVKPSETLSLTCTVSGVSLPDYGVSWIRQPPGKGLEWIGVIWGSETTYYNSSLKSRVTISKDNSKNQVSLKLSSVTAADTAVYYCAKHYYYGGSYAMDYWGQGTLVTVSSGGGGSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIK

SEQ ID NO: 46 - protein, 242 aa, Homo sapiens
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYNSSLKSRVTISKDNSKNQVSLKLSSVTAADTAVYYCAKHYYYGGSYAMDYWGQGTLVTVSS

SEQ ID NO: 47 - protein, 242 aa, Homo sapiens
QVQLQESGPGLVKPSETLSLTCTVSGVSLPDYGVSWIRQPPGKGLEWIGVIWGSETTYYNSSLKSRVTISKDNSKNQVSLKLSSVTAADTAVYYCAKHYYYGGSYAMDYWGQGTLVTVSSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIK

SEQ ID NO: 48 - protein, 264 aa, Homo sapiens
MALPVTALLLPLALLLHAARPQVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCARDMNILATVPFDIWGQGTMVTVSSGGGGSGGGGSGGGGSDIQMTQSPSSLSASVGDRVTITCRASQSISTYLNWYQQKPGKAPNLLIYAAFSLQSGVPSRFSGSGSGTDFTLTINSLQPEDFATYYCQQGDSVPLTFGGGTKLEIK

SEQ ID NO: 49 - protein, 264 aa, Homo sapiens
MALPVTALLLPLALLLHAARPQVQLVQSGAEVKKPGASVKVSCKASGYTFTGYYMHWVRQAPGQGLEWMGWINPNSGGTNYAQKFQGRVTLTRDTSISTVYMELSRLRSDDTAVYYCARDMNILATVPFDIWGQGTMVTVSSGGGGSGGGGSGGGGSDIQMTQSPSSLSASVGDRVTITCRASQSISSYLNWYQQKPGKAPKLLIYAASSLQSGVPSRFSGSGSGTDFTLTVNSLQPEDFATYYCQQGDSVPLTFGGGTRLEIK

SEQ ID NO: 50 - protein, 254 aa, Homo sapiens
MALPVTALLLPLALLLHAARPQVQLVQSGAEVKKPGASVKVSCKASGYIFTGYYIHWVRQAPGQGLEWMGWINPNSGGTQGRVTMTRDTMELSGLRSDDNSPAVYYCARDMNILATVPFDIWGQGTLVTVSSGGGGSGGGGSGGGGSDIQLTQSPSSLSASVGDRVTITCRASQSISSYLNWYQQKPGKAPKLLIYAASSLQSGVPSRFSGSGSGTDFTLTVNSLQPEDFATYYCQQGDSVPLTFGGGTKVEIK

SEQ ID NO: 51 - protein, 266 aa, Homo sapiens
MALPVTALLLPLALLLHAARPQVQLQQSGAEVKKSGASVKVSCKASGYTFTDYYMHWLRQAPGQGLEWMGWINPNSGDTNYAQKFQGRVTLTRDTSISTVYMELSRLRSDDTAVYYCARDMNILATVPFDIWGQGTMVTVSSASGGGGSGGRASGGGGSDIQMTQSPSSLSASVGDRVTITCRASQSISSYLNWYQQKPGKAPKLLIYAASSLQSGVPSRFSGSGSGTDFTLTISSLQPEDFATYYCQQGDSVPLTFGGGTKVEIK

SEQ ID NO: 52 - protein, 263 aa, Homo sapiens
MALPVTALLLPLALLLHAARPQVQLVQSGAEVKKPGASVKVSCKASGYTFTSYWMNWVRQAPGQGLEWMGRIDPYDSETHYNQKFKDRVTMTVDKSTSTAYMELSSLRSEDTAVYYCARGNWDDYWGQGTTVTVSSGGGGSGGGGSGGGGSGGGGSDVQLTQSPSFLSAITCRASKSISVGDRSKDLAVTWYQQKPGKAPKLLIYSGSTLQSGVPSRFSGSGSGTEFTLTISSLQPEDFATYYCQQHNKYPYTFGGGTKVEIK

SEQ ID NO: 53 - protein, 263 aa, Homo sapiens
MALPVTALLLPLALLLHAARPQVQLVQSGAEVKKPGASVKVSCKASGYTFTSYWMNWVRQAPGQGLEWMGRIDPYDSETHYNQKFKDRVTMTVDKSTSTAYMELSSLRSEDTAVYYCARGNWDDYWGQGTTVTVSSGGGGSGGGGSGGGGSGGGGSEVVLTQSPATLSLSPGERATLSCRASKSISKDLAWYQQKPGQAPRLLIYSGSTLQSGIPARFSGSGSGTDFTLTISSLEPEDFAVYYCQQHNKYPYTFGGGTKVEIK

SEQ ID NO: 54 - protein, 263 aa, Homo sapiens
MALPVTALLLPLALLLHAARPQVQLVQSGAEVKKPGASVKVSCKASGYTFTSYWMNWVRQAPGQGLEWMGRIDPYDSETHYNQKFKDRVTMTVDKSTSTAYMELSSLRSEDTAVYYCARGNWDDYWGQGTTVTVSSGGGGSGGGGSGGGGSGGGGSDVVMTQSPAFLSVTPGEKVTITCRASKSISKDLAWYQQKPDQAPKLLIYSGSTLQSGVPSRFSGSGSGTDFTFTISSLEAEDAATYYCQQHNKYPYTFGGGTKVEIK

SEQ ID NO: 55 - protein, 263 aa, Homo sapiens
MALPVTALLLPLALLLHAARPQVQLVQSGAEVKKPGASVKVSCKASGYTFTSYWMNWVRQAPGQGLEWMGRIDPYDSETHYNQKFKDRVTMTVDKSTSTAYMELSSLRSEDTAVYYCARGNWDDYWGQGTTVTVSSGGGGSGGGGSGGGGSGGGGSDVVMTQSPDSLAVSLGERATINCRASKSISKDLAWYQQKPGQPPKLLIYSGSTLQSGVPDRFSGSGSGTDFTLTISSLQAEDVAVYYCQQHNKYPYTFGGGTKVEIK

SEQ ID NO: 56 - protein, 263 aa, Homo sapiens
MALPVTALLLPLALLLHAARPDVQLTQSPSFLSASVGDRVTITCRASKSISKDLAWYQQKPGKAPKLLIYSGSTLQSGVPSRFSGSGSGTEFTLTISSLQPEDFATYYCQQHNKYPYTFGGGTKVEIKGGGGSGGGGSGGGGSGGGGSQVQLVQSGAEVKKPGASVKVSCKASGYTFTSYWMNWVRQAPGQGLEWMGRIDPYDSETHYNQKFKDRVTMTVDKSTSTAYMELSSLRSEDTAVYYCARGNWDDYWGQGTTVTVSS

SEQ ID NO: 57 - protein, 263 aa, Homo sapiens
MALPVTALLLPLALLLHAARPEVVLTQSPATLSLSPGERATLSCRASKSISKDLAWYQQKPGQAPRLLIYSGSTLQSGIPARFSGSGSGTDFTLTISSLEPEDFAVYYCQQHNKYPYTFGGGTKVEIKGGGGSGGGGSGGGGSGGGGSQVQLVQSGAEVKKPGASVKVSCKASGYTFTSYWMNWVRQAPGQGLEWMGRIDPYDSETHYNQKFKDRVTMTVDKSTSTAYMELSSLRSEDTAVYYCARGNWDDYWGQGTTVTVSS

SEQ ID NO: 58 - protein, 263 aa, Homo sapiens
MALPVTALLLPLALLLHAARPDVVMTQSPAFLSVTPGEKVTITCRASKSISKDLAWYQQKPDQAPKLLIYSGSTLQSGVPSRFSGSGSGTDFTFTISSLEAEDAATYYCQQHNKYPYTFGGGTKVEIKGGGGSGGGGSGGGGSGGGGSQVQLVQSGAEVKKPGASVKVSCKASGYTFTSYWMNWVRQAPGQGLEWMGRIDPYDSETHYNQKFKDRVTMTVDKSTSTAYMELSSLRSEDTAVYYCARGNWDDYWGQGTTVTVSS

SEQ ID NO: 59 - protein, 263 aa, Homo sapiens
MALPVTALLLPLALLLHAARPDVVMTQSPDSLAVSLGERATINCRASKSISKDLAWYQQKPGQPPKLLIYSGSTLQSGVPDRFSGSGSGTDFTLTISSLQAEDVAVYYCQQHNKYPYTFGGGTKVEIKGGGGSGGGGSGGGGSGGGGSQVQLVQSGAEVKKPGASVKVSCKASGYTFTSYWMNWVRQAPGQGLEWMGRIDPYDSETHYNQKFKDRVTMTVDKSTSTAYMELSSLRSEDTAVYYCARGNWDDYWGQGTTVTVSS

SEQ ID NO: 60 - protein, 263 aa, Homo sapiens
MALPVTALLLPLALLLHAARPQVQLVQSGSELKKPGASVKVSCKASGYTFTSYWMNWVRQAPGQGLEWMGRIDPYDSETHYNQKFKDRFVFSVDKSVSTAYLQISSLKAEDTAVYYCARGNWDDYWGQGTTVTVSSGGGGSGGGGSGGGGSGGGGSDVQLTQSPSFLSASVGDRVTITCRASKSISKDLAWYQQKPGKAPKLLIYSGSTLQSGVPSRFSGSGSGTEFTLTISSLQPEDFATYYCQQHNKYPYTFGGGTKVEIK

SEQ ID NO: 61 - protein, 263 aa, Homo sapiens
MALPVTALLLPLALLLHAARPQVQLVQSGSELKKPGASVKVSCKASGYTFTSYWMNWVRQAPGQGLEWMGRIDPYDSETHYNQKFKDRFVFSVDKSVSTAYLQISSLKAEDTAVYYCARGNWDDYWGQGTTVTVSSGGGGSGGGGSGGGGSGGGGSEVVLTQSPATLSLSPGERATLSCRASKSISKDLAWYQQKPGQAPRLLIYSGSTLQSGIPARFSGSGSGTDFTLTISSLEPEDFAVYYCQQHNKYPYTFGGGTKVEIK

SEQ ID NO: 62 - protein, 263 aa, Homo sapiens
MALPVTALLLPLALLLHAARPQVQLVQSGSELKKPGASVKVSCKASGYTFTSYWMNWVRQAPGQGLEWMGRIDPYDSETHYNQKFKDRFVFSVDKSVSTAYLQISSLKAEDTAVYYCARGNWDDYWGQGTTVTVSSGGGGSGGGGSGGGGSGGGGSDVVMTQSPAFLSVTPGEKVTITCRASKSISKDLAWYQQKPDQAPKLLIYSGSTLQSGVPSRFSGSGSGTDFTFTISSLEAEDAATYYCQQHNKYPYTFGGGTKVEIK

SEQ ID NO: 63 - protein, 263 aa, Homo sapiens
MALPVTALLLPLALLLHAARPQVQLVQSGSELKKPGASVKVSCKASGYTFTSYWMNWVRQAPGQGLEWMGRIDPYDSETHYNQKFKDRFVFSVDKSVSTAYLQISSLKAEDTAVYYCARGNWDDYWGQGTTVTVSSGGGGSGGGGSGGGGSGGGGSDVVMTQSPDSLAVSLGERATINCRASKSISKDLAWYQQKPGQPPKLLIYSGSTLQSGVPDRFSGSGSGTDFTLTISSLQAEDVAVYYCQQHNKYPYTFGGGTKVEIK

SEQ ID NO: 64 - protein, 263 aa, Homo sapiens
MALPVTALLLPLALLLHAARPDVQLTQSPSFLSASVGDRVTITCRASKSISKDLAWYQQKPGKAPKLLIYSGSTLQSGVPSRFSGSGSGTEFTLTISSLQPEDFATYYCQQHNKYPYTFGGGTKVEIKGGGGSGGGGSGGGGSGGGGSQVQLVQSGSELKKPGASVKVSCKASGYTFTSYWMNWVRQAPGQGLEWMGRIDPYDSETHYNQKFKDRFVFSVDKSVSTAYLQISSLKAEDTAVYYCARGNWDDYWGQGTTVTVSS

SEQ ID NO: 65 - protein, 263 aa, Homo sapiens
MALPVTALLLPLALLLHAARPEVVLTQSPATLSLSPGERATLSCRASKSISKDLAWYQQKPGQAPRLLIYSGSTLQSGIPARFSGSGSGTDFTLTISSLEPEDFAVYYCQQHNKYPYTFGGGTKVEIKGGGGSGGGGSGGGGSGGGGSQVQLVQSGSELKKPGASVKVSCKASGYTFTSYWMNWVRQAPGQGLEWMGRIDPYDSETHYNQKFKDRFVFSVDKSVSTAYLQISSLKAEDTAVYYCARGNWDDYWGQGTTVTVSS

SEQ ID NO: 66 - protein, 263 aa, Homo sapiens
MALPVTALLLPLALLLHAARPDVVMTQSPAFLSVTPGEKVTITCRASKSISKDLAWYQQKPDQAPKLLIYSGSTLQSGVPSRFSGSGSGTDFTFTISSLEAEDAATYYCQQHNKYPYTFGGGTKVEIKGGGGSGGGGSGGGGSGGGGSQVQLVQSGSELKKPGASVKVSCKASGYTFTSYWMNWVRQAPGQGLEWMGRIDPYDSETHYNQKFKDRFVFSVDKSVSTAYLQISSLKAEDTAVYYCARGNWDDYWGQGTTVTVSS

SEQ ID NO: 67 - protein, 263 aa, Homo sapiens
MALPVTALLLPLALLLHAARPDVVMTQSPDSLAVSLGERATINCRASKSISKDLAWYQQKPGQPPKLLIYSGSTLQSGVPDRFSGSGSGTDFTLTISSLQAEDVAVYYCQQHNKYPYTFGGGTKVEIKGGGGSGGGGSGGGGSGGGGSQVQLVQSGSELKKPGASVKVSCKASGYTFTSYWMNWVRQAPGQGLEWMGRIDPYDSETHYNQKFKDRFVFSVDKSVSTAYLQISSLKAEDTAVYYCARGNWDDYWGQGTTVTVSS

SEQ ID NO: 68 - protein, 263 aa, Homo sapiens
MALPVTALLLPLALLLHAARPEVQLVQSGAEVKKPGESLRISCKGSGYTFTSYWMNWVRQMPGKGLEWMGRIDPYDSETHYNQKFKDHVTISVDKSISTAYLQWSSLKASDTAMYYCARGNWDDYWGQGTTVTVSSGGGGSGGGGSGGGGSGGGGSDVQTQSPSFLSASVGDRVITCRASKSISKDLAWLTYQQKPGKAPKLLIYSGSTLQSGVPSRFSGSGSGTEFTLTISSLQPEDFATYYCQQHNKYPYTFGGGTKVEIK

SEQ ID NO: 69 - protein, 263 aa, Homo sapiens
MALPVTALLLPLALLLHAARPEVQLVQSGAEVKKPGESLRISCKGSGYTFTSYWMNWVRQMPGKGLEWMGRIDPYDSETHYNQKFKDHISTAYLQWVSTISVDSLKASKSDTAMYYCARGNWDDYWGQGTTVTVSSGGGGSGGGGSGGGGSGGGGSEVVLTQSPATLSLSPGERATLSCRASKSISKDLAWYQQKPGQAPRLLIYSGSTLQSGIPARFSGSGSGTDFTLTISSLEPEDFAVYYCQQHNKYPYTFGGGTKVEIK

SEQ ID NO: 70 - protein, 263 aa, Homo sapiens
MALPVTALLLPLALLLHAARPEVQLVQSGAEVKKPGESLRISCKGSGYTFTSYWMNWVRQMPGKGLEWMGRIDPYDSETHYNQKFKDHVTISVDKSISTAYLQWSSLKASDTAMYYCARGNWDDYWGQGTTVTVSSGGGGSGGGGSGGGGSGGGGSDVVMTQSPAFLSVTPGEKVTITCRASKSISKDLAWYQQKPDQAPKLLIYSGSTLQSGVPSRFSGSGSGTDFTFTISSLEAEDAATYYCQQHNKYPYTFGGGTKVEIK

SEQ ID NO: 71 - protein, 263 aa, Homo sapiens
MALPVTALLLPLALLLHAARPEVQLVQSGAEVKKPGESLRISCKGSGYTFTSYWMNWVRQMPGKGLEWMGRIDPYDSETHYNQKFKDHVTISVDKSISTAYLQWSSLKASDTAMYYCARGNWDDYWGQGTTVTVSSGGGGSGGGGSGGGGSGGGGSDVVMTQSPDSLAVSLGERATINCRASKSISKDLAWYQQKPGQPPKLLIYSGSTLQSGVPDRFSGSGSGTDFTLTISSLQAEDVAVYYCQQHNKYPYTFGGGTKVEIK

SEQ ID NO: 72 - protein, 263 aa, Homo sapiens
MALPVTALLLPLALLLHAARPDVQLTQSPSFLSASVGDRVTITCRASKSISKDLAWYQQKPGKAPKLLIYSGSTLQSGVPSRFSGSGSGTEFTLTISSLQPEDFATYYCQQHNKYPYTFGGGTKVEIKGGGGSGGGGSGGGGSGGGGSEVQLVQSGAEVKKPGESLRISCKGSGYTFTSYWMNWVRQMPGKGLEWMGRIDPYDSETHYNQKFKDHVTISVDKSISTAYLQWSSLKASDTAMYYCARGNWDDYWGQGTTVTVSS

SEQ ID NO: 73 - protein, 263 aa, Homo sapiens
MALPVTALLLPLALLLHAARPEVVLTQSPATLSLSPGERATLSCRASKSISKDLAWYQQKPGQAPRLLIYSGSTLQSGIPARFSGSGSGTDFTLTISSLEPEDFAVYYCQQHNKYPYTFGGGTKVEIKGGGGSGGGGSGGGGSGGGGSEVQLVQSGAEVKKPGESLRISCKGSGYTFTSYWMNWVRQMPGKGLEWMGRIDPYDSETHYNQKFKDHVTISVDKSISTAYLQWSSLKASDTAMYYCARGNWDDYWGQGTTVTVSS

SEQ ID NO: 74 - protein, 263 aa, Homo sapiens
MALPVTALLLPLALLLHAARPDVVMTQSPAFLSVTPGEKVTITCRASKSISKDLAWYQQKPDQAPKLLIYSGSTLQSGVPSRFSGSGSGTDFTFTISSLEAEDAATYYCQQHNKYPYTFGGGTKVEIKGGGGSGGGGSGGGGSGGGGSEVQLVQSGAEVKKPGESLRISCKGSGYTFTSYWMNWVRQMPGKGLEWMGRIDPYDSETHYNQKFKDHVTISVDKSISTAYLQWSSLKASDTAMYYCARGNWDDYWGQGTTVTVSS

SEQ ID NO: 75 - protein, 263 aa, Homo sapiens
MALPVTALLLPLALLLHAARPDVVMTQSPDSLAVSLGERATINCRASKSISKDLAWYQQKPGQPPKLLIYSGSTLQSGVPDRFSGSGSGTDFTLTISSLQAEDVAVYYCQQHNKYPYTFGGGTKVEIKGGGGSGGGGSGGGGSGGGGSEVQLVQSGAEVKKPGESLRISCKGSGYTFTSYWMNWVRQMPGKGLEWMGRIDPYDSETHYNQKFKDHVTISVDKSISTAYLQWSSLKASDTAMYYCARGNWDDYWGQGTTVTVSS

SEQ ID NO: 76 - protein, 263 aa, Homo sapiens
MALPVTALLLPLALLLHAARPEVQLVESGGGLVQPGGSLRLSCAASGYTFTSYWMNWVRQAPGKGLVWVSRIDPYDSETHYNQKFKDRFTISVDKAKSTAYLQMNSLRAEDTAVYYCARGNWDDYWGQGTTVTVSSGGGGSGGGGSGGGGSGGGGSDVQLTQSPSFLSASVGDRVTITCRASKSISKDLAWYQQKPGKAPKLLIYSGSTLQSGVPSRFSGSGSGTEFTLTISSLQPEDFATYYCQQHNKYPYTFGGGTKVEIK

SEQ ID NO: 77 - protein, 263 aa, Homo sapiens
MALPVTALLLPLALLLHAARPEVQLVESGGGLVQPGGSLRLSCAASGYTFTSYWMNWVRQAPGKGLVWVSRIDPYDSETHYNQKFKDRFTISVDKAKSTAYLQMNSLRAEDTAVYYCARGNWDDYWGQGTTVTVSSGGGGSGGGGSGGGGSGGGGSEVVLTQSPATLSLSPGERATLSCRASKSISKDLAWYQQKPGQAPRLLIYSGSTLQSGIPARFSGSGSGTDFTLTISSLEPEDFAVYYCQQHNKYPYTFGGGTKVEIK

SEQ ID NO: 78 - protein, 263 aa, Homo sapiens
MALPVTALLLPLALLLHAARPEVQLVESGGGLVQPGGSLRLSCAASGYTFTSYWMNWVRQAPGKGLVWVSRIDPYDSETHYNQKFKDRFTISVDKAKSTAYLQMNSLRAEDTAVYYCARGNWDDYWGQGTTVTVSSGGGGSGGGGSGGGGSGGGGSDVVMTQSPAFLSVTPGEKVTITCRASKSISKDLAWYQQKPDQAPKLLIYSGSTLQSGVPSRFSGSGSGTDFTFTISSLEAEDAATYYCQQHNKYPYTFGGGTKVEIK

SEQ ID NO: 79 - protein, 263 aa, Homo sapiens
MALPVTALLLPLALLLHAARPEVQLVESGGGLVQPGGSLRLSCAASGYTFTSYWMNWVRQAPGKGLVWVSRIDPYDSETHYNQKFKDRFTISVDKAKSTAYLQMNSLRAEDTAVYYCARGNWDDYWGQGTTVTVSSGGGGSGGGGSGGGGSGGGGSDVVMTQSPDSLAVSLGERATINCRASKSISKDLAWYQQKPGQPPKLLIYSGSTLQSGVPDRFSGSGSGTDFTLTISSLQAEDVAVYYCQQHNKYPYTFGGGTKVEIK

SEQ ID NO: 80 - protein, 263 aa, Homo sapiens
MALPVTALLLPLALLLHAARPDVQLTQSPSFLSASVGDRVTITCRASKSISKDLAWYQQKPGKAPKLLIYSGSTLQSGVPSRFSGSGSGTEFTLTISSLQPEDFATYYCQQHNKYPYTFGGGTKVEIKGGGGSGGGGSGGGGSGGGGSEVQLVESGGGLVQPGGSLRLSCAASGYTFTSYWMNWVRQAPGKGLVWVSRIDPYDSETHYNQKFKDRFTISVDKAKSTAYLQMNSLRAEDTAVYYCARGNWDDYWGQGTTVTVSS

SEQ ID NO: 81 - protein, 263 aa, Homo sapiens
MALPVTALLLPLALLLHAARPEVVLTQSPATLSLSPGERATLSCRASKSISKDLAWYQQKPGQAPRLLIYSGSTLQSGIPARFSGSGSGTDFTLTISSLEPEDFAVYYCQQHNKYPYTFGGGTKVEIKGGGGSGGGGSGGGGSGGGGSEVQLVESGGGLVQPGGSLRLSCAASGYTFTSYWMNWVRQAPGKGLVWVSRIDPYDSETHYNQKFKDRFTISVDKAKSTAYLQMNSLRAEDTAVYYCARGNWDDYWGQGTTVTVSS

SEQ ID NO: 82 - protein, 263 aa, Homo sapiens
MALPVTALLLPLALLLHAARPDVVMTQSPAFLSVTPGEKVTITCRASKSISKDLAWYQQKPDQAPKLLIYSGSTLQSGVPSRFSGSGSGTDFTFTISSLEAEDAATYYCQQHNKYPYTFGGGTKVEIKGGGGSGGGGSGGGGSGGGGSEVQLVESGGGLVQPGGSLRLSCAASGYTFTSYWMNWVRQAPGKGLVWVSRIDPYDSETHYNQKFKDRFTISVDKAKSTAYLQMNSLRAEDTAVYYCARGNWDDYWGQGTTVTVSS

SEQ ID NO: 83 - protein, 263 aa, Homo sapiens
MALPVTALLLPLALLLHAARPDVVMTQSPDSLAVSLGERATINCRASKSISKDLAWYQQKPGQPPKLLIYSGSTLQSGVPDRFSGSGSGTDFTLTISSLQAEDVAVYYCQQHNKYPYTFGGGTKVEIKGGGGSGGGGSGGGGSGGGGSEVQLVESGGGLVQPGGSLRLSCAASGYTFTSYWMNWVRQAPGKGLVWVSRIDPYDSETHYNQKFKDRFTISVDKAKSTAYLQMNSLRAEDTAVYYCARGNWDDYWGQGTTVTVSS

SEQ ID NO: 84 - protein, 113 aa, Homo sapiens
MADYKDIVMTQSPSSVSASVGDRVTITCRASQNVDSAVAWYQQKPGKAPKALIYSASYRYSGVPSRFSGRGSGTDFTLTISSLQPEDFATYYCQQYYSTPWTFGQGTKVEIKR

SEQ ID NO: 85 - protein, 113 aa, Homo sapiens
MADYKDIQMTQSPSSVSASVGDRVTITCRASQNVDSAVAWYQQKPGKAPKALIYSASYRYSGVPSRFSGRGSGTDFTLTISSLQPEDFATYYCQQYYSTPWTFGQGTKVEIKR

SEQ ID NO: 86 - protein, 113 aa, Homo sapiens
MADYKDIQMTQSPSSVSASVGDRVTITCRASQNVDSAVAWYQQKPGKAPKALIYSASYRYSGVPSRFSGSGSGTDFTLTISSLQPEDFATYYCQQYYSTPWTFGQGTKVEIKR

SEQ ID NO: 87 - protein, 113 aa, Homo sapiens
MADYKDIQMTQSPSSVSASVGDRVTITCRASQNVDSAVAWYQQKPGKAPKLLIYSASYRYSGVPSRFSGSGSGTDFTLTISSLQPEDFATYYCQQYYSTPWTFGQGTKVEIKR

SEQ ID NO: 88 - protein, 113 aa, Homo sapiens
MADYKDIQMTQSPSSVSASVGDRVTITCRASQNVDSAVAWYQQKPGKAPKLLIYSASYRQSGVPSRFSGSGSGTDFTLTISSLQPEDFATYYCQQYYSTPWTFGQGTKVEIKR

SEQ ID NO: 89 - protein, 113 aa, Homo sapiens
MADYKDIQMTQSPSSVSASVGDRVTITCRASQNVDSAVAWYQQKPGKAPKLLIYSASYGQSGVPSRFSGSGSGTDFTLTISSLQPEDFATYYCQQYYSTPWTFGQGTKVEIKR

SEQ ID NO: 90 - protein, 127 aa, Homo sapiens
EVKLVESGGGLVQPGRSLRLSCTASGFTFTDYYMSWVRQAPGKGLEWVGLIRSKADGYTTEYSASVKGRFTISRDDSKSILYLQMNSLKTEDTAVYYCARDAAYYSYYSPEGAMDYWGQGTLVTVSS

SEQ ID NO: 91 - protein, 127 aa, Homo sapiens
EVQLVESGGGLVQPGRSLRLSCTASGFTFTDYYMSWVRQAPGKGLEWVGLIRSKADGYTTEYSASVKGRFTISRDDSKSILYLQMNSLKTEDTAVYYCARDAAYYSYYSPEGAMDYWGQGTLVTVSS

SEQ ID NO: 92 - protein, 127 aa, Homo sapiens
EVQLVESGGGLVQPGRSLRLSCTASGFTFTDYYMSWVRQAPGKGLEWVGLIRSKADGYTTEYSASVKGRFTISRDDSKSIAYLQMNSLKTEDTAVYYCARDAAYYSYYSPEGAMDYWGQGTLVTVSS

SEQ ID NO: 93 - protein, 127 aa, Homo sapiens
EVQLVESGGGLVQPGRSLRLSCTASGFTFTDYYMSWVRQAPGKGLEWVGFIRSKADGYTTEYSASVKGRFTISRDDSKSIAYLQMNSLKTEDTAVYYCARDAAYYSYYSPEGAMDYWGQGTLVTVSS

SEQ ID NO: 94 - protein, 127 aa, Homo sapiens
EVQLVESGGGLVQPGRSLRLSCTASGFTFTDYYMSWVRQAPGKGLEWVGFIRSKADGYTTEYAASVKGRFTISRDDSKSIAYLQMNSLKTEDTAVYYCARDAAYYSYYSPEGAMDYWGQGTLVTVSS

SEQ ID NO: 95 - protein, 127 aa, Homo sapiens
EVQLVESGGGLVQPGRSLRLSCTASGFTFTDYYMSWVRQAPGKGLEWVGLIRSKADGYTTEYAASVKGRFTISRDDSKSIAYLQMNSLKTEDTAVYYCARDAAYYSYYSPEGAMDYWGQGTLVTVSS

SEQ ID NO: 96 - protein, 127 aa, Homo sapiens
EVQLVESGGGLVQPGRSLRLSCTASGFTFTDYYMSWVRQAPGKGLEWVGFIRSKADGYTTEYAASVKGRFTISRDDSKSIAYLQMNSLKTEDTAVYYCTRDAAYYSYYSPEGAMDYWGQGTLVTVSS

SEQ ID NO: 97 - protein, 8 aa, Homo sapiens
GFTFTDYY

SEQ ID NO: 98 - protein, 9 aa, Homo sapiens
RSKADGYTT

SEQ ID NO: 99 - protein, 16 aa, Homo sapiens
ARDAAYYSYYSPEGAM

SEQ ID NO: 100 - protein, 6 aa, Homo sapiens
QNVDSA

SEQ ID NO: 101 - skipped

SEQ ID NO: 102 - protein, 9 aa, Homo sapiens
QQYYSTPWT

SEQ ID NO: 103 - protein, 271 aa, Homo sapiens
MALPVTALLLPLALLLHAARPQVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARLGGSLPDYGMDVWGQGTMVTVSSASGGGGSGGGGSGGGGSEIVLTQPASISSPLSLPVTPCRSSQSLLHGESNGYNYLDWYLQKPGQSPQLLIYLGSNRASGVPDRFSGSGSGTDFTLKISRVEAEDVGVYYCMQALQTLITFGQGTKVDIK

SEQ ID NO: 104 - protein, 272 aa, Homo sapiens
MALPVTALLLPLALLLHAARPQVQLVQSGAEVKKPGASVRVSCKASGYIFTNYYVHWVRQAPGQGLEWMGIISPSGGSPTYAQRLQGRVTMTRDLSTSTVYMELSSLTSEDTAVYFCARESRLRGNRLGLQSSIFDHWGQGTLVTVSSASGGGGSGGGGSGGGGSDIRMTQSPPSLSASVGDRVTIPCQASQDINNHLNWYQQKPGKAPQLLIYDTSNLEIGVPSRFSGSGSGTDFTLTISSLQPEDIATYYCQQYENLPLTFGGGTKVEIK

SEQ ID NO: 105 - protein, 270 aa, Homo sapiens
MALPVTALLLPLALLLHAARPQVQLVQSGGGLVQPGGSLRLSCAASGFTFSSYAMSWVRQAPGKGLEWVSAISGSGGSTYYADSVKGRFTISRDNSKNTLYLQMNSLRAEDTAVYYCAKEDTIRGPNYYYYGMDVWGQGTTVTVSSASGGGGSGGGGSGGGGSETTLTQSPSSVSASVGDRVSITCRASQDIDTWLAWYQLKPGKAPKLLMYAASNLQGGVPSRFSGSGSGTDFILTISSLQPEDFATYYCQQASIFPPTFGGGTKVDIK

SEQ ID NO: 106 - protein, 271 aa, Homo sapiens
MALPVTALLLPLALLLHAARPQVQLVQSGAEVKKPGESLKISCKGSGYSFTSYWIGWVRQMPGKGLEWMGIIYPGDSDTRYSPSFQGQVTISADKSITTAYLQWSSLRASDSAMYYCARGGYSDYDYYFDFWGQGTLVTVSSASGGGGSGGGGSGGGGSEIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPFTFGGGTKVEIK

SEQ ID NO: 107 - protein, 261 aa, Homo sapiens
MALPVTALLLPLALLLHAARPQVQLVQSGGDLAQPGRSLRLSCAASGFTFDDYAMHWVRQAPGKGLEWVAVIWPDGGQKYYGDSVKGRFTVSRDNPKNTLYLQMNSLRAEDTAIYYCVRHFNAWDYWGQGTLVTVSSASGGGGSGGGGSGGGGSDIQLTQSPSSLSAYVGGRVTITCQASQGISQFLNWFQQKPGKAPKLLISDASNLEPGVPSRFSGSGSGTDFTFTITNLQPEDIATYYCQQYDDLPLTFGGGTKVEIK

SEQ ID NO: 108 - protein, 269 aa, Homo sapiens
MALPVTALLLPLALLLHAARPQVQLVQSGGGVVQPGKSLRLSCAASGFTFSIFAMHWVRQAPGKGLEWVATISYDGSNAFYADSVEGRFTISRDNSKDSLYLQMDSLRPEDTAVYYCVKAGDGGYDVFDSWGQGTLVTVSSASGGGGSGGGGSGGGGSEIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPTFGPGTKVDIK

SEQ ID NO: 109 - protein, 266 aa, Homo sapiens
MALPVTALLLPLALLLHAARPEVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCAKETDYYGSGTFDYWGQGTLVTVSSASGGGGSGGGGSGGGGSDIQMTQSPSSLSASVGDRVTISCRASQGIGIYLAWYQQRSGKPPQLLIHGASTLQSGVPSRFSGSGSGTDFTLTISSLQPEDFASYWCQQSNNFPPTFGQGTKVEIK

SEQ ID NO: 110 - protein, 266 aa, Homo sapiens
MALPVTALLLPLALLLHAARPQVQLVQSGAEVKKPGASVRVSCKASGYMFTDFFIHWVRQAPGQGLEWMGWINPNSGVTKYAQKFQGRVTMTRNTSISTAYMELSSLRSEDTAVYYCATWYSSGWYGIANIWGQGTMVTVSSASGGGGSGGGGSGGGGSDIQLTQSPSSLSASVGDRVTITCQASHDISNYLHWYQQKPGKAPKLLIYDASNLETGVPSRFTGSGSGTDFTLTIRSLQPEDVAAYYCQQSDDLPHTFGQGTKVDIK

SEQ ID NO: 111 - protein, 265 aa, Homo sapiens
MALPVTALLLPLALLLHAARPQVQLVQSGAEVKKPGESLKISCKGSGYSFTNYWIGWVRQMPGKGLEWMGIIYPGDSDTRYSPSFQGQVTISADKSISTAYLQWSSLKASDTAMYYCARHGPSSWGEFDYWGQGTLVTVSSASGGGGSGGGGSGGGGSDIRLTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVDIK

SEQ ID NO: 112 - protein, 121 aa, Homo sapiens
QVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARLGGSLPDYGMDVWGQGTMVTVSS

SEQ ID NO: 113 - protein, 127 aa, Homo sapiens
QVQLVQSGAEVKKPGASVRVSCKASGYIFTNYYVHWVRQAPGQGLEWMGIISPSGGSPTYAQRLQGRVTMTRDLSTSTVYMELSSLTSEDTAVYFCARESRLRGNRLGLQSSIFDHWGQGTLVTVSS

SEQ ID NO: 114 - protein, 125 aa, Homo sapiens
QVQLVQSGGGLVQPGGSLRLSCAASGFTFSSYAMSWVRQAPGKGLEWVSAISGSGGSTYYADSVKGRFTISRDNSKNTLYLQMNSLRAEDTAVYYCAKEDTIRGPNYYYYGMDVWGQGTTVTVSS

SEQ ID NO: 115 - protein, 121 aa, Homo sapiens
QVQLVQSGAEVKKPGESLKISCKGSGYSFTSYWIGWVRQMPGKGLEWMGIIYPGDSDTRYSPSFQGQVTISADKSITTAYLQWSSLRASDSAMYYCARGGYSDYDYYFDFWGQGTLVTVSS

SEQ ID NO: 116 - protein, 116 aa, Homo sapiens
QVQLVQSGGDLAQPGRSLRLSCAASGFTFDDYAMHWVRQAPGKGLEWVAVIWPDGGQKYYGDSVKGRFTVSRDNPKNTLYLQMNSLRAEDTAIYYCVRHFNAWDYWGQGTLVTVSS

SEQ ID NO: 117 - protein, 120 aa, Homo sapiens
QVQLVQSGGGVVQPGKSLRLSCAASGFTFSIFAMHWVRQAPGKGLEWVATISYDGSNAFYADSVEGRFTISRDNSKDSLYLQMDSLRPEDTAVYYCVKAGDGGYDVFDSWGQGTLVTVSS

SEQ ID NO: 118 - protein, 121 aa, Homo sapiens
EVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCAKETDYYGSGTFDYWGQGTLVTVSS

SEQ ID NO: 119 - protein, 121 aa, Homo sapiens
QVQLVQSGAEVKKPGASVRVSCKASGYMFTDFFIHWVRQAPGQGLEWMGWINPNSGVTKYAQKFQGRVTMTRNTSISTAYMELSSLRSEDTAVYYCATWYSSGWYGIANIWGQGTMVTVSS

SEQ ID NO: 120 - protein, 120 aa, Homo sapiens
QVQLVQSGAEVKKPGESLKISCKGSGYSFTNYWIGWVRQMPGKGLEWMGIIYPGDSDTRYSPSFQGQVTISADKSISTAYLQWSSLKASDTAMYYCARHGPSSWGEFDYWGQGTLVTVSS

SEQ ID NO: 121 - protein, 112 aa, Homo sapiens
EIVLTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTLITFGQGTKVDIK

SEQ ID NO: 122 - protein, 107 aa, Homo sapiens
DIRMTQSPPSLSASVGDRVTIPCQASQDINNHLNWYQQKPGKAPQLLIYDTSNLEIGVPSRFSGSGSGTDFTLTISSLQPEDIATYYCQQYENLPLTFGGGTKVEIK

SEQ ID NO: 123 - protein, 107 aa, Homo sapiens
ETTLTQSPSSVSASVGDRVSITCRASQDIDTWLAWYQLKPGKAPKLLMYAASNLQGGVPSRFSGSGSGTDFILTISSLQPEDFATYYCQQASIFPPTFGGGTKVDIK

SEQ ID NO: 124 - protein, 112 aa, Homo sapiens
EIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPFTFGGGTKVEIK

SEQ ID NO: 125 - protein, 107 aa, Homo sapiens
DIQLTQSPSSLSAYVGGRVTITCQASQGISQFLNWFQQKPGKAPKLLISDASNLEPGVPSRFSGSGSGTDFTFTITNLQPEDIATYYCQQYDDLPLTFGGGTKVEIK

SEQ ID NO: 126 - protein, 111 aa, Homo sapiens
EIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPTFGPGTKVDIK

SEQ ID NO: 127 - protein, 107 aa, Homo sapiens
DIQMTQSPSSLSASVGDRVTISCRASQGIGIYLAWYQQRSGKPPQLLIHGASTLQSGVPSRFSGSGSGTDFTLTISSLQPEDFASYWCQQSNNFPPTFGQGTKVEIK

SEQ ID NO: 128 - protein, 107 aa, Homo sapiens
DIQLTQSPSSLSASVGDRVTITCQASHDISNYLHWYQQKPGKAPKLLIYDASNLETGVPSRFTGSGSGTDFTLTIRSLQPEDVAAYYCQQSDDLPHTFGQGTKVDIK

SEQ ID NO: 129 - protein, 107 aa, Homo sapiens
DIRLTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVDIK

SEQ ID NO: 130 - protein, 250 aa, Homo sapiens
QVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARLGGSLPDYGMDVWGQGTMVTVSSASGGGGSGGGGSGGGGSEIVLTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTLITFGQGTKVDIK

SEQ ID NO: 131 - protein, 251 aa, Homo sapiens
QVQLVQSGAEVKKPGASVRVSCKASGYIFTNYYVHWVRQAPGQGLEWMGIISPSGGSPTYAQRLQGRVTMTRDLSTSTVYMELSSLTSEDTAVYFCARESRLRGNRLGLQSSIFDHWGQGTLVTVSSASGGGGSGGGGSGGGGSDIRMTQSPPSLSASVGDRVTIPCQASQDINNHLNWYQQKPGKAPQLLIYDTSNLEIGVPSRFSGSGSGTDFTLTISSLQPEDIATYYCQQYENLPLTFGGGTKVEIK

SEQ ID NO: 132 - protein, 250 aa, Homo sapiens
QVQLVQSGAEVKKPGESLKISCKGSGYSFTSYWIGWVRQMPGKGLEWMGIIYPGDSDTRYSPSFQGQVTISADKSITTAYLQWSSLRASDSAMYYCARGGYSDYDYYFDFWGQGTLVTVSSASGGGGSGGGGSGGGGSEIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPFTFGGGTKVEIK

SEQ ID NO: 133 - protein, 240 aa, Homo sapiens
QVQLVQSGGDLAQPGRSLRLSCAASGFTFDDYAMHWVRQAPGKGLEWVAVIWPDGGQKYYGDSVKGRFTVSRDNPKNTLYLQMNSLRAEDTAIYYCVRHFNAWDYWGQGTLVTVSSASGGGGSGGGGSGGGGSDIQLTQSPSSLSAYVGGRVTITCQASQGISQFLNWFQQKPGKAPKLLISDASNLEPGVPSRFSGSGSGTDFTFTITNLQPEDIATYYCQQYDDLPLTFGGGTKVEIK

SEQ ID NO: 134 - protein, 248 aa, Homo sapiens
QVQLVQSGGGVVQPGKSLRLSCAASGFTFSIFAMHWVRQAPGKGLEWVATISYDGSNAFYADSVEGRFTISRDNSKDSLYLQMDSLRPEDTAVYYCVKAGDGGYDVFDSWGQGTLVTVSSASGGGGSGGGGSGGGGSEIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPTFGPGTKVDIK

SEQ ID NO: 135 - protein, 245 aa, Homo sapiens
EVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCAKETDYYGSGTFDYWGQGTLVTVSSASGGGGSGGGGSGGGGSDIQMTQSPSSLSASVGDRVTISCRASQGIGIYLAWYQQRSGKPPQLLIHGASTLQSGVPSRFSGSGSGTDFTLTISSLQPEDFASYWCQQSNNFPPTFGQGTKVEIK

SEQ ID NO: 136 - protein, 244 aa, Homo sapiens
QVQLVQSGAEVKKPGESLKISCKGSGYSFTNYWIGWVRQMPGKGLEWMGIIYPGDSDTRYSPSFQGQVTISADKSISTAYLQWSSLKASDTAMYYCARHGPSSWGEFDYWGQGTLVTVSSASGGGGSGGGGSGGGGSDIRLTQSPSSLSASVGDRVTITCRASQPGKASGVPSISSYLNWYQPKLLIYAASSSRFSGSGSGTQKLQDFTLTISSLQPEDFATYYCQQSYSTPLTFGGGTKVDIK

(19) United States
(12) Patent Application Publication
(10) Pub. No.: US 2022/0160771 A1
(43) Pub. Date: May 26, 2022

(54) FLT3-SPECIFIC CHIMERIC ANTIGEN RECEPTORS AND METHODS OF USING THE SAME

(71) Applicants: Iulia DIACONU, Cambridge, MA (US); Cherylene PLEWA, Cambridge, MA (US); Sandra MILASTA, Cambridge, MA (US); ELEVATEBIO MANAGEMENT, INC., Cambridge, MA (US)

(72) Inventors: Iulia DIACONU, Cambridge, MA (US); Cherylene PLEWA, Cambridge, MA (US); Patrick BAEUERLE, Cambridge, MA (US); Sandra MILASTA, Cambridge, MA (US); Jed WILTZIUS, Cambridge, MA (US)

(21) Appl. No.: 17/602,652

(22) PCT Filed: Apr. 10, 2020
(86) PCT No.: PCT/US2020/027796

§ 371 Date: Oct. 8, 2021

Related U.S. Application Data
(60) Provisional application No. 62/920,038, filed on Apr. 10, 2019.

(51) Int. Cl. A61K 35/17; C07K 16/28; C07K 16/40; C12N 15/86; C07K 14/725

(52) U.S. Cl. CPC A61K 35/17; C07K 16/2863; C07K 16/2896; C07K 16/40; C12N 15/86; C07K 2319/50; C07K 2317/31; C07K 2317/622; C07K 2317/73; C07K 2319/33; C07K 2319/03; C07K 14/7051

(57) ABSTRACT

Some embodiments of the disclosure provides a chimeric antigen receptor (CAR) comprising an antigen binding domain specific for FLT3, an antigen binding domain specific for a second antigen, a transmembrane domain, and an intracellular T cell signaling domain. Some embodiments of the disclosure provides bicistronic CARs. Nucleic acids, recombinant expression vectors, host cells, populations of cells, antibodies, or antigen binding portions thereof, and pharmaceutical compositions relating to the CARs are disclosed. Methods of detecting the presence of a proliferative disorder, e.g., cancer, in a mammal and methods of treating or preventing a proliferative disorder, e.g., cancer, in a mammal are also disclosed.

### RELATED APPLICATIONS

This application claims the benefit of provisional application U.S. Ser. No. 62/920,038, filed Apr. 10, 2019, the contents of which are herein incorporated by reference in their entirety.

### INCORPORATION OF SEQUENCE LISTING

The contents of the text file named “SWBK-002/01WO_SeqList.txt,” which was created on Apr. 10, 2020 and is 213 KB in size, are hereby incorporated by reference in their entirety.

### FIELD OF THE DISCLOSURE

The present disclosure is directed to compositions and methods for treatment of cancer, including. Acute Lymphoblastic Leukemia (ALL) and Acute Myeloid Leukemia (AML) by a combination therapy of a FLT-3 chimeric antigen receptor (CAR) either bi-cistronic for a second antigen or provided in combination with a CAR specific for a second antigen.

### BACKGROUND

Acute Lymphoblastic Leukemia (ALL) and Acute Myeloid Leukemia (AML) remain a common cause of death from cancer in children due to relapse of disease that no longer responds to cytotoxic chemotherapy, or due to refractories to upfront treatment. Furthermore, long-term therapy-induced morbidity remains a major issue, particularly those patients deemed to be high-risk for relapse and thus treated with more intense regimens under current risk-adapted protocols. In adults, ALL and AML occur less commonly than in children, but the prognosis for adult ALL or AML is worse than in children undergoing standard cytotoxic chemotherapy.

Thus, a long-felt and unmet need remains for an efficacious treatment for ALL and AML in patients of all ages that does not cause cytotoxicity or induce a refractory response. The disclosure provides compositions and methods address this need in the art.

### SUMMARY

The disclosure provides compositions and methods for the treatment of cancer, including Acute Lymphoblastic Leukemia (ALL) and Acute Myeloid Leukemia (AML).

The disclosure provides a bispecific chimeric antigen receptor (CAR), comprising an antigen binding domain specific for FLT3, an antigen binding domain specific for a second antigen, a transmembrane domain, and an intracellular T cell signaling domain. The CAR may further comprise a 4-1BB intracellular domain, a linker, a spacer, or a combination thereof.

Bispecific CAR-Ts of the disclosure, when administered to a subject, may reduce the risk of antigen-negative escape/resistance that could arise during treatment with a monovalent CAR-T.

The disclosure provides a CAR comprising (a) a cleavable domain; (b) a first CAR comprising a first antigen binding domain, a first transmembrane domain, and a first intracellular T cell signaling domain; and (c) a second CAR comprising a second antigen binding domain, a second transmembrane domain, and a second intracellular T cell signaling domain; wherein the first and second CARs are linked through the cleavable domain, wherein when the first CAR is cleaved from the construct, the first antigen binding domain has antigenic specificity for FLT3.

The disclosure provides population of cells comprising a first immune cell comprising a first nucleic acid molecule encoding a first CAR comprising an antigen binding domain with specificity for FLT3 and an intracellular T cell signaling domain, and a second immune cell comprising a second nucleic acid molecule encoding a second a second CAR comprising an antigen binding domain with specificity for a second antigen and an intracellular T cell signaling domain.

The disclosure provides related nucleic acids, recombinant expression vectors, host cells, populations of cells, antibodies, or antigen binding portions thereof, and pharmaceutical compositions relating to the CARs of the disclosure.

The disclosure provides methods of detecting the presence of a proliferative disorder, e.g., cancer, and methods of treating or preventing a proliferative disorder, e.g., cancer, in a mammal.

The disclosure provides a nucleic acid molecule encoding a chimeric antigen receptor (CAR) comprising: an antigen binding domain specific for FLT3 or a sequence encoding the antigen binding domain specific for FLT3, an antigen binding domain specific for a second antigen or a sequence encoding the antigen binding domain specific for a second antigen, a transmembrane domain or a sequence encoding the transmembrane domain, and an intracellular T cell signaling domain or a sequence encoding the intracellular T cell signaling domain. In some embodiments, the second antigen is selected from the group consisting of CD antigen 19 (CD19), CD antigen 22 (CD22), CD antigen 33 (CD33), CD antigen 123 (CD123), Lewis-Y, CD antigen 44, isoform 6 (CD44v6), C-type lectin-like molecule 1 (CLL-1), folate receptor-beta (FOLR2), CD antigen 13 (CD13), CD antigen 15 (CD15), CD antigen 30 (CD30), CD antigen 45 (CD45), CD antigen 47 (CD47), Angiopoietin-2 (Ang-2) CD antigen 133 (CD133), Wilms tumor protein (WT1), Vascular endothelial growth factor A (VEGF-A), U5 small nuclear ribonucleoprotein 200 kDa helicase (U5 snRNP200), Adhesion G protein-coupled receptor E2 (ADGRE2), CD antigen 38 (CD38), CD antigen 157 (CD157) Leukocyte immunoglobulin-like receptor subfamily B member 2 (LILRB2), C-C chemokine receptor type 1 (CCR-1), Proto-oncogene C-KIT, and CD antigen 43 (CD43).

In some embodiments of the nucleic acid molecules encoding a CAR of the disclosure, the CAR comprises an antigen binding domain specific for FLT3 and an antigen binding domain specific for CD19.

In some embodiments of the nucleic acid molecules encoding a CAR of the disclosure, the CAR comprises an antigen binding domain specific for FLT3 and an antigen binding domain specific for CD22.

In some embodiments of the nucleic acid molecules encoding a CAR of the disclosure, the CAR comprises an antigen binding domain specific for FLT3 and an antigen binding domain specific for CD33.

In some embodiments of the nucleic acid molecules encoding a CAR of the disclosure, the CAR comprises an antigen binding domain specific for FLT3 and an antigen binding domain specific for CD123.

In some embodiments of the nucleic acid molecules encoding a CAR of the disclosure, the CAR comprises an antigen binding domain specific for FLT3 and an antigen binding domain specific for Lewis-Y.

In some embodiments of the nucleic acid molecules encoding a CAR of the disclosure, the CAR comprises an antigen binding domain specific for FLT3 and an antigen binding domain specific for CD44v6.

In some embodiments of the nucleic acid molecules encoding a CAR of the disclosure, the CAR comprises an antigen binding domain specific for FLT3 and an antigen binding domain specific for CLL-1.

In some embodiments of the nucleic acid molecules encoding a CAR of the disclosure, the CAR comprises an antigen binding domain specific for FLT3 and an antigen binding domain specific for folate receptor-beta.

In some embodiments of the nucleic acid molecules encoding a CAR of the disclosure, the CAR comprises an antigen binding domain specific for FLT3 and an antigen binding domain specific for CD13.

In some embodiments of the nucleic acid molecules encoding a CAR of the disclosure, the CAR comprises an antigen binding domain specific for FLT3 and an antigen binding domain specific for CD15.

In some embodiments of the nucleic acid molecules encoding a CAR of the disclosure, the CAR comprises an antigen binding domain specific for FLT3 and an antigen binding domain specific for CD30.

In some embodiments of the nucleic acid molecules encoding a CAR of the disclosure, the CAR comprises an antigen binding domain specific for FLT3 and an antigen binding domain specific for CD45.

In some embodiments of the nucleic acid molecules encoding a CAR of the disclosure, the CAR comprises an antigen binding domain specific for FLT3 and an antigen binding domain specific for CD47.

In some embodiments of the nucleic acid molecules encoding a CAR of the disclosure, the CAR comprises an antigen binding domain specific for FLT3 and an antigen binding domain specific for Ang-2.

In some embodiments of the nucleic acid molecules encoding a CAR of the disclosure, the CAR comprises an antigen binding domain specific for FLT3 and an antigen binding domain specific for CD133.

In some embodiments of the nucleic acid molecules encoding a CAR of the disclosure, the CAR comprises an antigen binding domain specific for FLT3 and an antigen binding domain specific for WT1.

In some embodiments of the nucleic acid molecules encoding a CAR of the disclosure, the CAR comprises an antigen binding domain specific for FLT3 and an antigen binding domain specific for VEGF-A.

In some embodiments of the nucleic acid molecules encoding a CAR of the disclosure, the CAR comprises an antigen binding domain specific for FLT3 and an antigen binding domain specific for U5 snRNP200.

In some embodiments of the nucleic acid molecules encoding a CAR of the disclosure, the CAR comprises an antigen binding domain specific for FLT3 and an antigen binding domain specific for ADGRE2.

In some embodiments of the nucleic acid molecules encoding a CAR of the disclosure, the CAR comprises an antigen binding domain specific for FLT3 and an antigen binding domain specific for CD38.

In some embodiments of the nucleic acid molecules encoding a CAR of the disclosure, the CAR comprises an antigen binding domain specific for FLT3 and an antigen binding domain specific for CD157.

In some embodiments of the nucleic acid molecules encoding a CAR of the disclosure, the CAR comprises an antigen binding domain specific for FLT3 and an antigen binding domain specific for LILRB2.

In some embodiments of the nucleic acid molecules encoding a CAR of the disclosure, the CAR comprises an antigen binding domain specific for FLT3 and an antigen binding domain specific for CCR-1.

In some embodiments of the nucleic acid molecules encoding a CAR of the disclosure, the CAR comprises an antigen binding domain specific for FLT3 and an antigen binding domain specific for C-KIT.

In some embodiments of the nucleic acid molecules encoding a CAR of the disclosure, the CAR comprises an antigen binding domain specific for FLT3 and an antigen binding domain specific for CD43.

In some embodiments of the nucleic acid molecules encoding a CAR of the disclosure, the nucleic acid molecule further comprises at least one costimulatory domain or a sequence encoding at least one costimulatory domain.

In some embodiments of the nucleic acid molecules encoding a CAR of the disclosure, the antigen binding domain specific for FLT3 or a sequence encoding the antigen binding domain specific for FLT3 comprises the sequences of one or more of SEQ ID NO: 6, SEQ ID NO: 8, SEQ ID NO: 10, SEQ ID NO: 14, SEQ ID NO: 16, and SEQ ID NO: 18. In some embodiments, the antigen binding domain specific for FLT3 or a sequence encoding the antigen binding domain specific for FLT3 comprises the sequences of SEQ ID NO: 6, SEQ ID NO: 8, SEQ ID NO: 10, SEQ ID NO: 14, SEQ ID NO: 16, and SEQ ID NO: 18.

The disclosure provides a composition comprising a nucleic acid molecule of the disclosure, including a nucleic acid molecule of the disclosure encoding a CAR of the disclosure.

The disclosure provides a pharmaceutical composition comprising a nucleic acid molecule of the disclosure, including a nucleic acid molecule of the disclosure encoding a CAR of the disclosure, and a pharmaceutically acceptable carrier.

The disclosure provides a vector comprising a nucleic acid molecule of the disclosure, including a nucleic acid molecule of the disclosure encoding a CAR of the disclosure. In some embodiments, the disclosure provides a recombinant vector comprising a nucleic acid molecule of the disclosure, including a nucleic acid molecule of the disclosure encoding a CAR of the disclosure. In some embodiments, the disclosure provides a recombinant expression vector comprising a nucleic acid molecule of the disclosure, including a nucleic acid molecule of the disclosure encoding a CAR of the disclosure. In some embodiments, the disclosure provides a viral vector comprising a nucleic acid molecule of the disclosure, including a nucleic acid molecule of the disclosure encoding a CAR of the disclosure. In some embodiments, the disclosure provides a retroviral viral vector comprising a nucleic acid molecule of the disclosure, including a nucleic acid molecule of the disclosure encoding a CAR of the disclosure. In some embodiments, the viral vector is formulated for administration to an immune cell, including a T cell. In some embodiments, the viral vector is a lentiviral vector.

The disclosure provides a composition comprising a vector of the disclosure, including a viral vector of the disclosure.

The disclosure provides a pharmaceutical composition comprising vector of the disclosure, including a viral vector of the disclosure, and a pharmaceutically acceptable carrier. In some embodiments, the viral vector is a retroviral vector comprising the nucleic acid molecule of the disclosure comprising a CAR of the disclosure. In some embodiments, the viral vector is a lentiviral vector.

The disclosure provides a cell comprising a nucleic acid molecule of the disclosure, including a nucleic acid molecule encoding a CAR of the disclosure.

The disclosure provides a plurality or a population of cells, of which at least one cell comprises a nucleic acid molecule of the disclosure, including a nucleic acid molecule encoding a CAR of the disclosure. In some embodiments, the disclosure provides a plurality or a population of cells, of which a portion of the plurality of cells or a portion of the population of cells comprises a nucleic acid molecule of the disclosure, including a nucleic acid molecule encoding a CAR of the disclosure. In some embodiments, the portion is 1%, 5%, 10%, 15%, 20%, 25%, 30%, 35%, 40%, 45%, 50%, 55%, 60%, 65%, 70%, 75%, 80%, 85%, 90%, 95%, 97%, 99% or any percentage in between of the plurality or population of cells. In some embodiments, the disclosure provides a plurality or a population of cells, each of which comprises a nucleic acid molecule of the disclosure, including a nucleic acid molecule encoding a CAR of the disclosure.

The disclosure provides a cell comprising a vector of the disclosure including a viral vector of the disclosure.

The disclosure provides a plurality or a population of cells, of which at least one cell comprises a vector of the disclosure, including a viral vector of the disclosure. In some embodiments, the disclosure provides a plurality or a population of cells, of which a portion of the plurality of cells or a portion of the population of cells comprises a vector of the disclosure, including a viral vector of the disclosure. In some embodiments, the portion is 1%, 5%, 10%, 15%, 20%, 25%, 30%, 35%, 40%, 45%, 50%, 55%, 60%, 65%, 70%, 75%, 80%, 85%, 90%, 95%, 97%, 99% or any percentage in between of the plurality or population of cells. In some embodiments, the disclosure provides a plurality or a population of cells, each of which comprises a vector of the disclosure, including a viral vector of the disclosure.

The disclosure provides a cell comprising a composition of the disclosure including a pharmaceutical composition of the disclosure.

The disclosure provides a plurality or a population of cells, of which at least one cell comprises a composition of the disclosure, including a pharmaceutical composition of the disclosure. In some embodiments, the disclosure provides a plurality or a population of cells, of which a portion of the plurality of cells or a portion of the population of cells comprises a composition of the disclosure, including a pharmaceutical composition of the disclosure. In some embodiments, the portion is 1%, 5%, 10%, 15%, 20%, 25%, 30%, 35%, 40%, 45%, 50%, 55%, 60%, 65%, 70%, 75%, 80%, 85%, 90%, 95%, 97%, 99% or any percentage in between of the plurality or population of cells. In some embodiments, the disclosure provides a plurality or a population of cells, each of which comprises a composition of the disclosure, including a pharmaceutical composition of the disclosure.

In some embodiments of the cells of the disclosure, the cell is an immune cell. In some embodiments, the cell is a T-cell. In some embodiments, the cell is a natural killer (NK) cell.

In some embodiments of the plurality of cells or population of cells of the disclosure, at least one cell of the plurality or of the population is an immune cell. In some embodiments, at least one cell of the plurality or of the population is a T-cell. In some embodiments, at least one cell of the plurality or of the population is a natural killer (NK) cell.

In some embodiments of the plurality of cells or population of cells of the disclosure, a portion of the plurality or of the population comprises immune cells. In some embodiments, a portion of the plurality or of the population comprises T-cells. In some embodiments, a portion of the plurality or of the population comprises natural killer (NK) cells. In some embodiments, the portion is 1%, 5%, 10%, 15%, 20%, 25%, 30%, 35%, 40%, 45%, 50%, 55%, 60%, 65%, 70%, 75%, 80%, 85%, 90%, 95%, 97%, 99% or any percentage in between of the plurality or population of cells.

In some embodiments of the plurality of cells or population of cells of the disclosure, each cell is an immune cell. In some embodiments, each cell is a T-cell. In some embodiments, each cell is a natural killer (NK) cell.

In some embodiments of the cells of the disclosure, the cells or a plurality or a population thereof are provided or administered in an amount effective to treat cancer in a subject.

The disclosure provides a method for treating cancer, comprising: administering to a subject a therapeutically effective amount of a nucleic acid molecule of the disclosure encoding a CAR of the disclosure, wherein the cancer expresses FLT3. In some embodiments, the CAR of the disclosure comprises an antigen binding domain specific for FLT3, an antigen binding domain specific for a second antigen, a transmembrane domain, and an intracellular T cell signaling domain.

The disclosure provides a method for treating cancer, comprising: administering to a subject a therapeutically effective amount of a vector of the disclosure, wherein the cancer expresses FLT3. In some embodiments, the vector is a viral vector. In some embodiments, the vector comprises a nucleic acid molecule of the disclosure encoding a CAR of the disclosure.

The disclosure provides a nucleic acid molecule comprising: a sequence encoding a first CAR comprising a sequence encoding an antigen binding domain with specificity for FLT3 and a sequence encoding a first intracellular T cell signaling domain, and a sequence encoding a second CAR comprising a sequence encoding an antigen binding domain with specificity for a second antigen and a sequence encoding a second intracellular T cell signaling domain. In some embodiments, the nucleic acid molecule further comprises a sequence encoding a cleavable domain. In some embodiments, the sequence encoding the cleavable domain is positioned between the sequence encoding the first CAR and the sequence encoding the second CAR.

The disclosure provides a nucleic acid molecule encoding: a first CAR comprising an antigen binding domain with specificity for FLT3 and an intracellular T cell signaling domain, and a second CAR comprising an antigen binding domain with specificity for a second antigen and an intracellular T cell signaling domain. In some embodiments, the nucleic acid molecule further comprises a sequence encoding a cleavable domain. In some embodiments, the sequence encoding the cleavable domain is positioned between a sequence encoding the first CAR and a sequence encoding the second CAR.

In some embodiments of the nucleic acid molecules of the disclosure, including those wherein the nucleic acid molecule encodes a first CAR comprising an antigen binding domain with specificity for FLT3 and a second CAR comprising an antigen binding domain with specificity for a second antigen, the second antigen is selected from the group consisting of CD antigen 19 (CD19), CD antigen 22 (CD22), CD antigen 33 (CD33), CD antigen 123 (CD123), Lewis-Y, CD antigen 44, isoform 6 (CD44v6), C-type lectin-like molecule 1 (CLL-1), folate receptor-beta (FOLR2), CD antigen 13 (CD13), CD antigen 15 (CD15), CD antigen 30 (CD30), CD antigen 45 (CD45), CD antigen 47 (CD47), Angiopoietin-2 (Ang-2) CD antigen 133 (CD133), Wilms tumor protein (WT1), Vascular endothelial growth factor A (VEGF-A), U5 small nuclear ribonucleoprotein 200 kDa helicase (U5 snRNP200), Adhesion G protein-coupled receptor E2 (ADGRE2), CD antigen 38 (CD38), CD antigen 157 (CD157) Leukocyte immunoglobulin-like receptor subfamily B member 2 (LILRB2), C-C chemokine receptor type 1 (CCR-1), Proto-oncogene C-KIT, and CD antigen 43 (CD43).

In some embodiments of the nucleic acid molecules of the disclosure, including those wherein the nucleic acid molecule encodes a first CAR comprising an antigen binding domain with specificity for FLT3 and a second CAR comprising an antigen binding domain with specificity for a second antigen, the second antigen comprises CD19.

In some embodiments of the nucleic acid molecules of the disclosure, including those wherein the nucleic acid molecule encodes a first CAR comprising an antigen binding domain with specificity for FLT3 and a second CAR comprising an antigen binding domain with specificity for a second antigen, the second antigen comprises CD22.

In some embodiments of the nucleic acid molecules of the disclosure, including those wherein the nucleic acid molecule encodes a first CAR comprising an antigen binding domain with specificity for FLT3 and a second CAR comprising an antigen binding domain with specificity for a second antigen, the second antigen comprises CD33.

In some embodiments of the nucleic acid molecules of the disclosure, including those wherein the nucleic acid molecule encodes a first CAR comprising an antigen binding domain with specificity for FLT3 and a second CAR comprising an antigen binding domain with specificity for a second antigen, the second antigen comprises CD123.

In some embodiments of the nucleic acid molecules of the disclosure, including those wherein the nucleic acid molecule encodes a first CAR comprising an antigen binding domain with specificity for FLT3 and a second CAR comprising an antigen binding domain with specificity for a second antigen, the second antigen comprises CD44v6.

In some embodiments of the nucleic acid molecules of the disclosure, including those wherein the nucleic acid molecule encodes a first CAR comprising an antigen binding domain with specificity for FLT3 and a second CAR comprising an antigen binding domain with specificity for a second antigen, the second antigen comprises CLL-1.

In some embodiments of the nucleic acid molecules of the disclosure, including those wherein the nucleic acid molecule encodes a first CAR comprising an antigen binding domain with specificity for FLT3 and a second CAR comprising an antigen binding domain with specificity for a second antigen, the second antigen comprises folate receptor-beta.

In some embodiments of the nucleic acid molecules of the disclosure, including those wherein the nucleic acid molecule encodes a first CAR comprising an antigen binding domain with specificity for FLT3 and a second CAR comprising an antigen binding domain with specificity for a second antigen, the second antigen comprises CD13.

In some embodiments of the nucleic acid molecules of the disclosure, including those wherein the nucleic acid molecule encodes a first CAR comprising an antigen binding domain with specificity for FLT3 and a second CAR comprising an antigen binding domain with specificity for a second antigen, the second antigen comprises CD15.

In some embodiments of the nucleic acid molecules of the disclosure, including those wherein the nucleic acid molecule encodes a first CAR comprising an antigen binding domain with specificity for FLT3 and a second CAR comprising an antigen binding domain with specificity for a second antigen, the second antigen comprises CD30.

In some embodiments of the nucleic acid molecules of the disclosure, including those wherein the nucleic acid molecule encodes a first CAR comprising an antigen binding domain with specificity for FLT3 and a second CAR comprising an antigen binding domain with specificity for a second antigen, the second antigen comprises CD45.

In some embodiments of the nucleic acid molecules of the disclosure, including those wherein the nucleic acid molecule encodes a first CAR comprising an antigen binding domain with specificity for FLT3 and a second CAR comprising an antigen binding domain with specificity for a second antigen, the second antigen comprises CD47.

In some embodiments of the nucleic acid molecules of the disclosure, including those wherein the nucleic acid molecule encodes a first CAR comprising an antigen binding domain with specificity for FLT3 and a second CAR comprising an antigen binding domain with specificity for a second antigen, the second antigen comprises Ang-2.

In some embodiments of the nucleic acid molecules of the disclosure, including those wherein the nucleic acid molecule encodes a first CAR comprising an antigen binding domain with specificity for FLT3 and a second CAR comprising an antigen binding domain with specificity for a second antigen, the second antigen comprises CD133.

In some embodiments of the nucleic acid molecules of the disclosure, including those wherein the nucleic acid molecule encodes a first CAR comprising an antigen binding domain with specificity for FLT3 and a second CAR comprising an antigen binding domain with specificity for a second antigen, the second antigen comprises WT1.

In some embodiments of the nucleic acid molecules of the disclosure, including those wherein the nucleic acid molecule encodes a first CAR comprising an antigen binding domain with specificity for FLT3 and a second CAR comprising an antigen binding domain with specificity for a second antigen, the second antigen comprises VEGF-A.

In some embodiments of the nucleic acid molecules of the disclosure, including those wherein the nucleic acid molecule encodes a first CAR comprising an antigen binding domain with specificity for FLT3 and a second CAR comprising an antigen binding domain with specificity for a second antigen, the second antigen comprises U5 snRNP200.

In some embodiments of the nucleic acid molecules of the disclosure, including those wherein the nucleic acid molecule encodes a first CAR comprising an antigen binding domain with specificity for FLT3 and a second CAR comprising an antigen binding domain with specificity for a second antigen, the second antigen comprises ADGRE2.

In some embodiments of the nucleic acid molecules of the disclosure, including those wherein the nucleic acid molecule encodes a first CAR comprising an antigen binding domain with specificity for FLT3 and a second CAR comprising an antigen binding domain with specificity for a second antigen, the second antigen comprises CD38.

In some embodiments of the nucleic acid molecules of the disclosure, including those wherein the nucleic acid molecule encodes a first CAR comprising an antigen binding domain with specificity for FLT3 and a second CAR comprising an antigen binding domain with specificity for a second antigen, the second antigen comprises CD157.

In some embodiments of the nucleic acid molecules of the disclosure, including those wherein the nucleic acid molecule encodes a first CAR comprising an antigen binding domain with specificity for FLT3 and a second CAR comprising an antigen binding domain with specificity for a second antigen, the second antigen comprises LILRB2.

In some embodiments of the nucleic acid molecules of the disclosure, including those wherein the nucleic acid molecule encodes a first CAR comprising an antigen binding domain with specificity for FLT3 and a second CAR comprising an antigen binding domain with specificity for a second antigen, the second antigen comprises CCR-1.

In some embodiments of the nucleic acid molecules of the disclosure, including those wherein the nucleic acid molecule encodes a first CAR comprising an antigen binding domain with specificity for FLT3 and a second CAR comprising an antigen binding domain with specificity for a second antigen, the second antigen comprises C-KIT.

In some embodiments of the nucleic acid molecules of the disclosure, including those wherein the nucleic acid molecule encodes a first CAR comprising an antigen binding domain with specificity for FLT3 and a second CAR comprising an antigen binding domain with specificity for a second antigen, the second antigen comprises CD43.

In some embodiments of the nucleic acid molecules of the disclosure, including those wherein the nucleic acid molecule encodes a first CAR comprising an antigen binding domain with specificity for FLT3 and a second CAR comprising an antigen binding domain with specificity for a second antigen, the first CAR further comprises at least one costimulatory domain, and wherein the second CAR further comprises at least one costimulatory domain.

In some embodiments of the nucleic acid molecules of the disclosure, including those wherein the nucleic acid molecule encodes a first CAR comprising an antigen binding domain with specificity for FLT3 and a second CAR comprising an antigen binding domain with specificity for a second antigen, the antigen binding domain specific for FLT3 comprises the sequences of SEQ ID NO: 6, SEQ ID NO: 8, SEQ ID NO: 10, SEQ ID NO: 14, SEQ ID NO: 16, and SEQ ID NO: 18.

The disclosure provides a vector comprising a nucleic acid molecules of the disclosure, including those comprising a sequence encoding a first CAR comprising a sequence encoding an antigen binding domain with specificity for FLT3 and a sequence encoding a first intracellular T cell signaling domain, and a sequence encoding a second CAR comprising a sequence encoding an antigen binding domain with specificity for a second antigen and a sequence encoding a second intracellular T cell signaling domain. In some embodiments, the vector is a recombinant vector. In some embodiments, the vector is an expression vector. In some embodiments, the vector is a recombinant expression vector. In some embodiments, the vector is a viral vector. In some embodiments, the vector is a retroviral vector. In some embodiments, the vector is a lentiviral vector. In some embodiments, the vector is formulated for administration to an immune cell, including a T cell.

The disclosure provides a cell comprising a nucleic acid molecule of the disclosure, including a nucleic acid molecule comprising a sequence encoding a first CAR comprising a sequence encoding an antigen binding domain with specificity for FLT3 and a sequence encoding a first intracellular T cell signaling domain, and a sequence encoding a second CAR comprising a sequence encoding an antigen binding domain with specificity for a second antigen and a sequence encoding a second intracellular T cell signaling domain.

The disclosure provides a cell comprising a vector of the disclosure, including a vector comprising a nucleic acid molecule comprising a sequence encoding a first CAR comprising a sequence encoding an antigen binding domain with specificity for FLT3 and a sequence encoding a first intracellular T cell signaling domain, and a sequence encoding a second CAR comprising a sequence encoding an antigen binding domain with specificity for a second antigen and a sequence encoding a second intracellular T cell signaling domain.

The disclosure comprises a composition comprising a nucleic acid molecule of the disclosure, including a nucleic acid molecule comprising a sequence encoding a first CAR comprising a sequence encoding an antigen binding domain with specificity for FLT3 and a sequence encoding a first intracellular T cell signaling domain, and a sequence encoding a second CAR comprising a sequence encoding an antigen binding domain with specificity for a second antigen and a sequence encoding a second intracellular T cell signaling domain. In some embodiments, the composition further comprises a pharmaceutically-acceptable carrier (i.e. a pharmaceutical composition).

The disclosure comprises a composition comprising a vector of the disclosure, including those comprising a nucleic acid molecule comprising a sequence encoding a first CAR comprising a sequence encoding an antigen binding domain with specificity for FLT3 and a sequence encoding a first intracellular T cell signaling domain, and a sequence encoding a second CAR comprising a sequence encoding an antigen binding domain with specificity for a second antigen and a sequence encoding a second intracellular T cell signaling domain. In some embodiments, the composition further comprises a pharmaceutically-acceptable carrier (i.e. a pharmaceutical composition).

The disclosure comprises a composition comprising a cell of the disclosure, including those comprising a nucleic acid molecule comprising a sequence encoding a first CAR comprising a sequence encoding an antigen binding domain with specificity for FLT3 and a sequence encoding a first intracellular T cell signaling domain, and a sequence encoding a second CAR comprising a sequence encoding an antigen binding domain with specificity for a second antigen and a sequence encoding a second intracellular T cell signaling domain or those comprising a vector comprising the nucleic acid molecule. In some embodiments, the composition further comprises a pharmaceutically-acceptable carrier (i.e. a pharmaceutical composition).

The disclosure comprises a composition comprising a plurality of cells or a population of cells of the disclosure. In some embodiments, the composition further comprises a pharmaceutically-acceptable carrier (i.e. a pharmaceutical composition).

The disclosure provides a cell population comprising a first cell and a second cell, wherein the first cell comprises a first nucleic acid molecule and the second cell comprises a second nucleic acid molecule, wherein the first nucleic acid molecule encodes a first CAR comprising an antigen binding domain with specificity for FLT3 and an intracellular T cell signaling domain, wherein the second nucleic acid molecule encodes a second CAR comprising an antigen binding domain with specificity for a second antigen and an intracellular T cell signaling domain. In some embodiments, the first cell or the second cell is an immune cell. In some embodiments, the first cell and the second cell are immune cells. In some embodiments, the first cell or the second cell is a T cell. In some embodiments, the first cell and the second cell are T-cells. In some embodiments, the first cell or the second cell is a NK cell. In some embodiments, the first cell and the second cell are NK cells.

The disclosure provides a cell population comprising a first cell and a second cell, wherein the first cell comprises a first nucleic acid molecule and the second cell comprises a second nucleic acid molecule, wherein the first nucleic acid molecule encodes a first CAR comprising an antigen binding domain with specificity for FLT3 and an intracellular T cell signaling domain, wherein the second nucleic acid molecule encodes a second CAR comprising an antigen binding domain with specificity for a second antigen and an intracellular T cell signaling domain. In some embodiments, the second antigen is selected from the group consisting of CD antigen 19 (CD19), CD antigen 22 (CD22), CD antigen 33 (CD33), CD antigen 123 (CD123), Lewis-Y, CD antigen 44, isoform 6 (CD44v6), C-type lectin-like molecule 1 (CLL-1), folate receptor-beta (FOLR2), CD antigen 13 (CD13), CD antigen 15 (CD15), CD antigen 30 (CD30), CD antigen 45 (CD45), CD antigen 47 (CD47), Angiopoietin-2 (Ang-2) CD antigen 133 (CD133), Wilms tumor protein (WT1), Vascular endothelial growth factor A (VEGF-A), U5 small nuclear ribonucleoprotein 200 kDa helicase (U5 snRNP200), Adhesion G protein-coupled receptor E2 (ADGRE2), CD antigen 38 (CD38), CD antigen 157 (CD157) Leukocyte immunoglobulin-like receptor subfamily B member 2 (LILRB2), C-C chemokine receptor type 1 (CCR-1), Proto-oncogene C-KIT, and CD antigen 43 (CD43).

The disclosure provides an immune cell population comprising a first immune cell and a second immune cell, wherein the first immune cell comprises a first nucleic acid molecule and the second immune cell comprises a second nucleic acid molecule, wherein the first nucleic acid molecule encodes a first CAR comprising an antigen binding domain with specificity for FLT3 and an intracellular T cell signaling domain, wherein the second nucleic acid molecule encodes a second CAR comprising an antigen binding domain with specificity for a second antigen and an intracellular T cell signaling domain. In some embodiments, the second antigen is selected from the group consisting of CD antigen 19 (CD19), CD antigen 22 (CD22), CD antigen 33 (CD33), CD antigen 123 (CD123), Lewis-Y, CD antigen 44, isoform 6 (CD44v6), C-type lectin-like molecule 1 (CLL-1), folate receptor-beta (FOLR2), CD antigen 13 (CD13), CD antigen 15 (CD15), CD antigen 30 (CD30), CD antigen 45 (CD45), CD antigen 47 (CD47), Angiopoietin-2 (Ang-2) CD antigen 133 (CD133), Wilms tumor protein (WT1), Vascular endothelial growth factor A (VEGF-A), U5 small nuclear ribonucleoprotein 200 kDa helicase (U5 snRNP200), Adhesion G protein-coupled receptor E2 (ADGRE2), CD antigen 38 (CD38), CD antigen 157 (CD157) Leukocyte immunoglobulin-like receptor subfamily B member 2 (LILRB2), C-C chemokine receptor type 1 (CCR-1), Proto-oncogene C-KIT, and CD antigen 43 (CD43).

In some embodiments of the immune cell populations of the disclosure, including those wherein the first immune cell comprises a first nucleic acid molecule encoding a first CAR and the second immune cell comprises a second nucleic acid molecule encoding a second CAR, the first CAR comprises an antigen binding domain with specificity for FLT3 and the second CAR comprises an antigen binding domain with specificity for CD19.

In some embodiments of the immune cell populations of the disclosure, including those wherein the first immune cell comprises a first nucleic acid molecule encoding a first CAR and the second immune cell comprises a second nucleic acid molecule encoding a second CAR, the first CAR comprises an antigen binding domain with specificity for FLT3 and the second CAR comprises an antigen binding domain with specificity for CD22.

In some embodiments of the immune cell populations of the disclosure, including those wherein the first immune cell comprises a first nucleic acid molecule encoding a first CAR and the second immune cell comprises a second nucleic acid molecule encoding a second CAR, the first CAR comprises an antigen binding domain with specificity for FLT3 and the second CAR comprises an antigen binding domain with specificity for CD33.

In some embodiments of the immune cell populations of the disclosure, including those wherein the first immune cell comprises a first nucleic acid molecule encoding a first CAR and the second immune cell comprises a second nucleic acid molecule encoding a second CAR, the first CAR comprises an antigen binding domain with specificity for FLT3 and the second CAR comprises an antigen binding domain with specificity for CD123.

In some embodiments of the immune cell populations of the disclosure, including those wherein the first immune cell comprises a first nucleic acid molecule encoding a first CAR and the second immune cell comprises a second nucleic acid molecule encoding a second CAR, the first CAR comprises an antigen binding domain with specificity for FLT3 and the second CAR comprises an antigen binding domain with specificity for Lewis-Y.

In some embodiments of the immune cell populations of the disclosure, including those wherein the first immune cell comprises a first nucleic acid molecule encoding a first CAR and the second immune cell comprises a second nucleic acid molecule encoding a second CAR, the first CAR comprises an antigen binding domain with specificity for FLT3 and the second CAR comprises an antigen binding domain with specificity for CD44v6.

In some embodiments of the immune cell populations of the disclosure, including those wherein the first immune cell comprises a first nucleic acid molecule encoding a first CAR and the second immune cell comprises a second nucleic acid molecule encoding a second CAR, the first CAR comprises an antigen binding domain with specificity for FLT3 and the second CAR comprises an antigen binding domain with specificity for CLL-1.

In some embodiments of the immune cell populations of the disclosure, including those wherein the first immune cell comprises a first nucleic acid molecule encoding a first CAR and the second immune cell comprises a second nucleic acid molecule encoding a second CAR, the first CAR comprises an antigen binding domain with specificity for FLT3 and the second CAR comprises an antigen binding domain with specificity for folate receptor-beta.

In some embodiments of the immune cell populations of the disclosure, including those wherein the first immune cell comprises a first nucleic acid molecule encoding a first CAR and the second immune cell comprises a second nucleic acid molecule encoding a second CAR, the first CAR comprises an antigen binding domain with specificity for FLT3 and the second CAR comprises an antigen binding domain with specificity for CD13.

In some embodiments of the immune cell populations of the disclosure, including those wherein the first immune cell comprises a first nucleic acid molecule encoding a first CAR and the second immune cell comprises a second nucleic acid molecule encoding a second CAR, the first CAR comprises an antigen binding domain with specificity for FLT3 and the second CAR comprises an antigen binding domain with specificity for CD15.

In some embodiments of the immune cell populations of the disclosure, including those wherein the first immune cell comprises a first nucleic acid molecule encoding a first CAR and the second immune cell comprises a second nucleic acid molecule encoding a second CAR, the first CAR comprises an antigen binding domain with specificity for FLT3 and the second CAR comprises an antigen binding domain with specificity for CD30.

In some embodiments of the immune cell populations of the disclosure, including those wherein the first immune cell comprises a first nucleic acid molecule encoding a first CAR and the second immune cell comprises a second nucleic acid molecule encoding a second CAR, the first CAR comprises an antigen binding domain with specificity for FLT3 and the second CAR comprises an antigen binding domain with specificity for CD45.

In some embodiments of the immune cell populations of the disclosure, including those wherein the first immune cell comprises a first nucleic acid molecule encoding a first CAR and the second immune cell comprises a second nucleic acid molecule encoding a second CAR, the first CAR comprises an antigen binding domain with specificity for FLT3 and the second CAR comprises an antigen binding domain with specificity for CD47.

In some embodiments of the immune cell populations of the disclosure, including those wherein the first immune cell comprises a first nucleic acid molecule encoding a first CAR and the second immune cell comprises a second nucleic acid molecule encoding a second CAR, the first CAR comprises an antigen binding domain with specificity for FLT3 and the second CAR comprises an antigen binding domain with specificity for Ang-2.

In some embodiments of the immune cell populations of the disclosure, including those wherein the first immune cell comprises a first nucleic acid molecule encoding a first CAR and the second immune cell comprises a second nucleic acid molecule encoding a second CAR, the first CAR comprises an antigen binding domain with specificity for FLT3 and the second CAR comprises an antigen binding domain with specificity for CD133.

In some embodiments of the immune cell populations of the disclosure, including those wherein the first immune cell comprises a first nucleic acid molecule encoding a first CAR and the second immune cell comprises a second nucleic acid molecule encoding a second CAR, the first CAR comprises an antigen binding domain with specificity for FLT3 and the second CAR comprises an antigen binding domain with specificity for WT1.

In some embodiments of the immune cell populations of the disclosure, including those wherein the first immune cell comprises a first nucleic acid molecule encoding a first CAR and the second immune cell comprises a second nucleic acid molecule encoding a second CAR, the first CAR comprises an antigen binding domain with specificity for FLT3 and the second CAR comprises an antigen binding domain with specificity for VEGF-A.

In some embodiments of the immune cell populations of the disclosure, including those wherein the first immune cell comprises a first nucleic acid molecule encoding a first CAR and the second immune cell comprises a second nucleic acid molecule encoding a second CAR, the first CAR comprises an antigen binding domain with specificity for FLT3 and the second CAR comprises an antigen binding domain with specificity for U5 snRNP200.

In some embodiments of the immune cell populations of the disclosure, including those wherein the first immune cell comprises a first nucleic acid molecule encoding a first CAR and the second immune cell comprises a second nucleic acid molecule encoding a second CAR, the first CAR comprises an antigen binding domain with specificity for FLT3 and the second CAR comprises an antigen binding domain with specificity for ADGRE2.

In some embodiments of the immune cell populations of the disclosure, including those wherein the first immune cell comprises a first nucleic acid molecule encoding a first CAR and the second immune cell comprises a second nucleic acid molecule encoding a second CAR, the first CAR comprises an antigen binding domain with specificity for FLT3 and the second CAR comprises an antigen binding domain with specificity for CD38.

In some embodiments of the immune cell populations of the disclosure, including those wherein the first immune cell comprises a first nucleic acid molecule encoding a first CAR and the second immune cell comprises a second nucleic acid molecule encoding a second CAR, the first CAR comprises an antigen binding domain with specificity for FLT3 and the second CAR comprises an antigen binding domain with specificity for CD157.

In some embodiments of the immune cell populations of the disclosure, including those wherein the first immune cell comprises a first nucleic acid molecule encoding a first CAR and the second immune cell comprises a second nucleic acid molecule encoding a second CAR, the first CAR comprises an antigen binding domain with specificity for FLT3 and the second CAR comprises an antigen binding domain with specificity for LILRB2.

In some embodiments of the immune cell populations of the disclosure, including those wherein the first immune cell comprises a first nucleic acid molecule encoding a first CAR and the second immune cell comprises a second nucleic acid molecule encoding a second CAR, the first CAR comprises an antigen binding domain with specificity for FLT3 and the second CAR comprises an antigen binding domain with specificity for CCR-1.

In some embodiments of the immune cell populations of the disclosure, including those wherein the first immune cell comprises a first nucleic acid molecule encoding a first CAR and the second immune cell comprises a second nucleic acid molecule encoding a second CAR, the first CAR comprises an antigen binding domain with specificity for FLT3 and the second CAR comprises an antigen binding domain with specificity for C-KIT.

In some embodiments of the immune cell populations of the disclosure, including those wherein the first immune cell comprises a first nucleic acid molecule encoding a first CAR and the second immune cell comprises a second nucleic acid molecule encoding a second CAR, the first CAR comprises an antigen binding domain with specificity for FLT3 and the second CAR comprises an antigen binding domain with specificity for CD43.

In some embodiments of the immune cell populations of the disclosure, including those wherein the first immune cell comprises a first nucleic acid molecule encoding a first CAR and the second immune cell comprises a second nucleic acid molecule encoding a second CAR, first CAR further comprises at least one costimulatory domain, and wherein the second CAR further comprises at least one costimulatory domain. The disclosure provides a composition comprising an immune cell population of the disclosure. In some embodiments, the composition further comprises a pharmaceutically acceptable carrier (i.e. a pharmaceutical composition).

The disclosure provides a method for treating cancer, comprising: administering to a subject a cell of the disclosure, wherein the cancer expresses FLT3. In some embodiments, the cell comprises a nucleic acid molecule of the disclosure encoding a CAR of the disclosure. In some embodiments, the CAR of the disclosure comprises an antigen binding domain specific for FLT3, an antigen binding domain specific for a second antigen, a transmembrane domain, and an intracellular T cell signaling domain. In some embodiments, the cell comprises a vector comprising a nucleic acid molecule of the disclosure encoding a CAR of the disclosure. In some embodiments, the cell comprises a composition comprising a vector or a nucleic acid molecule of the disclosure. In some embodiments, a plurality or a population of cells of the disclosure are administered to the subject.

The disclosure provides a method for treating cancer, comprising: administering to a subject a therapeutically effective amount, concentration or dose of a plurality of cells or a population of cells of the disclosure, wherein the cancer expresses FLT3.

The disclosure provides a method for treating cancer, comprising: administering to a subject a therapeutically effective amount of a composition of the disclosure, wherein the cancer expresses FLT3. In some embodiments, the composition comprises one or more of a nucleic acid molecule of the disclosure encoding a CAR of the disclosure, a vector of the disclosure or a cell of the disclosure. In some embodiments, the composition further comprises a pharmaceutically-acceptable carrier (i.e. the composition is a pharmaceutical composition).

In some embodiments of the methods of treating cancer of the disclosure, the cancer expresses FLT3 because a proliferating cell expresses FLT3. In some embodiments, the cancer expresses FLT3 because one or more tumor cells express FLT3. In some embodiments, the cancer expresses FLT3 because malignant cells express FLT3. In some embodiments, the cancer expresses FLT3 because one or more metastatic cells express FLT3.

In some embodiments of the methods of treating cancer of the disclosure, the cancer further expresses a second antigen. In some embodiments, a proliferating cell expresses FLT3 and a second antigen. In some embodiments, one or more tumor cells express FLT3 and a second antigen. In some embodiments, malignant cells express FLT3 and a second antigen. In some embodiments, one or more metastatic cells express FLT3 and a second antigen. In some embodiments, the second antigen is selected from the group consisting of CD antigen 19 (CD19), CD antigen 22 (CD22), CD antigen 33 (CD33), CD antigen 123 (CD123), Lewis-Y, CD antigen 44, isoform 6 (CD44v6), C-type lectin-like molecule 1 (CLL-1), folate receptor-beta (FOLR2), CD antigen 13 (CD13), CD antigen 15 (CD15), CD antigen 30 (CD30), CD antigen 45 (CD45), CD antigen 47 (CD47), Angiopoietin-2 (Ang-2) CD antigen 133 (CD133), Wilms tumor protein (WT1), Vascular endothelial growth factor A (VEGF-A), U5 small nuclear ribonucleoprotein 200 kDa helicase (U5 snRNP200), Adhesion G protein-coupled receptor E2 (ADGRE2), CD antigen 38 (CD38), CD antigen 157 (CD157) Leukocyte immunoglobulin-like receptor subfamily B member 2 (LILRB2), C-C chemokine receptor type 1 (CCR-1), Proto-oncogene C-KIT, and CD antigen 43 (CD43).

In some embodiments of the methods of treating cancer of the disclosure, the cancer expresses FLT3 and CD19.

In some embodiments of the methods of treating cancer of the disclosure, the cancer expresses FLT3 and CD22.

In some embodiments of the methods of treating cancer of the disclosure, the cancer expresses FLT3 and CD33.

In some embodiments of the methods of treating cancer of the disclosure, the cancer expresses FLT3 and CD123

In some embodiments of the methods of treating cancer of the disclosure, the cancer expresses FLT3 and Lewis-Y.

In some embodiments of the methods of treating cancer of the disclosure, the cancer expresses FLT3 and CD44v6.

In some embodiments of the methods of treating cancer of the disclosure, the cancer expresses FLT3 and CLL-1.

In some embodiments of the methods of treating cancer of the disclosure, the cancer expresses FLT3 and folate receptor-beta.

In some embodiments of the methods of treating cancer of the disclosure, the cancer expresses FLT3 and CD13.

In some embodiments of the methods of treating cancer of the disclosure, the cancer expresses FLT3 and CD15.

In some embodiments of the methods of treating cancer of the disclosure, the cancer expresses FLT3 and CD30.

In some embodiments of the methods of treating cancer of the disclosure, the cancer expresses FLT3 and CD45.

In some embodiments of the methods of treating cancer of the disclosure, the cancer expresses FLT3 and CD47.

In some embodiments of the methods of treating cancer of the disclosure, the cancer expresses FLT3 and Ang-2.

In some embodiments of the methods of treating cancer of the disclosure, the cancer expresses FLT3 and CD133.

In some embodiments of the methods of treating cancer of the disclosure, the cancer expresses FLT3 and WT1.

In some embodiments of the methods of treating cancer of the disclosure, the cancer expresses FLT3 and VEGF-A).

In some embodiments of the methods of treating cancer of the disclosure, the cancer expresses FLT3 and U5 snRNP200).

In some embodiments of the methods of treating cancer of the disclosure, the cancer expresses FLT3 and ADGRE2.

In some embodiments of the methods of treating cancer of the disclosure, the cancer expresses FLT3 and CD38.

In some embodiments of the methods of treating cancer of the disclosure, the cancer expresses FLT3 and CD157.

In some embodiments of the methods of treating cancer of the disclosure, the cancer expresses FLT3 and LILRB2.

In some embodiments of the methods of treating cancer of the disclosure, the cancer expresses FLT3 and CCR-1.

In some embodiments of the methods of treating cancer of the disclosure, the cancer expresses FLT3 and C-KIT.

In some embodiments of the methods of treating cancer of the disclosure, the cancer expresses FLT3 and CD43.

### BRIEF DESCRIPTION OF THE DRAWINGS

FIG. 1 is a diagram of nucleic acid molecules encoding bispecific CARs in accordance with embodiments of the disclosure.

FIG. 2 is a diagram of nucleic acid molecules encoding bicistronic CARs in accordance with embodiments of the disclosure.

FIG. 3 is a diagram of nucleic acid molecules encoding monospecific CARs. In some embodiments of the disclosure, a population of immune cells comprises a first cell comprising a first CAR and a second cell comprising a second CAR, wherein the first CAR has an antigen binding domain with specificity for FLT3 and the second CAR has an antigen binding domain with specificity for a second antigen, e.g., CD19.

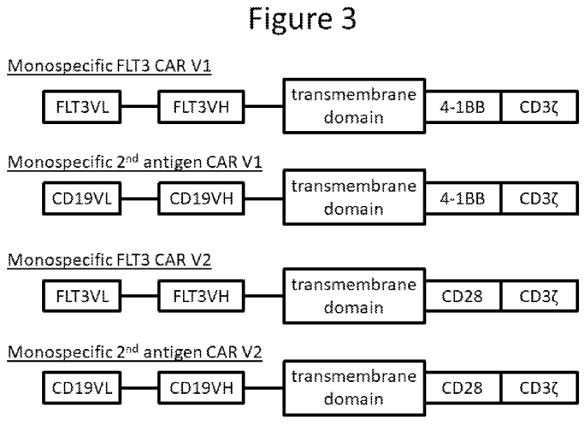

### DETAILED DESCRIPTION

Acute Lymphoblastic Leukemia (ALL) and Acute Myeloid Leukemia (AML) represent common oncologic diagnoses in children. Substantial progress has been made in the upfront chemotherapy for pediatric ALL and AML such that most patients will be cured. Nonetheless, ALL remains a common cause of death from cancer in children due to relapse of disease that no longer responds to cytotoxic chemotherapy, or due to refractories to upfront treatment. Furthermore, long-term therapy-induced morbidity remains a major issue, particularly those patients deemed to be high-risk for relapse and thus treated with more intense regimens under current risk-adapted protocols. In adults, ALL occurs less commonly than in children, but the prognosis for adult ALL is worse than in children undergoing standard cytotoxic chemotherapy. Treatment of young adults on pediatric—type regimens has improved outcomes but not to the level achieved in children.

The adoptive cell transfer (ADT or ACT) of T cells genetically modified to express chimeric antigen receptors (CARs) targeting antigens expressed on lymphoid cells have demonstrated potent activity in B cell malignancies, including ALL, resulting in remissions in chemotherapy refractory patients. The surface protein being targeted in the majority of these trials is the CD19 antigen that is expressed on both malignant and non-malignant B cells. However, not all patients respond, and relapses occur, in some cases due to loss of CD19 expression.

Patients with infant ALL or AML express high levels of FMS-like tyrosine kinase 3 (FLT3). FLT3 is also known as Fms-Related Tyrosine Kinase 3, Stem Cell Tyrosine Kinase 1, FL Cytokine Receptor, CD135 Antigen, FLK-2, STK1, and Fetal Liver Kinase 2. FLT3 is frequently mutated in AML, causing activation of the pathway, and is thought to be a major driver of disease. Thus, down-modulation of FLT3 will be an improbable escape mechanism. Additionally, the mutations are found in the intracellular domain of the receptor so immune cells expressing FLT3 CARs will be able to target both wild type and mutant forms of FLT3 allowing for broad targeting of both infant ALL and AML and may target any FLT3-overexpressing leukemia.

In some embodiments, the disclosure provides a chimeric antigen receptor (CAR) comprising an antigen binding domain specific for FLT3, an antigen binding domain specific for a second antigen, a transmembrane domain, and an intracellular T cell signaling domain. The CAR may further comprise a 4-1BB intracellular domain, a linker, a spacer, or a combination thereof. In some embodiments, the two or more antigen binding domains may be arranged in tandem and separated by linker sequences. Further embodiments of the disclosure provide a construct comprising (a) a cleavable domain; (b) a first CAR comprising a first antigen binding domain, a first transmembrane domain, and a first intracellular T cell signaling domain; and (c) a second CAR comprising a second antigen binding domain, a second transmembrane domain, and a second intracellular T cell signaling domain; wherein the first and second CARs are linked through the cleavable domain, wherein when the first CAR is cleaved from the construct, the first antigen binding domain has antigenic specificity for FLT3. Further embodiments of the disclosure provide a population comprising a first cell comprising a first CAR with an antigen binding domain specific for FLT3, a transmembrane domain, and an intracellular T cell signaling domain, and a second cell comprising a second CAR with an antigen binding domain specific for a second antigen, a transmembrane domain, and an intracellular T cell signaling domain.

A chimeric antigen receptor (CAR) is an artificially constructed hybrid protein or polypeptide containing the antigen binding domain of an antibody (e.g., single chain variable fragment (scFv)) linked to T-cell signaling domains. Characteristics of CARs include their ability to redirect T-cell specificity and reactivity toward at least one selected target in a non-MHC-restricted manner, exploiting the antigen-binding properties of monoclonal antibodies. The non-MHC-restricted antigen recognition gives T cells expressing CARs the ability to recognize an antigen independent of antigen processing, thus bypassing a major mechanism of tumor escape. Moreover, when expressed in T− cells, CARs advantageously do not dimerize with endogenous T cell receptor (TCR) alpha and beta chains. A bispecific CAR is specific to two different antigens. In some embodiments, the CAR of the disclosure is a bispecific CAR.

In some embodiments of the disclosure, the disclosure provides multiple CARs (e.g., two, three, four, five, or more) that each bind to at least one antigen, wherein each CAR is separated by a cleavable domain. In some embodiments of the disclosure, cleaving the cleavable domain releases each CAR, e.g., a first and second CAR, from the CAR construct such that each cleaved CAR is separately present on the T cell surface. Each CAR has antigenic specificity for its respective target, and each can elicit an antigen-specific response. In some embodiments, such a CAR construct can have two CARs cleaved/released, e.g., a bicistronic CAR. Without wishing to be bound by theory or mechanism, the cleavable domains of these CARs may be cleaved after full translation of the full sequence or after translation of each CAR and cleavable domain, such that a CAR is cleaved/released prior to translation of the next CAR in the sequence.

In some embodiments, the disclosure provides a population of cells expressing a first CAR with an antigen binding domain specific for FLT3 and a second CAR with an antigen binding domain specific for a second antigen. The first CAR and second CAR may be expressed by the same cell (as in a bicistronic CAR T cell) or different cells. Additionally, the first CAR and second CAR may be expressed by the same cell type or different cell types. For instance, in some embodiments, the cell expressing a FLT3 CAR is a CD4+ T cell and the cell expressing a CAR specific for a second antigen, e.g., a CD19 CAR, is a CD8+ T cell, or the cell expressing a FLT3 CAR is a CD8+ T cell and the cell expressing a CD19 CAR is a CD4+ T cell. In other embodiments, the cell expressing a FLT3 CAR is a T cell and the cell expressing a CD19 CAR is a NK cell, or the cell expressing a FLT3 CAR is a NK cell and the cell expressing a CD19 CAR is a T cell. In other embodiments, the cell expressing a FLT3 CAR and the cell expressing a CD19 CAR are both NK cells or are both T cells, e.g., are both CD4+ T cells, or are both CD8+ T cells. The first CAR and second CAR can comprise the same or different intracellular signaling domains. Without wishing to be bound by theory or mechanism, it is believed that a population of cells expressing a CAR specific for FLT3 and a CAR specific for a second antigen on the same or different cells may have greater therapeutic effect than a population of cells expressing one monospecific CAR. A population of cells expressing a CAR specific for FLT3 and a CAR specific for a second antigen may reduce or prevent cancer cell escape or relapse due to heterogeneous levels of target antigen expression or loss of target antigen expression.

In some embodiments, the disclosure provides a first CAR comprising an antigen binding domain with specificity for FLT3, and a second CAR comprising an antigen binding domain with specificity for a second antigen. In some embodiments, the second antigen is selected from the group consisting of CD antigen 19 (CD19), CD antigen 22 (CD22), CD antigen 33 (CD33), CD antigen 123 (CD123), Lewis-Y, CD antigen 44, isoform 6 (CD44v6), C-type lectin-like molecule 1 (CLL-1), folate receptor-beta (FOLR2), CD antigen 13 (CD13), CD antigen 15 (CD15), CD antigen 30 (CD30), CD antigen 45 (CD45), CD antigen 47 (CD47), Angiopoietin-2 (Ang-2) CD antigen 133 (CD133), Wilms tumor protein (WT1), Vascular endothelial growth factor A (VEGF-A), U5 small nuclear ribonucleoprotein 200 kDa helicase (U5 snRNP200), Adhesion G protein-coupled receptor E2 (ADGRE2), CD antigen 38 (CD38), CD antigen 157 (CD157) Leukocyte immunoglobulin-like receptor subfamily B member 2 (LILRB2), C-C chemokine receptor type 1 (CCR-1), Proto-oncogene C-KIT, and CD antigen 43 (CD43).

The CAR constructs or cell populations of the disclosure may provide many advantages. In some embodiments of the disclosure, for example, the CAR constructs or cell populations of the disclosure may, advantageously, reduce or prevent cancer cell escape due to loss of expression of one antigen, e.g., FLT3 or CD19, by the cancer cell. For example, it is believed that the CAR constructs or cell populations of the disclosure may reduce or prevent relapses that have been observed in cancer patients following treatment with a CAR having antigenic specificity for a single antigen and whose cancer has lost expression of that antigen. Also, the CAR constructs or cell populations of the disclosure may be advantageous for treating patients who have heterogeneous levels of expression of FLT3, a second antigen, or both. The CAR constructs of the disclosure may increase the patient population that may be successfully treated. For example, a patient that may fail to respond to a CAR therapy that targets only FLT3 may respond to a CAR therapy that targets CD19, and a patient that may fail to respond to a CAR therapy that targets only CD19 may respond to a CAR therapy that targets FLT3. In another example, a patients' cancer may have heterogeneous levels of expression of FLT3 and a second antigen, e.g., CD19, such that monospecific CAR therapy targeting only FLT3 or only CD19 would be ineffective or would result in a relapse, but embodiments of the present disclosure targeting both FLT3 and the second antigen would provide a more effective treatment or would prevent relapse. Additionally, regarding the cleavable or bicistronic CARs, co-transduction of T cells using two vectors, each having a single CAR, provides a relatively low percentage of cells expressing both CARs and substantial numbers of T cells expressing one or the other CAR only; where this is desirable, an advantage of using the cleavable CAR constructs is that there may be equal or substantially equal expression of each CAR in each T cell that successfully integrates the construct. Similarly, a T cell that successfully integrates a bispecific CAR construct will be capable of responding to both antigens. By targeting both FLT3 and a second antigen, the cleavable and non-cleavable CAR constructs, e.g. a bispecific CAR construct, or the cell populations, may, advantageously, provide synergistic responses as compared to therapies which target only a single antigen, and may also provide a more broadly active therapy to patients with heterogeneous expression of one or both of FLT3 and a second antigen on cancer cells.

Thus, without being bound to a particular theory or mechanism, it is believed that by eliciting an antigen-specific response against two antigens, e.g., FLT3 and CD19, the CAR constructs or cell populations provide for one or more of any of the following: targeting and destroying FLT3-expressing cancer cells, targeting and destroying cancer cells expressing a second antigen, e.g., CD19, reducing or eliminating cancer cells, facilitating infiltration of immune cells to tumor site(s), and enhancing/extending anti-cancer responses.

In some embodiments, the disclosure provides a CAR comprising an antigen binding domain specific for FLT3, based on antibodies, e.g., NC7. NC7 is described in U.S. Pat. No. 8,071,099, which is incorporated herein by reference in its entirety. The scFv comprises a light chain variable region and a heavy chain variable region. In some embodiments of the disclosure, the light chain and heavy chain may comprise any suitable combination of light chain and heavy chain sequences, e.g., as listed in Table 2 below.

The CARs of the disclosure may target several (such as two or more, three or more) different antigens. In some embodiments, the CAR is a bispecific CAR and targets FLT3 and at least one additional antigen. In some embodiments, the additional antigen is selected from the group consisting of CD antigen 19 (CD19), CD antigen 22 (CD22), CD antigen 33 (CD33), CD antigen 123 (CD123), Lewis-Y, CD antigen 44, isoform 6 (CD44v6), C-type lectin-like molecule 1 (CLL-1), folate receptor-beta (FOLR2), CD antigen 13 (CD13), CD antigen 15 (CD15), CD antigen 30 (CD30), CD antigen 45 (CD45), CD antigen 47 (CD47), Angiopoietin-2 (Ang-2) CD antigen 133 (CD133), Wilms tumor protein (WT1), Vascular endothelial growth factor A (VEGF-A), U5 small nuclear ribonucleoprotein 200 kDa helicase (U5 snRNP200), Adhesion G protein-coupled receptor E2 (ADGRE2), CD antigen 38 (CD38), CD antigen 157 (CD157) Leukocyte immunoglobulin-like receptor subfamily B member 2 (LILRB2), C-C chemokine receptor type 1 (CCR-1), Proto-oncogene C-KIT, and CD antigen 43 (CD43).

As described herein, the antigen binding domains of the CAR may be arranged in tandem and may be separated by linker peptides. The antigens targeted by the CAR may be antigens on a single cell (such as a cancerous B-cell) or antigens that are expressed on separate cells that each contribute to a disease. The antigens targeted by the CAR are antigens which are either directly or indirectly involved in a disease.

In a bispecific CAR, at least two different antigen-specific antibodies or fragments thereof or derivatives thereof may be cloned into antigen binding domains. The antibodies may be specific for any, but at least two, distinct antigens of choice. The antibody specific to the antigen may be the Fab fragment of the antibody or the single chain variable fragment (scFv) of the antibody.

In some embodiments of the disclosure, scFvs specific to FLT3 and at least one additional antigen may be cloned upstream (i.e., to N-terminus) of the costimulatory domain(s) so long as the target-antigens are expressed on cells that are targetable by the genetically modified cells described herein. Such techniques are explained fully in the literature. (Sambrook et al, “Molecular Cloning: A Laboratory Manual” (1989), Current Protocols in Molecular Biology. Volumes I-III [Ausubel, R. M., ed. (1994)], Cell Biology: A Laboratory Handbook. Volumes I-III [J. E. Celis, ed. (1994))], Current Protocols in Immunology. Volumes I-III [Coligan, J. E., ed. (1994)], Oligonucleotide Synthesis. (M. J. Gait ed. 1984), Nucleic Acid Hybridization [B. D. Hames & S. J. Higgins eds. (1985)], Transcription And Translation [B. D. Hames & S. J. Higgins, eds. (1984)], Animal Cell Culture [R. I. Freshney, ed. (1986)], Immobilized Cells And Enzymes [IRL Press, (1986)], Practical Guide To Molecular Cloning B. Perbal (1984), Current Protocols in Immunology (J. E. Coligan, A. M. Kruisbeek, D. H. Margulies, E. M. Shevach and W. Strober, eds., 1991), Annual Review of Immunology as well as monographs in journals such as Advances in Immunology).

In one embodiment, each antigen binding domain comprises the full-length IgG heavy chain (specific for the target antigen) having the V_{H}, CH1, hinge, and the CH2 and CH3 (Fc) Ig domains, if the V_{H} domain alone is sufficient to confer antigen-specificity (single-domain antibodies). The full length IgG heavy chain may be linked to the co-stimulatory domain and the intracellular signaling domain via the appropriate transmembrane domain. If both, the V_{H} and the V_{L} domains are necessary to generate a fully active antigen binding domain, the V_{H}-containing CAR and the full-length lambda light chain (IgL) are both introduced into the cells to generate an active antigen binding domain. In some embodiments, a spacer domain may be linked between the antigen binding domain(s) and the transmembrane domain.

In another embodiment, the extracellular portion of the CAR comprises at least two single chain antibody variable fragments (scFv), each specific for a different target antigen. scFvs, in which the C-terminus of one variable domain (V_{H} or V_{L}) is tethered to the N-terminus of the other (V_{L} or V_{H}, respectively) via a polypeptide linker, have been developed without significantly disrupting antigen binding or specificity of the binding. (Chaudhary et al., A recombinant single-chain immunotoxin composed of anti-Tac variable regions and a truncated diphtheria toxin. 1990 Proc. Natl. Acad. Sci., 87:9491; Bedzyk et al. Immunological and structural characterization of a high affinity anti-fluorescein single-chain antibody. 1990 J. Biol. Chem., 265:18615). A linker connects the N-terminus of the V_{H} with the C-terminus of V_{L} or the C-terminus of V_{H} with the N-terminus of V_{L}. These scFvs lack the constant regions (Fc) present in the heavy and light chains of the native antibody. The scFvs, specific for at least two different antigens, are arranged in tandem and linked to the co-stimulatory domain and the intracellular signaling domain via a transmembrane domain. In some embodiments, a spacer domain may be linked between the antigen binding domain(s) and the transmembrane domain.

In some embodiments, each scFv fragment may be fused to all or a portion of the constant domains of the heavy chain. The resulting antigen binding domain, specific for at least two different antigens, is joined to the co-stimulatory domain and the intracellular signaling domain via a transmembrane domain. In some embodiments, a spacer domain may be linked between the antigen binding domain and the transmembrane domain.

In some embodiments, each antigen binding domain of the CAR comprises a divalent (or bivalent) single-chain variable fragment (di-scFvs, bi-scFvs). In CARs comprising di-scFvs, two scFvs specific for each antigen are linked together by producing a single peptide chain with two V_{H} and two V_{L} regions, yielding tandem scFvs. (Xiong, Cheng-Yi; Nataraj an, A; Shi, X B; Denardo, G L; Denardo, S J (2006). “Development of tumor targeting anti-MUC-1 multimer: effects of di-scFv unpaired cysteine location on PEGylation and tumor binding”. Protein Engineering Design and Selection 19 (8): 359-367; Kufer, Peter; Lutterbuse, Ralf; Baeuerle, Patrick A. (2004). “A revival of bispecific antibodies”. Trends in Biotechnology 22 (5): 238-244). CARs comprising at least two antigen binding domains would express two scFvs specific for each of the two antigens. The resulting antigen binding domain, specific for at least two different antigens, is joined to the co-stimulatory domain and the intracellular signaling domain via a transmembrane domain. In some embodiments, a spacer domain may be linked between the antigen binding domain(s) and the transmembrane domain.

In an additional embodiment, each antigen binding domain of the CAR comprises a diabody. In a diabody, the scFvs are created with linker peptides that are too short for the two variable regions to fold together, driving the scFvs to dimerize. Still shorter linkers (one or two amino acids) lead to the formation of trimers, the so-called triabodies or tribodies. Tetrabodies may also be used.

To create bispecific CARs of the present disclosure, two or more individual antigen binding domains are connected to each other, either covalently or noncovalently, on a single protein molecule. An oligo- or polypeptide linker, an Fc hinge or membrane hinge region may be used to connect these domains to each other. The CARs of the present disclosure may comprise two or more of the different antigen binding domains connected together in different combinations. For example, two or more antigen binding domains containing immunoglobulin sequences (e.g. scFvs and/or single-domain antibodies) may be linked to each other.

In some embodiments, the antigen binding domain comprises a linker, which links together any of the domains or regions of the CAR of the disclosure. In a preferred embodiment, the linker connects the heavy chain variable region and the light chain variable region of the antigen binding domain. Any linker suitable for linking domains of the CAR, especially the heavy chain variable region and the light chain variable region may be used. In some embodiments, the linker comprises, consists of, or consists essentially of a glycine-serine linker domain. Preferably, the antigen binding domain comprises a scFv comprising a heavy chain variable region, a light chain variable region, and a linker. In some embodiments of the disclosure, the light chain, heavy chain, and linker may comprise any suitable combination of light chain, heavy chain, and linker sequences as listed in Table 2 below. In some embodiments, the CAR comprises more than one linker.

The first CAR and the second CAR of a bicistronic CAR construct are joined to each other through 1, 2, 3, 4 or more cleavable domains. The cleavable domain(s) may comprise one or more of any suitable cleavable domain, including domains recognized by cleavage enzymes or domains that are self-cleaving. Suitable domains include, for example, the 2A domain, such as T2A and/or P2A, and furin cleavage sequences. Table 1 presents exemplary suitable cleavable domains. The GSG residues in Table 1 may be added to improve cleavage efficiency.

TABLE 1

| | SEQ | |
|---|---|---|
| Sequence | ID NO: | Notes |
| (GSG)EGRGSLLTCGDVEENPGP | 31 | T2A |
| (GSG)ATNFSLLKQAGDVEENPGP | 32 | P2A |
| (GSG)QCTNYALLKLAGDVESNPGP | 33 | E2A |
| (GSG)VKQTLNFDLLKLAGDVESNPGP | 34 | F2A |
| RKRR | 35 | Furin |

In some embodiments of the disclosure, the CAR construct contains more than one cleavable domain, wherein the cleavable domains are all the same. In some embodiments of the disclosure, the CAR construct contains more than one cleavable domain adjacent within the CAR construct, wherein at least one cleavable domain is different.

In some embodiments of the disclosure, the CAR comprises, consists of, or consists essentially of the sequence:

| (SEQ ID NO: 1) | |
|---|---|
| 1 | MLLVTSLLLC ELPHPAFLLI PEVQLVQSGA EVKKPGSSVK VSCKASGGTF SSYAISWVRQ |
| 61 | APGQGLEWMG GIIPIFGTAN YAQKFQGRVT ITADKSTSTA YMELSSLRSE DTAVYYCATF |
| 121 | ALFGFREQAF DIWGQGTTVT VSSGGGGSGG GGSGGGGSDI QMTQSPSSLS ASVGDRVTIT |
| 181 | CRASQSISSY LNWYQOKPGK APKLLIYAAS SLQSGVPSRF SGSGSGTDFT LTISSLQPED |
| 241 | LATYYCOOSY STPFTFGPGT KVDIKSGTTT RAPRPPTPAP TIASQPLSLR PEACRPAAGG |
| 301 | AVHTRGLDFA CDIYIWAPLA GTCGVLLLSL VITLYCKRGR KKLLYIFKQP FMRPVQTTQE |
| 361 | EDGCSCRFPE EEEGGCELRV KFSRSADAPA YKQGQNQLYN ELNLGRREEY DVLDKRRGRD |
| 421 | PEMGGKPRRK NPQEGLYNEL QKDKMAEAYS EIGMKGERRR GKGHDGLYQG LSTATKDTYD |
| 481 | ALHMQALPPR. |

In some embodiments of the disclosure, the CAR comprises, consists of, or consists essentially of the sequence:

| (SEQ ID NO: 2) | |
|---|---|
| 1 | MLLVTSLLLC ELPHPAFILI PEVQLVOSGA EVKKPGSSVK VSCKASGGTF SSYAISWVRQ |
| 61 | APGQGLEWMG GIIPIFGTAN YAQKFQGPVT ITADKSTSTA YMELSSLRSE DTAVYYCATF |
| 121 | ALFGFREQAF DIWGQGTTVT VSSGGGGSGG GGSGGGGSDI QMTQSPSSLS ASVGDRVTIT |
| 181 | CRASQSISSY LNWYQQKPGK APKLLIYAAS SLQSGVPSRF SGSGSGTDFT LTISSLQPED |
| 241 | LATYYCQQSY STPFTFGPGT KVDIKSGLED PAEPKSPDKT HTCPPCPAPE LLGGPSVFLF |
| 301 | PPKPKDTLMI SRTPEVTCVV VDVSHEDPEV KFNWYVDGVE VHNAKTKPRE EQYNSTYRVV |
| 361 | SVLTVLHQDW LNGKEYKCKV SNKALPAPIE KTISKAKGQP REPQVYTLPP SRDELTKNQV |
| 421 | SLTCLVKGFY PSDIAVEWES NGQPENNYKT TPPVLDSDGS FFLYSKLTVD KSRWQQGNVF |
| 481 | SCSVMHEALH NHYTQKSLSL SPGKKDPKTT TPAPRPPTPA PTIASQPLSL RPEACRPAAG |
| 541 | GAVHTRGLDF ACDIYIWAPL AGTCGVLLLS LVITLYCKRG RKKLLYIFKQ PFMRPVQTTQ |
| 601 | EEDGCSCRFP EEEEGGCELR VKFSRSADAP AYKQGQNQLY NELNLGRREE YDVLDKRRGR |
| 661 | DPEMGGKPRR KNPQEGLYNE LQKDKMAEAY SEIGMKGERR RGKGHDGLYQ GLSTATKDTY |
| 721 | DALHMQALPP R. |

In some embodiments, the antigen binding domain comprises a leader/signal sequence. The leader sequence may be positioned at the amino terminus of the heavy chain variable region. The leader sequence may comprise any suitable leader sequence. In some embodiments of the disclosure, the leader/signal sequence may comprise the sequence as listed in Table 2 below. In the mature form of the T cell, the leader sequence may not be present.

In some embodiments of the disclosure, the CAR comprises, consists of, or consists essentially of the sequence:

| (SEQ ID NO: 29) | |
|---|---|
| 1 | EVQLVQSGAE VKKPGSSVKV SCKASGGTFS SYAISWVRQA PGQGLEWMGG IIPIFGTANY |
| 61 | AQKFQGRVTI TADKSTSTAY MELSSLRSED TAVYYCATFA LFGFREQAFD IWGQGTTVTV |
| 121 | SSGGGGSGGG GSGGGGSDIQ MTQSPSSLSA SVGDRVTITC RASQSISSYL NWYQQKPGKA |
| 181 | PKLLIYAASS LQSGVPSRFS GSGSGTDFTL TISSLQPEDL ATYYCQQSYS TPFTFGPGTK |
| 241 | VDIKSGTTTP APRPPTPAPT IASQPLSLRP EACRPAAGGA VHTRGLDFAC DIYIWAPLAG |
| 301 | TCGVLLLSLV ITLYCKRGRK KLLYIFKQPF MRPVQTTQEE DGCSCRFPEE EEGGCELRVK |
| 361 | FSRSADAPAY KQGQNQLYNE LNLGRREEYD VLDKRRGRDP EMGGKPRRKN PQEGLYNELQ |
| 421 | KDKMAEAYSE IGMKGERRRG KGHDGLYQGL STATKDTYDA LHMQALPPR. |

In some embodiments of the disclosure, the CAR comprises, consists of, or consists essentially of the sequence:

| (SEQ ID NO: 30) | |
|---|---|
| 1 | EVQLVQSGAE VKKPGSSVKV SCKASGGTFS SYAISWVRQA PGQGLEWMGG IIPIFGTANY |
| 61 | AQKFQGRVTI TADKSTSTAY MELSSLRSED TAVYYCATFA LFGFREQAFD IWGQGTTVTV |
| 121 | SSGGGGSGGG GSGGGGSDIQ MTQSPSSLSA SVGDRVTITC RASQSISSYL NWYQQKPGKA |
| 181 | PKLLIYAASS LQSGVPSRFS GSGSGTDFTL TISSLQPEDL ATYYCQQSYS TPFTFGPGTK |
| 241 | VDIKSGLEDP AEPKSPDKTH TCPPCPAPEL LGGPSVFLFP PKPKDTLMIS RTPEVTCVVV |
| 301 | DVSHEDPEVK FNWYVDGVEV HNAKTKPREE QYNSRVVSVL TVLHQDWLNG KEYKCKVSNK |
| 361 | ALPAPIEKTI SKAKGQPREP QVYTLPPSRD ELTKNQVSLT CLVKGFYPSD IAVEWESNGQ |
| 421 | PENNYKTTPP VLDSDGSFFL YSKLTVDKSR WQQGNVFSCS VMHEALHNHY TQKSLSLSPG |
| 481 | KKDPKTTTPA PRPPTPAPTI ASQPLSLRPE ACRPAAGGAV HTRGLDFACD IYIWAPLAGT |
| 541 | CGVLLLSLVI TLYCKRGRKK LLYIFKQPFM RPVQTTQEED GCSCRFPEEE EGGCELRVKF |
| 601 | SRSADAPAYK QGQNQLYNEL NLGRREEYDV LDKRRGRDPE MGGKPRRKNP QEGLYNELQK |
| 661 | DKMAEAYSEI GMKGERRREK GHDGLYQGLS TATKDTYDAL HMQALPPR. |

In some embodiments of the disclosure, the CAR comprises a transmembrane domain. In some embodiments of the disclosure, the transmembrane domain comprises CD8. The CD8 can comprise the CD8a (CD8 alpha) hinge and transmembrane domain. In a preferred embodiment, the CD8 is human. The CD8 may comprise less than the whole CD8. In some embodiments of the disclosure, the CD8 may comprise the sequence as listed in Table 2 below.

In some embodiments of the disclosure, the CAR comprises an intracellular T cell signaling domain comprising 4-1BB (CD137), CD3 zeta (ζ), or both. In a preferred embodiment, the CD3 zeta, 4-1BB, or both is/are human. 4-1BB transmits a potent costimulatory signal to T cells, promoting differentiation and enhancing long-term survival of T lymphocytes, CD3ζ associates with TCRs to produce a signal and contains immunoreceptor tyrosine-based activation motifs (ITAMs). In some embodiments, the CAR lacks a 4-1BB domain. In another embodiment, the CAR comprises a CD28 domain. CD28 is a T cell marker important in T cell co-stimulation. The 4-1BB, CD28, CD3 zeta, or any of these may comprise less than the whole 4-1BB, CD28 or CD3 zeta, respectively. In some embodiments of the disclosure, the 4-1BB may comprise the sequence as listed in Table 2 below. In some embodiments of the disclosure, the CD3 zeta may comprise the sequence as listed in Table 2 below.

In some embodiments of the disclosure, the CAR comprises a spacer. The spacer may be between any aforementioned domains. In some embodiments, the CAR comprises an IgG heavy chain constant domain (CH2CH3) spacer. In some embodiments, the spacer can be between the scFv and the transmembrane domain. In a preferred embodiment, the sequence of the spacer, e.g., CH2CH3, is human. In some embodiments of the disclosure, the spacer may comprise the sequence as listed in Table 2 below. In some embodiments, the spacer comprises a hydrophilic region and facilitates proper protein folding. In some embodiments, a spacer is optional. In such a CAR, the antigen binding domains are at the N-terminus, arranged in tandem, and separated by a linker peptide.

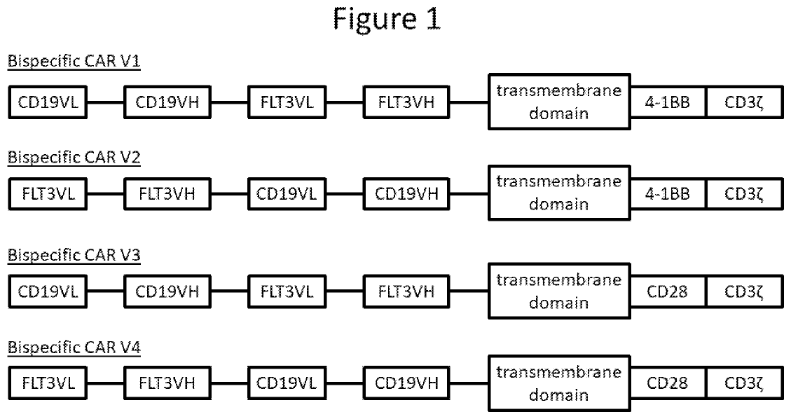

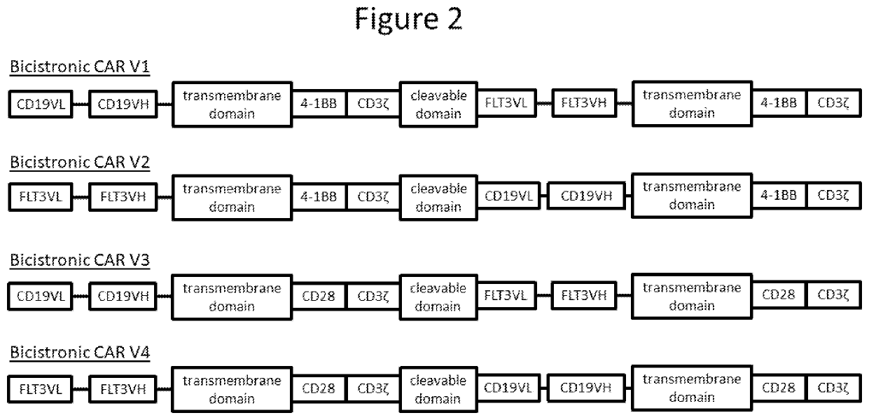

The disclosure provides nucleic acid molecules which are exemplary embodiments of the disclosure where the CAR is specific to FLT3 and CD19. In one embodiment, the CAR is a bispecific anti-FLT3×CD19 CAR as encoded by a nucleic acid molecule set forth in FIG. 1. In one embodiment, the CAR is a bicistronic anti-FLT3×CD19 CAR as encoded by a nucleic acid molecule set forth in FIG. 2. In some embodiments, the bispecific CAR comprises scFvs specific for FLT3 and CD19 with each scFv separated by a linker, and collectively joined to the co-stimulatory and intracellular signaling domains via a transmembrane domain. Any scFv specific for FLT3 and CD19 may be used, and CD19 may be replaced by any second antigen. In some embodiments, the second antigen is selected from the group consisting of CD antigen 19 (CD19), CD antigen 22 (CD22), CD antigen 33 (CD33), CD antigen 123 (CD123), Lewis-Y, CD antigen 44, isoform 6 (CD44v6), C-type lectin-like molecule 1 (CLL-1), folate receptor-beta (FOLR2), CD antigen 13 (CD13), CD antigen 15 (CD15), CD antigen 30 (CD30), CD antigen 45 (CD45), CD antigen 47 (CD47), Angiopoietin-2 (Ang-2) CD antigen 133 (CD133), Wilms tumor protein (WT1), Vascular endothelial growth factor A (VEGF-A), U5 small nuclear ribonucleoprotein 200 kDa helicase (U5 snRNP200), Adhesion G protein-coupled receptor E2 (ADGRE2), CD antigen 38 (CD38), CD antigen 157 (CD157) Leukocyte immunoglobulin-like receptor subfamily B member 2 (LILRB2), C-C chemokine receptor type 1 (CCR-1), Proto-oncogene C-KIT, and CD antigen 43 (CD43).

The disclosure provides a nucleic acid molecule encoding a chimeric antigen receptor (CAR) comprising: an antigen binding domain specific for FLT3, an antigen binding domain specific for a second antigen, a transmembrane domain, and an intracellular T cell signaling domain. In some embodiments, the second antigen is selected from the group consisting of CD antigen 19 (CD19), CD antigen 22 (CD22), CD antigen 33 (CD33), CD antigen 123 (CD123), Lewis-Y, CD antigen 44, isoform 6 (CD44v6), C-type lectin-like molecule 1 (CLL-1), folate receptor-beta (FOLR2), CD antigen 13 (CD13), CD antigen 15 (CD15), CD antigen 30 (CD30), CD antigen 45 (CD45), CD antigen 47 (CD47), Angiopoietin-2 (Ang-2) CD antigen 133 (CD133), Wilms tumor protein (WT1), Vascular endothelial growth factor A (VEGF-A), U5 small nuclear ribonucleoprotein 200 kDa helicase (U5 snRNP200), Adhesion G protein-coupled receptor E2 (ADGRE2), CD antigen 38 (CD38), CD antigen 157 (CD157) Leukocyte immunoglobulin-like receptor subfamily B member 2 (LILRB2), C-C chemokine receptor type 1 (CCR-1), Proto-oncogene C-KIT, and CD antigen 43 (CD43).

B-lymphocyte antigen CD19 may also be known as B-lymphocyte surface antigen B4, Differentiation antigen CD1 or T-cell surface antigen Leu-12.

B-cell receptor CD22 may also be known as B-lymphocyte cell adhesion molecule (BL-CAM), Sialic acid-binding Ig-like lectin 2 (Siglec-2) or T-cell surface antigen Leu-14.

Myeloid cell surface antigen CD33 may also be known as Sialic acid-binding Ig-like lectin 3 (Siglec-3) or glycoprotein gp67.

CD123 may also be known as Interleukin-3 (IL-3) receptor subunit alpha, IL-3R subunit alpha, IL-3R-alpha, or IL-3RA.

Lewis-Y may also be known as 4-galactosyl-N-acetylglucosaminide 3-alpha-L-fucosyltransferase 9 (FUT9), Fucosyltransferase 9 (FUT-9), Fucosyltransferase IX (Fuc-TIX or FucT-IX), or Galactoside 3-L-fucosyltransferase.

CD44v6 may also be known as CDw44, Extracellular matrix receptor III (ECMR-III or GP90 lymphocyte homing/adhesion receptor), HUTCH-I, Heparan sulfate proteoglycan, Hermes antigen, Hyaluronate receptor, Phagocytic glycoprotein 1 (PGP-1), Phagocytic glycoprotein I (PGP-I).

CLL-1 may also be known as Dendritic cell-associated lectin 2 (DCAL-2), Myeloid inhibitory C-type lectin-like receptor (MICL), C-type lectin domain family 12 member A (CLEC12A), or CD antigen 371 (CD371).

Folate receptor-beta (FOLR2) may also be known as FR-beta, Folate receptor 2, Folate receptor fetal/placental, Placental folate-binding protein (FBP).

CD13 may also be known as Aminopeptidase N (AP-N or hAPN), Alanyl aminopeptidase, Aminopeptidase M (AP-M), Microsomal aminopeptidase, Myeloid plasma membrane glycoprotein CD13, or gp150.

CD30 may also be known as Tumor necrosis factor receptor superfamily member 8 (TNFRSF8), CD30L receptor, Ki-1 antigen or Lymphocyte activation antigen CD30.

CD45 may also be known as Receptor-type tyrosine-protein phosphatase C (PTPRC) or Leukocyte common antigen (L-CA or T200).

CD47 may also be known as Leukocyte surface antigen CD47, Antigenic surface determinant protein 0A3, Integrin-associated protein (IAP), or Protein MER6

CD133 may also be known as prominin-1, Antigen AC133, or Prominin-like protein 1.

WT1 may also be known as WT33.

VEGF-A may also be known as vascular permeability factor (VPF).

U5 snRNP200 may also be known as Activating signal cointegrator 1 complex subunit 3-like 1, BRR2 homolog, U5 snRNP-specific 200 kDa protein (U5-200KD).

ADGRE2 (EMR2) may also be known as EGF-like module receptor 2 (EMR2), EGF-like module-containing mucin-like hormone receptor-like 2, or CD antigen 312 (CD312).

CD38 may also be known as ADP-ribosyl cyclase/cyclic ADP-ribose hydrolase 1, 2′-phospho-ADP-ribosyl cyclase, 2′-phospho-ADP-ribosyl cyclase/2′-phospho-cyclic-ADP-ribose transferase, 2′-phospho-cyclic-ADP-ribose transferase, ADP-ribosyl cyclase 1 (ADPRC 1), Cyclic ADP-ribose hydrolase 1 (cADPr hydrolase 1), or T10.CD157 may also be known as ADP-ribosyl cyclase/cyclic ADP-ribose hydrolase 2, ADP-ribosyl cyclase 2, Bone marrow stromal antigen 1 (BST-1), Cyclic ADP-ribose hydrolase 2 (cADPr hydrolase 2).

LILRB2 may also be known as Leukocyte immunoglobulin-like receptor (LIR-2), CD85 antigen-like family member D (CD85d), Immunoglobulin-like transcript 4 (ILT-4), Monocyte/macrophage immunoglobulin-like receptor 10 (MIR-10) or CD antigen 85d (CD85d).

CCR-1 may also be known as C-C CKR-1, CC-CKR-1, CCR1, HM145, LD78 receptor. Macrophage inflammatory protein 1-alpha receptor (MIP-1alpha-R), RANTES-R, or CD antigen 191 (CD191). C-KIT may also be known as Mast/stem cell growth factor receptor Kit (SCFR), Piebald trait protein (PBT) Proto-oncogene c-Kit, Tyrosine-protein kinase Kit, p145 c-kit, v-kit Hardy-Zuckerman 4 feline sarcoma viral oncogene homolog or CD antigen 117 (CD117).

CD43 may also be known as Leukosialin, GPL115, Galactoglycoprotein (GALGP), Leukocyte sialoglycoprotein, Sialophorin or CD antigen 43 (CD43).

Embodiments of the disclosure comprise sequences as provided in Table 2 below.

TABLE 2

| | SEQ ID | | |
|---|---|---|---|
| Sequence | NO: | Segment | Notes |
| M | | | Start methionine |
| LLVTSLLLCELPHPAFLLIP | 4 | | Signal peptide: |
| | | | from human |
| | | | GM-CSF |
| EVQLVQSGAEVKKPGSSVKVSCKAS | 5 | scFv | Heavy chain: |
| | | | FR1 |
| GGTFSSYAIS | 6 | scFv | Heavy chain: |
| | | | CDR1 |
| WVRQAPGQGLEWMG | 7 | scFv | Heavy chain: |
| | | | FR2 |
| GIIPIFGTANYAQKFQG | 8 | scFv | Heavy chain: |
| | | | CDR2 |
| RVTITADKSTSTAYMELSSLRSEDTAVYYCAT | 9 | scFv | Heavy chain: |
| | | | FR3 |
| FALFGFREQAFDI | 10 | scFv | Heavy chain: J |
| | | | region (CDR3) |
| WGQGTTVTVSS | 11 | scFv | Heavy chain: |
| | | | FR4 |
| GGGGSGGGGSGGGGS | 12 | scFv | linker |
| DIQMTQSPSSLSASVGDRVTITC | 13 | scFv | Light chain: |
| | | | FR1 |
| RASQSISSYLN | 14 | scFv | Light chain: |
| | | | CDR1 |
| WYQQKPGKAPKLLIY | 15 | scFv | Light chain: |
| | | | FR2 |
| AASSLQS | 16 | scFv | Light chain: |
| | | | CDR2 |
| GVPSRFSGSGSGTDFTLTISSLQPEDLATYYC | 17 | scFv | Light chain: |
| | | | FR3 |
| QQSYSTPFT | 18 | scFv | Light chain: J |
| | | | region (CDR3) |
| FGPGTKVDIK | 19 | scFv | Light chain: |
| | | | FR4 |
| SG | | | Added amino |
| | | | acids due to |
| | | | vector design |
| LEDP | 21 | Spacer | |
| AEPKSPDKTHTCPPCPAPELLGGPSVFLFPPK | 22 | Spacer | CH2 |
| PKDTLMISRTPEVTCVVVDVSHEDPEVKFNWY | | | |
| VDGVEVHNAKTKPREEQYNSTYRVVSVLTVLH | | | |
| QDWLNGKEYKCKVSNKALPAPIEKTISKAK | | | |
| GQPREPQVYTLPPSRDELTKNQVSLTCLVKGF | 23 | Spacer | CH3 |
| YPSDIAVEWESNGQPENNYKTTPPVLDS | | | |
| DGSFFLYSKLTVDKSRWQQGNVFSCSVMHEAL | | | |
| HNHYTQKSLSLSPGK | | | |
| KDPK | 24 | Spacer | |
| TTTPAPRPPTPAPTIASQPLSLRPEACRPAAG | 25 | CD8 | CD8α hinge |
| GAVHTRGLDFACD | | | |
| IYIWAPLAGTCGVLLLSLVITLYC | 26 | CD8 | CD8α |
| | | | transmembrane |
| | | | domain |
| KRGRKKLLYIFKQPFMRPVQTTQEEDGCSCRF | 27 | 4-1BB | Intracellular |
| PEEEEGGCEL | | | domain |
| RVKFSRSADAPAYKQGQNQLYNELNLGRREEY | 28 | CD3ζ | Intracellular |
| DVLDKRRGRDPEMGGKPRRKNPQEGLYN | | | domain |
| ELQKDKMAEAYSEIGMKGERRRGKGHDGLYQG | | | |
| LSTATKDTYDALHMQALPPR | | | |

Embodiments of the disclosure include the following sequences in Table 3 that comprise the sequences presented in Table 2 above.

TABLE 3

| | Name | |
|---|---|---|
| | Short Form | Long Form |
| | SEQ ID NO: | |
| | 1 | 2 |
| Comprising Table 2 SEQ ID | Initiator Met (M) | Initiator Met (M) |
| NOS: | 4 | 4 |
| | 5 | 5 |
| | 6 | 6 |
| | 7 | 7 |
| | 8 | 8 |
| | 9 | 9 |
| | 10 | 10 |
| | 11 | 11 |
| | 12 | 12 |
| | 13 | 13 |
| | 14 | 14 |
| | 15 | 15 |
| | 16 | 16 |
| | 17 | 17 |
| | 18 | 18 |
| | 19 | 19 |
| | 20 | 20 |
| | | 21 |
| | | 22 |
| | | 23 |
| | | 24 |
| | 25 | 25 |
| | 26 | 26 |
| | 27 | 27 |
| | 28 | 28 |

Embodiments of the disclosure include the following sequences in Table 4 that comprise the sequences presented in Table 2 above, where the signal peptide is not present.

TABLE 4

| | Name | |
|---|---|---|
| | Short Form | Long Form |
| | SEQ ID NO: | |
| | 29 | 30 |
| Comprising Table 2 SEQ ID | 5 | 5 |
| NOS: | 6 | 6 |
| | 7 | 7 |
| | 8 | 8 |
| | 9 | 9 |
| | 10 | 10 |
| | 11 | 11 |
| | 12 | 12 |
| | 13 | 13 |
| | 14 | 14 |
| | 15 | 15 |
| | 16 | 16 |
| | 17 | 17 |
| | 18 | 18 |
| | 19 | 19 |
| | 20 | 20 |
| | | 21 |
| | | 22 |
| | | 23 |
| | | 24 |
| | 25 | 25 |
| | 26 | 26 |
| | 27 | 27 |
| | 28 | 28 |

In some embodiments, the CAR or CAR construct comprises an antigen binding domain specific for CD19. In some embodiments, this antigen binding domain comprises or encodes an anti-CD19 scFv or CDR described in WO2014153270A1. In some embodiments, this antigen binding domain encodes a sequence from the group: SEQ ID NO 1 through SEQ ID NO 12 of WO2014153270A1 (provided in the sequence listing as SEQ ID NOs: 36-47, respectively). In some embodiments, the CAR or CAR construct comprises a scFv specific for CD19 derived from FMC63 or SJ25C1.

In some embodiments, the CAR or CAR construct comprises an antigen binding domain specific for CD123. In some embodiments, this antigen binding domain comprises or encodes an anti-CD123 scFv or CDR described in US20180312595A1. In some embodiments, this antigen binding domain encodes a sequence from the group: SEQ ID NO 157 through SEQ ID NO 160 or SEQ ID NO 184 through SEQ ID NO 215 of US20180312595A1 (provided in the sequence listing as SEQ ID NOs: 48-51 and 52-83, respectively). In some embodiments, the CAR or CAR construct comprises an antigen binding domain specific for CD123 as described in US20190002573A1 or encoding a sequence selected from the group SEQ ID NO 54-72 of US20190002573A1 (provided in the sequence listing as SEQ ID NOs: 84-100 and 102, respectively).

In some embodiments, the CAR or CAR construct comprises an antigen binding domain specific for CD33. In some embodiments, this antigen binding domain comprises an anti-CD33 scFv or CDR described in US20180044423A1. In some embodiments, this antigen binding domain encodes a sequence from the group: SEQ ID NO: 39-47, 57-65, 66-74, or 262-268 of US20180044423A1 (provided in the sequence listing as SEQ ID NOs: 103-111, 112-129 and 130-136, respectively). In some embodiments, the CAR or CAR construct comprises a scFv specific for CD33 derived from the antibody HIM3-4.

In some embodiments, an antigen binding domain against Lewis Y is an antigen binding portion, e.g., CDRs, of an antibody described in, Kelly et al., Cancer Biother Radiopharm 23(4):411-423 (2008) (hu3S 193 Ab (scFvs)); or Dolezal et al., Protein Engineering 16(1):47-56 (2003) (NC10 scFv).

In some embodiments, the CAR or CAR construct comprises an antigen binding domain specific for CD22. In some embodiments, this antigen binding domain comprises or encodes an anti-CD22 scFv or CDR described in US20180125892A1. In some embodiments, this antigen binding domain comprises or encodes a sequence on Table 6 of US20180125892A1. In some embodiments, the CAR or CAR construct comprises a scFv specific for CD22 derived from m971 or m972.

In some embodiments, the CAR or CAR construct comprises an antigen binding domain specific for CLL1. In some embodiments, this antigen binding domain comprises or encodes an anti-CLL1 scFv or CDR described in WO2016120218A1. In some embodiments, this antigen binding domain comprises or encodes a sequence from the group: SEQ ID NO 109 through SEQ ID NO 190 of WO2016120218A1. In some embodiments, the CAR or CAR construct comprises a scFv specific for CLL1 as described in U.S. Pat. No. 8,536,310B2 or encoding a sequence selected from the group SEQ ID NO 5, 7, 9, and 11 of U.S. Pat. No. 8,536,310B2, which correspond to VH and VL of anti CLL1 antibodies 21.16 and 1075.7.

In some embodiments, the CAR or CAR construct comprises an antigen binding domain specific for folate receptor beta. In some embodiments, this antigen binding domain comprises an anti-folate receptor beta scFv or CDR described in U.S. Pat. No. 9,446,105B2. In some embodiments, this antigen binding domain comprises an anti-folate receptor beta scFv or CDR described in U.S. Pat. No. 9,446,105B2. In some embodiments, this antigen binding domain comprises a sequence from the group: SEQ ID NO 1, SEQ ID NO 3, SEQ ID NO 5, SEQ ID NO 7, SEQ ID NO 9, and SEQ ID NO 11 of U.S. Pat. No. 9,446,105B2.

In some embodiments, the CAR or CAR construct comprises an antigen binding domain specific for CD30. In some embodiments, this antigen binding domain comprises an anti-CD30 scFv or CDR described in U.S. Pat. No. 7,090,843B1. In some embodiments, this antigen binding domain encodes or comprises a sequence from the group: SEQ ID NO 1 through SEQ ID NO 32 of U.S. Pat. No. 7,090,843B1.

In some embodiments, an antigen binding domain against CD43 is an antigen binding portion, e.g., CDRs, of an antibody described in US20190016813A1. In some embodiments, this antigen binding domain encodes a sequence from the group: SEQ ID NO 110, 113, 118, 119, 120, and 125 of US20190016813A1.

In some embodiments, the CAR or CAR construct comprises an antigen binding domain specific for CD15. In some embodiments, this antigen binding domain comprises a portion of an anti-CD15 antibody described in Leukocyte typing IV (1989); Leukocyte typing II (1984); Leukocyte typing VI (1995); Solter D. et al., Proc. Natl. Acad. Sci. USA 75:5565 (1978); Kannagi R. et al., J. Biol. Chem. 257:14865 (1982); Magnani, J. L. et al., Arch. Biochem. Biophys 233:501 (1984); or Eggens I. et al., J. Biol. Chem. 264:9476 (1989).

In some embodiments, the CAR or CAR construct comprises an antigen binding domain specific for CD45. In some embodiments, this antigen binding domain comprises a CD45 scFv or CDR described in WO2018237022A1. In some embodiments, this antigen binding domain encodes SEQ ID NO 34 of WO2018237022A1.

In some embodiments, an antigen binding domain against CD47 is an antigen binding portion, e.g., CDRs, of an antibody described in U.S. Ser. No. 10/239,945B2. In some embodiments, this antigen binding domain encodes a sequence from the group: SEQ ID NO 1 through SEQ ID NO 20 of U.S. Ser. No. 10/239,945B2.

The phrases “have antigen specificity” and “elicit antigen-specific response” as used herein means that the CAR can specifically bind to and immunologically recognize an antigen, such that binding of the CAR to the antigen elicits an immune response.

Included in the scope of the disclosure are functional portions of the CARs described herein. The term “functional portion” when used in reference to a CAR refers to any part or fragment of the CAR of the disclosure, which part or fragment retains the biological activity of the CAR of which it is a part (the parent CAR). Functional portions encompass, for example, those parts of a CAR that retain the ability to recognize target cells, or detect, treat, or prevent a disease, to a similar extent, the same extent, or to a higher extent, as the parent CAR. In reference to the parent CAR, the functional portion can comprise, for instance, about 10% 25%, 30%, 50%, 68%, 80%, 90%, 95%, or more, of the parent CAR.

The functional portion can comprise additional amino acids at the amino or carboxy terminus of the portion, or at both termini, which additional amino acids are not found in the amino acid sequence of the parent CAR. Desirably, the additional amino acids do not interfere with the biological function of the functional portion, e.g., recognize target cells, detect cancer, treat or prevent cancer, etc. More desirably, the additional amino acids enhance the biological activity, as compared to the biological activity of the parent CAR.

Included in the scope of the disclosure are functional variants of the CARs described herein. The term “functional variant” as used herein refers to a CAR, polypeptide, or protein having substantial or significant sequence identity or similarity to a parent CAR, which functional variant retains the biological activity of the CAR of which it is a variant. Functional variants encompass, for example, those variants of the CAR described herein (the parent CAR) that retain the ability to recognize target cells to a similar extent, the same extent, or to a higher extent, as the parent CAR. In reference to the parent CAR, the functional variant can, for instance, be at least about 30%, 50%, 75%, 80%, 90%, 98% or more identical in amino acid sequence to the parent CAR.

A functional variant can, for example, comprise the amino acid sequence of the parent CAR with at least one conservative amino acid substitution. Alternatively or additionally, the functional variants can comprise the amino acid sequence of the parent CAR with at least one non-conservative amino acid substitution. In this case, it is preferable for the non-conservative amino acid substitution to not interfere with or inhibit the biological activity of the functional variant. The non-conservative amino acid substitution may enhance the biological activity of the functional variant, such that the biological activity of the functional variant is increased as compared to the parent CAR.

Amino acid substitutions of the CARs are preferably conservative amino acid substitutions. Conservative amino acid substitutions are known in the art, and include amino acid substitutions in which one amino acid having certain physical and/or chemical properties is exchanged for another amino acid that has the same or similar chemical or physical properties. For instance, the conservative amino acid substitution can be an acidic/negatively charged polar amino acid substituted for another acidic/negatively charged polar amino acid (e.g., Asp or Glu), an amino acid with a nonpolar side chain substituted for another amino acid with a nonpolar side chain (e.g., Ala, Gly, Val, Ile, Leu, Met, Phe, Pro, Trp, Cys, Val, etc.), a basic/positively charged polar amino acid substituted for another basic/positively charged polar amino acid (e.g. Lys, His, Arg, etc.), an uncharged amino acid with a polar side chain substituted for another uncharged amino acid with a polar side chain (e.g., Asn, Gln, Ser, Thr, Tyr, etc.), an amino acid with a beta-branched side-chain substituted for another amino acid with a beta-branched side-chain (e.g., Ile, Thr, and Val.), an amino acid with an aromatic side-chain substituted for another amino acid with an aromatic side chain {e.g., His, Phe, Trp, and Tyr), etc.

Also, amino acids may be added or removed from the sequence based on vector design. For example, the amino acids “SG”, added amino acids due to vector design, may be removed from the CARs as described herein, e.g., removed from the CAR sequences in Table 3, Table 4, or both.

The CAR can consist essentially of the specified amino acid sequence or sequences described herein, such that other components, e.g., other amino acids, do not materially change the biological activity of the functional variant.

The CARs of embodiments of the disclosure (including functional portions and functional variants) can be of any length, i.e., can comprise any number of ammo acids, provided that the CARs (or functional portions or functional variants thereof) retain their biological activity, e.g., the ability to specifically bind to an antigen, detect diseased cells in a mammal, or treat or prevent disease in a mammal, etc. For example, the CAR can be about 50 to about 5000 amino acids long, such as 50, 70, 75, 100, 125, 150, 175, 200, 300, 400, 500, 600, 700, 800, 900, 1000 or more amino acids in length.

The CARs of embodiments of the disclosure (including functional portions and functional variants of the disclosure) can comprise synthetic amino acids in place of one or more naturally-occurring amino acids. Such synthetic amino acids are known in the art, and include, for example, aminocyclohexane carboxylic acid, norleucine, α-amino n-decanoic acid, homoserine, S-acetylaminomethyl-cysteine, trans-3- and trans-4-hydroxyproline, 4-aminophenylalanine, 4-nitrophenylalanine, 4-chlorophenylalanine, 4-carboxyphenylalanine, β-phenylserine β-hydroxyphenylalanine, phenylglycine, α-naphthylalanine, cyclohexylalanine, cyclohexylglycine, indoline-2-carboxylic acid, 1,2,3,4-tetrahydroisoquinoline-3-carboxylic acid, aminomalonic acid, aminomalonic acid monoamide, N′-benzyl-N′-methyl-lysine, N′,N′-dibenzyl-lysine, 6-hydroxylysine, ornithine, α-aminocyclopentane carboxylic acid, α-aminocyclohexane carboxylic acid, α-aminocycloheptane carboxylic acid, α-(2-amino-2-norbornane)-carboxylic acid, α,γ-diaminobutyric acid, α,β-diaminopropionic acid, homophenylalanine, and α-tert-butylglycine.

The CARs of embodiments of the disclosure (including functional portions and functional variants) can be glycosylated, amidated, carboxylated, phosphorylated, esterified, N-acylated, cyclized via, e.g., a disulfide bridge, or converted into an acid addition salt and/or optionally dimerized or polymerized, or conjugated.

The CARs of embodiments of the disclosure (including functional portions and functional variants thereof) can be obtained by methods known in the art. The CARs may be made by any suitable method of making polypeptides or proteins. Suitable methods of de novo synthesizing polypeptides and proteins are described in references, such as Chan et al., Fmoc Solid Phase Peptide Synthesis, Oxford University Press, Oxford, United Kingdom, 2000; Peptide and Protein Drug Analysis, ed. Reid, R., Marcel Dekker, Inc., 2000; Epitope Mapping, ed. Westwood et al, Oxford University Press, Oxford, United Kingdom, 2001; and U.S. Pat. No. 5,449,752. Also, polypeptides and proteins can be recombinantly produced using the nucleic acids described herein using standard recombinant methods. See, for instance, Sambrook et al., Molecular Cloning: A Laboratory Manual, 3rd ed., Cold Spring Harbor Press, Cold Spring Harbor, N.Y. 2001; and Ausubel et al, Current Protocols in Molecular Biology, Greene Publishing Associates and John Wiley & Sons, N Y, 1994. Further, some of the CARs of the disclosure (including functional portions and functional variants thereof) can be isolated and/or purified from a source, such as a plant, a bacterium, an insect, a mammal, e.g., a rat, a human, etc. Methods of isolation and purification are well-known in the art. Alternatively, the CARs described herein (including functional portions and functional variants thereof) can be commercially synthesized by companies. In this respect, the CARs can be synthetic, recombinant, isolated, and/or purified.

Some embodiments of the disclosure further provides an antibody, or antigen binding portion thereof, which specifically binds to an epitope of the CARs of the disclosure. The antibody can be any type of immunoglobulin that is known in the art. For instance, the antibody can be of any isotype, e.g., IgA, IgD, IgE, IgG, IgM, etc. The antibody can be monoclonal or polyclonal. The antibody can be a naturally-occurring antibody, e.g., an antibody isolated and/or purified from a mammal, e.g., mouse, rabbit, goat, horse, chicken, hamster, human, etc. Alternatively, the antibody can be a genetically-engineered antibody, e.g., a humanized antibody or a chimeric antibody. The antibody can be in monomeric or polymeric form. Also, the antibody can have any level of affinity or avidity for the functional portion of the CAR.

Methods of testing antibodies for the ability to bind to any functional portion of the CAR. are known in the art and include any antibody-antigen binding assay, such as, for example, radioimmunoassay (RIA), ELISA, Western blot, immunoprecipitation, and competitive inhibition assays (see, e.g., Janeway et al., infra, U.S. Patent Application Publication No. 2002/0197266 A1, and U.S. Pat. No. 7,338,929).

Suitable methods of making antibodies are known in the art. For instance, standard hybridoma methods are described in, e.g., Köhler and Milstein, Eur. J. Immunol. 5, 511-519 (1976), Harlow and Lane (eds.), Antibodies: A Laboratory Manual, CSH Press (1988), and C. A. Janeway et al. (eds,), Immumobiology, 5th Ed., Garland Publishing, New York, N.Y. (2001). Alternatively, other methods, such as EBV-hybridoma methods (Haskard and Archer, J. Immunol. Methods, 74(2), 361-67 (1984), and Roder et al., Methods Enzymol., 121, 140-67 (1986)), and bacteriophage vector expression systems (see, e.g., Huse et al, Science, 246, 1275-81 (1989)) are known in the art. Further, methods of producing antibodies in non-human animals are described in, e.g., U.S. Pat. Nos. 5,545,806, 5,569,825, and 5,714,352, U.S. Patent Application Publication No. 2002/0197266 A1, and U.S. Pat. No. 7,338,929).

Phage display furthermore can be used to generate an antibody. In this regard, phage libraries encoding antigen-binding variable (V) domains of antibodies can be generated using standard molecular biology and recombinant DNA techniques (see, e.g., Sambrook et al., supra, and Ausubel et al., supra). Phages encoding a variable region with the desired specificity are selected for specific binding to the desired antigen, and a complete or partial antibody is reconstituted comprising the selected variable domain. Nucleic acid sequences encoding the reconstituted antibody are introduced into a suitable cell line, such as a myeloma cell used for hybridoma production, such that antibodies having the characteristics of monoclonal antibodies are secreted by the cell (see, e.g., Janeway et al., supra, Huse et al., supra, and U.S. Pat. No. 6,265,150).

Antibodies can be produced by transgenic mice that are transgenic for specific heavy and light chain immunoglobulin genes. Such methods are known in the art and described in, for example U.S. Pat. Nos. 5,545,806 and 5,569,825, and Janeway et al., supra.

Methods for generating humanized antibodies are well known in the art and are described in detail in, for example, Janeway et al., supra, U.S. Pat. Nos. 5,225,539, 5,585,089 and 5,693,761, European Patent No. 0239400 B1, and United Kingdom Patent No. 2188638. Humanized antibodies can also be generated using the antibody resurfacing technology described in U.S. Pat. No. 5,639,641 and Pedersen et al., J. Mol. Biol., 235, 959-973 (1994).

Some embodiments of the disclosure also provides antigen binding portions of any of the antibodies described herein. The antigen binding portion can be any portion that has at least one antigen binding site, such as Fab, F(ab′)2, dsFv, sFv, diabodies, and triabodies.

A single-chain variable region fragment antibody fragment can be generated using routine recombinant DNA technology techniques (see, e.g., Janeway et al, supra). Similarly, disulfide-stabilized variable region fragments can be prepared by recombinant DNA technology (see, e.g., Reiter et al., Protein Engineering, 7, 697-704 (1994)). Antibody fragments of the disclosure, however, are not limited to these exemplary types of antibody fragments.

Also, the antibody, or antigen binding portion thereof, can be modified to comprise a detectable label, such as, for instance, a radioisotope, a fluorophore (e.g., fluorescein isothiocyanate (F1TC), phycoerythrin (PE)), an enzyme (e.g., alkaline phosphatase, horseradish peroxidase), and element particles (e.g., gold particles).

Further provided by some embodiments of the disclosure is a nucleic acid molecule comprising a nucleotide sequence encoding any of the CARs described herein (including functional portions and functional variants thereof). The nucleic acids of the disclosure may comprise a nucleotide sequence encoding any of the leader sequences, antigen binding domains, transmembrane domains, and/or intracellular T cell signaling domains described herein.

In some embodiments, the nucleotide sequence may be codon-optimized. Without being bound to a particular theory, it is believed that codon optimization of the nucleotide sequence increases the translation efficiency of the mRNA transcripts, Codon optimization of the nucleotide sequence may involve substituting a native codon for another codon that encodes the same amino acid, but can be translated by tRNA that is more readily available within a cell, thus increasing translation efficiency. Optimization of the nucleotide sequence may also reduce secondary mRNA structures that would interfere with translation, thus increasing translation efficiency.

In some embodiments of the disclosure, the nucleic acid may comprise a codon-optimized nucleotide sequence that encodes the antigen binding domain of the CAR. In another embodiment of the disclosure, the nucleic acid may comprise a codon-optimized nucleotide sequence that encodes any of the CARs described herein (including functional portions and functional variants thereof).

“Nucleic acid” as used herein includes “polynucleotide,” “oligonucleotide,” and “nucleic acid molecule,” and generally means a polymer of DNA or RNA, which can be single-stranded or double-stranded, synthesized or obtained (e.g., isolated and/or purified) from natural sources, which can contain natural, non-natural or altered nucleotides, and which can contain a natural, non-natural or altered internucleotide linkage, such as a phosphoroamidate linkage or a phosphorothioate linkage, instead of the phosphodiester found between the nucleotides of an unmodified oligonucleotide, in some embodiments, the nucleic acid does not comprise any insertions, deletions, inversions, and/or substitutions. However, it may be suitable in some instances, as discussed herein, for the nucleic acid to comprise one or more insertions, deletions, inversions, and/or substitutions.

The nucleic acids of some embodiments of the disclosure may be recombinant. As used herein, the term “recombinant” refers to (i) molecules that are constructed outside living cells by joining natural or synthetic nucleic acid segments to nucleic acid molecules that can replicate in a living cell, or (ii) molecules that result from the replication of those described in (i) above. For purposes herein, the replication can be in vitro replication or in vivo replication.

A recombinant nucleic acid may be one that has a sequence that is not naturally occurring or has a sequence that is made by an artificial combination of two otherwise separated segments of sequence. This artificial combination is often accomplished by chemical synthesis or, more commonly, by the artificial manipulation of isolated segments of nucleic acids, e.g., by genetic engineering techniques, such as those described in Sambrook et al., supra. The nucleic acids can be constructed based on chemical synthesis and/or enzymatic ligation reactions using procedures known in the art. See, for example, Sambrook et al., supra, and Ausubel et al. supra. For example, a nucleic acid can be chemically synthesized using naturally occurring nucleotides or variously modified nucleotides designed to increase the biological stability of the molecules or to increase the physical stability of the duplex formed upon hybridization (e.g., phosphorothioate derivatives and acridine substituted nucleotides). Examples of modified nucleotides that can be used to generate the nucleic acids include, but are not limited to, 5-flurouracil, 5-bromouracil, 5-chlorouracil, 5-iodouracil, hypoxanthine, xanthine, 4-acetylcytosine, 5-(carboxyhydroxymethyl) uracil, 5-carboxymethylaminomethyl-2-thiouridine, 5-carboxymethylaminomethyluracil, dihydrouracil, beta-D-galactosylqueosine, inosine, N6-isopenenyladenine, 1-methylguanine, 1-methylinosine, 2,2-dimethylguanine, 2-methyladenine, 2-methylguanine, 3-methylcytosine, 5-methylcytosine, N6-substituted adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, 3-(3-amino-3-N-2-carboxypropyl) uracil, and 2,6-diaminopurine. Alternatively, one or more of the nucleic acids of the disclosure can be purchased from companies, such as integrated DNA Technologies (Coralville, Iowa, USA).

The nucleic acid can comprise any isolated or purified nucleotide sequence which encodes any of the CARs or functional portions or functional variants thereof. Alternatively, the nucleotide sequence can comprise a nucleotide sequence which is degenerate to any of the sequences or a combination of degenerate sequences.

Some embodiments of the disclosure also provides an isolated or purified nucleic acid comprising a nucleotide sequence which is complementary to the nucleotide sequence of any of the nucleic acids described herein or a nucleotide sequence which hybridizes under stringent conditions to the nucleotide sequence of any of the nucleic acids described herein.

The nucleotide sequence which hybridizes under stringent conditions may hybridize under high stringency conditions. By “high stringency conditions” is meant that the nucleotide sequence specifically hybridizes to a target sequence (the nucleotide sequence of any of the nucleic acids described herein) in an amount that is detectably stronger than non-specific hybridization. High stringency conditions include conditions which would distinguish a polynucleotide with an exact complementary sequence, or one containing only a few scattered mismatches from a random sequence that happened to have a few small regions (e.g., 3-10 bases) that matched the nucleotide sequence. Such small regions of complementarity are more easily melted than a full-length complement of 14-17 or more bases, and high stringency hybridization makes them easily distinguishable. Relatively high stringency conditions would include, for example, low salt and/or high temperature conditions, such as provided by about 0.02-0.1 M NaCl or the equivalent, at temperatures of about 50-70° C. Such high stringency conditions tolerate little, if any, mismatch between the nucleotide sequence and the template or target strand, and are particularly suitable for detecting expression of any of the CARs. It is generally appreciated that conditions can be rendered more stringent by the addition of increasing amounts of formamide.

The disclosure also provides a nucleic acid comprising a nucleotide sequence that is at least about 70% or more, e.g., about 80%, about 90%, about 91%, about 92%, about 93%, about 94%, about 95%, about 96%, about 97%, about 98%, or about 99% identical to any of the nucleic acids described herein.

In some embodiments, the nucleic acids of the disclosure can be incorporated into a recombinant expression vector. In this regard, some embodiments of the disclosure provides recombinant expression vectors comprising any of the nucleic acids of the disclosure, For purposes herein, the term “recombinant expression vector” means a genetically-modified oligonucleotide or polynucleotide construct that permits the expression of an mRNA, protein, polypeptide, or peptide by a host cell, when the construct comprises a nucleotide sequence encoding the mRNA, protein, polypeptide, or peptide, and the vector is contacted with the cell under conditions sufficient to have the mRNA, protein, polypeptide, or peptide expressed within the cell. The vectors of the disclosure are not naturally-occurring as a whole. However, parts of the vectors can be naturally-occurring. The recombinant expression vectors can comprise any type of nucleotides, including, but not limited to DNA and RNA, which can be single-stranded or double-stranded, synthesized or obtained in part from natural sources, and which can contain natural, non-natural or altered nucleotides. The recombinant expression vectors can comprise naturally-occurring or non-naturally-occurring internucleotide linkages, or both types of linkages. Preferably, the non-naturally occurring, or altered nucleotides or internucleotide linkages do not hinder the transcription or replication of the vector.

In some embodiments, the recombinant expression vector of the disclosure can be any suitable recombinant expression vector, and can be used to transform or transfect any suitable host cell. Suitable vectors include those designed for propagation and expansion or for expression or both, such as plasmids and viruses. The vector can be selected from the group consisting of the pUC series (Fermentas Life Sciences, Glen Burnie, Md.), the pBluescript series (Stratagene, LaJolla, Calif.), the pET series (Novagen, Madison, Wis.), the pGEX series (Pharmacia Biotech, Uppsala, Sweden), and the pEX series (Clontech, Palo Alto, Calif.). Bacteriophage vectors, such as λGT10, λGT11 I, λZapII (Stratagene), λEMBL4, and λNM1149, also can be used. Examples of plant expression vectors include pBI01, pBI101.2, pBI101.3, pBI121 and pBIN19 (Clontech). Examples of animal expression vectors include pEUK-Cl, pMAM, and pMAMneo (Clontech). The recombinant expression vector may be a viral vector, e.g., a retroviral vector or a lentiviral vector.

A number of transfection techniques are generally known in the art (see, e.g., Graham et al., Virology, 52: 456-467 (1973); Sambrook et al., supra; Davis et al., Basic Methods in Molecular Biology, Elsevier (1986); and Chu et al., Gene, 13: 97 (1981). Transfection methods include calcium phosphate co-precipitation (see, e.g., Graham et al., supra), direct micro injection into cultured cells (see, e.g., Capecchi, Cell, 22: 479-488 (1980)), electroporation (see, e.g., Shigekawa et al., BioTechniqaes, 6: 742-751 (1988)), liposome mediated gene transfer (see, e.g., Mannino et al., BioTechniques, 6: 682-690 (1988)), lipid mediated transduction (see, e.g., Felgner et al., Proc. Natl. Acad. Sci. USA, 84: 7413-7417 (1987)), and nucleic acid delivery using high velocity microprojectiles (see, e.g., Klein et al., Nature, 327: 70-73 (1987)).

In some embodiments, the recombinant expression vectors of the disclosure can be prepared using standard recombinant DNA techniques described in, for example, Sambrook et al., supra, and Ausubel et al., supra. Constructs of expression vectors, which are circular or linear, can be prepared to contain a replication system functional in a prokaryotic or eukaryotic host cell, Replication systems can be derived, e.g., from ColEl, 2μ plasmid, λ, SV40, bovine papilloma virus, and the like.

The recombinant expression vector may comprise regulatory sequences, such as transcription and translation initiation and termination codons, which are specific to the type of host cell (e.g., bacterium, fungus, plant, or animal) into which the vector is to be introduced, as appropriate, and taking into consideration whether the vector is DNA- or RNA-based. The recombinant expression vector may comprise restriction sites to facilitate cloning.

The recombinant expression vector can include one or more marker genes, which allow for selection of transformed or transfected host cells. Marker genes include biocide resistance, e.g., resistance to antibiotics, heavy metals, etc., complementation in an auxotrophic host to provide prototrophy, and the like. Suitable marker genes for the expression vectors include, for instance, neomycin/G418 resistance genes, hygromycin resistance genes, histidinol resistance genes, tetracycline resistance genes, and ampicillin resistance genes.

The recombinant expression vector can comprise a native or nonnative promoter operably linked to the nucleotide sequence encoding the CAR (including functional portions and functional variants thereof), or to the nucleotide sequence which is complementary to or which hybridizes to the nucleotide sequence encoding the CAR. The selection of promoters, e.g., strong, weak, inducible, tissue-specific and developmental-specific, is within the ordinary skill of the artisan. Similarly, the combining of a nucleotide sequence with a promoter is also within the skill of the artisan. The promoter can be a non-viral promoter or a viral promoter, e.g., a cytomegalovirus (CMV) promoter, an SV40 promoter, an RSV promoter, or a promoter found in the long-terminal repeat of the murine stem cell virus.

The recombinant expression vectors can be designed for either transient expression, for stable expression, or for both. Also, the recombinant expression vectors can be made for constitutive expression or for inducible expression.

Further, the recombinant expression vectors can be made to include a suicide gene. As used herein, the term “‘suicide gene” refers to a gene that causes the cell expressing the suicide gene to die. The suicide gene can be a gene that confers sensitivity to an agent, e.g., a drug, upon the cell in which the gene is expressed, and causes the cell to die when the cell is contacted with or exposed to the agent. Suicide genes are known in the art (see, for example, Suicide Gene Therapy: Methods and Reviews, Springer, Caroline J. (Cancer Research UK Centre for Cancer Therapeutics at the Institute of Cancer Research, Sutton, Surrey, UK), Humana Press, 2004) and include, for example, the Herpes Simplex Virus (HSV) thymidine kinase (TK) gene, cytosine deaminase, purine nucleoside phosphorylase, and nitroreductase.

Included in the scope of the disclosure are conjugates, e.g., bioconjugates, comprising any of the CARs (including any of the functional portions or variants thereof), nucleic acids, recombinant expression vectors, host cells, populations of host cells, or antibodies, or antigen binding portions thereof. Conjugates, as well as methods of synthesizing conjugates in general, are known in the art (See, for instance, Hudecz, F., Methods Mol. Biol., 298: 209-223 (2005) and Kirin et al., Inorg Chem. 44(15): 5405-5415 (2005)). CARs of the disclosure may be conjugated to, e.g., toxins that are toxic to cancer cells.

Some embodiments of the disclosure further provides a host cell comprising any of the recombinant expression vectors described herein. As used herein, the term “host cell” refers to any type of cell that can contain the recombinant expression vector. The host cell can be a eukaryotic cell, e.g., plant, animal, fungi, or algae, or can be a prokaryotic cell, e.g., bacteria or protozoa. The host cell can be a cultured cell or a primary cell, i.e., isolated directly from an organism, e.g., a human. The host cell can be an adherent cell or a suspended cell, i.e., a cell that grows in suspension. Suitable host cells are known in the art and include, for instance, DK5α E. coli cells, Chinese hamster ovaria cells, monkey VERO cells, COS cells, HEK293 cells, and the like. For purposes of amplifying or replicating the recombinant expression vector, the host cell may be a prokaryotic cell, e.g., a DH5α cell. For the purposes of producing a recombinant CAR, the host cell may be a mammalia cell. The host cell may be a huma cell. While the host cell can be of any cell type, can originate from any type of tissue, and can be of any developmental stage, the host cell may be a peripheral blood lymphocyte (PBL) or a peripheral blood mononuclear cell (PBMC). The host cell may be a T cell.

For purposes herein, the T cell can be any T cell, such as a cultured T cell, e.g., a primary T cell, or a T cell from a cultured T cell line, e.g., Jurkat, SupT1, etc., or a T cell obtained from a mammal. If obtained from a mammal, the T cell can be obtained from numerous sources, including but not limited to blood, bone marrow, lymph node, the thymus, or other tissues or fluids. T cells can also be enriched for or purified. The T cell may be a human T cell. The T cell may be a T cell isolated from a human. The T cell can be any type of cell and can be of any developmental stage, including but not limited to, CD4+/CD8+ double positive T cells, CD4+ helper T cells, e.g., Th1 and Th2 cells, CD8+ T cells (e.g., cytotoxic T cells), tumor infiltrating cells, memory T cells, naive T cells, and the like. The T cell may be a CD8+ T cell or a CD4+ T cell. In some embodiments, CARs specific for different antigens may be expressed on different cells within a population of cells. These cells may be the same or different type of cell.

In some embodiments, the CARs as described herein can be used in suitable non-T cells. Such cells are those with an immune-effector function, such as, for example, NK cells, and T-like cells generated from pluripotent stem cells.

Also provided by some embodiments of the disclosure is a population of cells comprising at least one host cell described herein, The population of cells can be a heterogeneous population comprising the host cell comprising any of the recombinant expression vectors described, in addition to at least one other cell, e.g., a host cell (e.g., a T cell), which does not comprise any of the recombinant expression vectors, or a cell other than a T cell, e.g., a B cell, a macrophage, a neutrophil, an erythrocyte, a hepatocyte, an endothelial cell, an epithelial cell a muscle cell, a brain cell, etc. Alternatively, the population of cells can be a substantially homogeneous population, in which the population comprises mainly host cells (e.g., consisting essentially of) comprising the recombinant expression vector. The population also can be a clonal population of cells, in which all cells of the population are clones of a single host cell comprising a recombinant expression vector, such that all cells of the population comprise the recombinant expression vector. In one embodiment of the disclosure, the population of cells is a clonal population comprising host cells comprising a recombinant expression vector as described herein. Alternatively, the population of cells can be a heterogeneous population comprising a first host cell comprising a recombinant expression vector comprising a nucleic acid molecule encoding a first CAR comprising an antigen binding domain with specificity for FLT3 and an intracellular T cell signaling domain, and a second host cell comprising a recombinant expression vector comprising a nucleic acid molecule encoding encoding a second CAR comprising an antigen binding domain with specificity for a second antigen and an intracellular T cell signaling domain

CARs (including functional portions and variants thereof), nucleic acids, recombinant expression vectors, host cells (including populations thereof), and antibodies (including antigen binding portions thereof), all of which are collectively referred to as “CAR materials” hereinafter, can be isolated and/or purified. The term “isolated” as used herein means having been removed from its natural environment. The term “purified” or “isolated” does not require absolute purity or isolation; rather, it is intended as a relative term. Thus, for example, a purified (or isolated) host cell preparation is one in which the host cell is purer tha cells in their natural environment within the body. Such host cells may be produced, for example, by standard purification techniques. In some embodiments, a preparation of a host cell is purified such that the host cell represents at least about 50%, for example at least about 70%, of the total cell content of the preparation. For example, the purity can be at least about 50%, can be greater than about 60%, about 70% or about 80%, or can be about 100%.

The CAR materials can be formulated into a composition, such as a pharmaceutical composition. In this regard, some embodiments of the disclosure provides a pharmaceutical composition comprising any of the CARs, functional portions, functional variants, nucleic acids, expression vectors, host cells (including populations thereof), and antibodies (including antigen binding portions thereof), and a pharmaceutically acceptable carrier. The pharmaceutical compositions containing any of the CAR materials can comprise more than one CAR material, e.g., a CAR and a nucleic acid, or two or more different CARs. Alternatively, the pharmaceutical composition can comprise an CAR material in combination with other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, vincristine, etc. in a preferred embodiment, the pharmaceutical composition comprises the host cell or populations thereof.

The CAR materials can be provided in the form of a salt, e.g., a pharmaceutically acceptable salt. Suitable pharmaceutically acceptable acid addition salts include those derived from mineral acids, such as hydrochloric, hydrobromic, phosphoric, metaphosphoric, nitric, and sulphuric acids, and organic acids, such as tartaric, acetic, citric, malic, lactic, fumaric, benzoic, glycolic, gluconic, succinic, and arylsulphonic acids, for example, p-toluenesulphonic acid.

With respect to pharmaceutical compositions, the pharmaceutically acceptable carrier can be any of those conventionally used and is limited only by chemico-physical considerations, such as solubility and lack of reactivity with the active agent(s), and by the route of administration. The pharmaceutically acceptable carriers described herein, for example, vehicles, adjuvants, excipients, and diluents, are well-known to those skilled in the art and are readily available to the public. It is preferred that the pharmaceutically acceptable carrier be one which is chemically inert to the active agent(s) and one which has no detrimental side effects or toxicity under the conditions of use.

The choice of carrier will be determined in part by the particular CAR material, as well as by the particular method used to administer the CAR material. Accordingly, there are a variety of suitable formulations of the pharmaceutical composition of the disclosure. Preservatives may be used. Suitable preservatives may include, for example, methylparaben, propylparaben, sodium benzoate, and benzalkonium chloride. A mixture of two or more preservatives optionally may be used. The preservative or mixtures thereof are typically present in an amount of about 0.0001% to about 2% by weight of the total composition.

Suitable buffering agents may include, for example, citric acid, sodium citrate, phosphoric acid, potassium phosphate, and various other acids and salts. A mixture of two or more buffering agents optionally may be used. The buffering agent or mixtures thereof are typically present in an amount of about 0.001% to about 4% by weight of the total composition.

The concentration of CAR material in the pharmaceutical formulations can vary, e.g., from less than about 1%, at or at least about 10%, to as much as about 20% to about 50% or more by weight, and can be selected primarily by fluid volumes, and viscosities, in accordance with the particular mode of administration selected.

Methods for preparing administrable (e.g., parenterally administrable) compositions are known or apparent to those skilled in the art and are described in more detail in, for example. Remington: The Science and Practice of Pharmacy, Lippincott Williams & Wilkins; 21st ed. (May 1, 2005).

The following formulations for oral, aerosol, parenteral (e.g., subcutaneous, intravenous, intraarterial, intramuscular, intradermal, interperitoneal, and intrathecal), and topical administration are merely exemplary and are in no way limiting. More than one route can be used to administer the CAR materials, and in certain instances, a particular route can provide a more immediate and more effective response than another route.

Formulations suitable for oral administration can comprise or consist of (a) liquid solutions, such as an effective amount of the CAR material dissolved in diluents, such as water, saline, or orange juice; (b) capsules, sachets, tablets, lozenges, and troches, each containing a predetermined amount of the active ingredient, as solids or granules; (c) powders; (d) suspensions in an appropriate liquid; and (e) suitable emulsions. Liquid formulations may include diluents, such as water and alcohols, for example, ethanol, benzyl alcohol and the polyethylene alcohols, either with or without the addition of a pharmaceutically acceptable surfactant. Capsule forms can be of the ordinary hard or softshelled gelatin type containing, for example, surfactants, lubricants, and inert fillers, such as lactose, sucrose, calcium phosphate, and corn starch. Tablet forms can include one or more of lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and other pharmacologically compatible excipients. Lozenge forms can comprise the CAR material in a flavor, usually sucrose and acacia or tragacanth, as well as pastilles comprising the CAR material in an inert base, such as gelatin and glycerin, or sucrose and acacia, emulsions, gels, and the like containing, in addition to, such excipients as are known in the art.

Formulations suitable for parenteral administration include aqueous and nonaqueous isotonic sterile injection solutions, winch can contain antioxidants, buffers, bacteriostats, and solutes that render the formulation isotonic with the blood of the intended recipient, and aqueous and nonaqueous sterile suspensions that can include suspending agents, solubilizers, thickening agents, stabilizers, and preservatives. The CAR material can be administered in a physiologically acceptable diluent in a pharmaceutical carrier, such as a sterile liquid or mixture of liquids, including water, saline, aqueous dextrose and related sugar solutions, an alcohol, such as ethanol or hexadecyl alcohol, a glycol, such as propylene glycol or polyethylene glycol, dimethylsulfoxide, glycerol, ketals such as 2,2-dimethyl-1,3-dioxolane-4-methanol, ethers, poly(ethyleneglycol) 400, oils, fatty acids, fatty acid esters or glycerides, or acetylated fatty acid glycerides with or without the addition of a pharmaceutically acceptable surfactant such as a soap or a detergent, suspending agent, such as pectin, carbomers, methylcellulose, hydroxypropylmethylcellulose, or carboxymethylcellulose, or emulsifying agents and other pharmaceutical adjuvants.

Oils, which can be used in parenteral formulations, include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters.

Suitable soaps for use in parenteral formulations include fatty alkali metal, ammonium, and triethanolamme salts, and suitable detergents include (a) cationic detergents such as, for example, dimethyl dialkyl ammonium halides. and alkyl pyridinium halides, (b) anionic detergents such as, for example, alkyl, aryl, and olefin sulfonates, alky, olefin, ether, and monoglyceride sulfates, and sulfosuccinates, (c) nonionic detergents such as, for example, fatty amine oxides, fatty acid alkanolamides, and polyoxyethylenepolypropylene copolymers, (d) amphoteric detergents such as, for example, alkyl-β-aminopropionates, and 2-alkyi-imidazoline quaternary ammonium salts, and (e) mixtures thereof.

The parenteral formulations will typically contain, for example, from about 0.5% to about 25% by weight of the CAR material in solution. Preservatives and buffers may be used. In order to minimize or eliminate irritation at the site of injection, such compositions may contain one or more nonionic surfactants having, for example, a hydrophile-lipophile balance (HLB) of from about 12 to about 17. The quantity of surfactant in such formulations will typically range, for example, from about 5% to about 15% by weight. Suitable surfactants include polyethylene glycol sorbitan fatty acid esters, such as sorbitan monooleate and the high molecular weight adducts of ethylene oxide with a hydrophobic base, formed by the condensation of propylene oxide with propylene glycol. The parenteral formulations can be presented in unit-dose or multi-dose sealed containers, such as ampoules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described.

Injectable formulations are in accordance with some embodiments of the disclosure. The requirements for effective pharmaceutical carriers for injectable compositions are well-known to those of ordinary skill in the art (see, e.g., Pharmaceutics and Pharmacy Practice, J.B. Lippincott Company, Philadelphia, Pa., Banker and Chalmers, eds., pages 238-250 (1982), and ASHP Handbook on Injectable Drugs, Toissel, 4th ed, pages 622-630 (1986)).

Topical formulations, including those that are useful for transdermal drug release, are well known to those of skill in the art and are suitable in the context of embodiments of the disclosure for application to skin. The CAR material, alone or in combination with other suitable components, can be made into aerosol formulations to be administered via inhalation. These aerosol formulations can be placed into pressurized acceptable propellants, such as dichlorodifluoromethane, propane, nitrogen, and the like. They also may be formulated as pharmaceuticals for non-pressured preparations, such as in a nebulizer or an atomizer. Such spray formulations also may be used to spray mucosa.

An “effective amount” or “an amount effective to treat” refers to a dose that is adequate to prevent or treat cancer in an individual. Amounts effective for a therapeutic or prophylactic use will depend on, for example, the stage and severity of the disease or disorder being treated, the age, weight, and general state of health of the patient, and the judgment of the prescribing physician. The size of the dose will also be determined by the active selected, method of administration, timing and frequency of administration, the existence, nature, and extent of any adverse side-effects that might accompany the administration of a particular active, and the desired physiological effect. It will be appreciated by one of skill in the art that various diseases or disorders could require prolonged treatment involving multiple administrations, perhaps using the CAR materials in each or various rounds of administration. By way of example and not intending to limit the disclosure, the dose of the CAR material can be about 0.001 to about 1000 mg/kg body weight of the subject being treated/day, from about 0.01 to about 10 mg/kg body weight/day, about 0.01 mg to about 1 mg/kg body weight/day. In some embodiments some embodiments of the disclosure, the dose may be from about 1×104 to about 1×108 cells expressing the CAR material per kg body weight. When the CAR material is a host cell, an exemplary dose of host cells may be a minimum of one million cells (1 mg cells/dose), When the CAR material is a nucleic acid packaged in a virus, an exemplary dose of virus may be 1 ng/dose.

For purposes of the disclosure, the amount or dose of the CAR material administered should be sufficient to effect a therapeutic or prophylactic response in the subject or animal over a reasonable time frame. For example, the dose of the CAR material should be sufficient to bind to antigen, or detect, treat or prevent disease in a period of from about 2 hours or longer, e.g., about 12 to about 24 or more hours, from the time of administration. In some embodiments, the time period could be even longer. The dose will be determined by the efficacy of the particular CAR material and the condition of the animal (e.g., human), as well as the body weight of the animal (e.g., human) to be treated.

For the purposes of the disclosure, an assay, which comprises, for example, comparing the extent to which target cells are lysed and/or IFN-γ is secreted by T cells expressing the CAR upon administration of a given dose of such T cells to a mammal, among a set of mammals of which is each given a different dose of the T cells, could be used to determine a starting dose to be administered to a mammal. The extent to which target cells are lysed and/or IFN-γ is secreted upon administration of a certain dose can be assayed by methods known in the art.

In addition to the aforedescribed pharmaceutical compositions, the CAR materials can be formulated as inclusion complexes, such as cyclodextrin inclusion complexes, or liposomes. Liposomes can serve to target the CAR materials to a particular tissue. Liposomes also can be used to increase the half-life of the CAR materials. Many methods are available for preparing liposomes, as described in, for example, Szoka et al., Ann. Rev. Biophys. Bioeng., 9, 467 (1980) and U.S. Pat. Nos. 4,235,871, 4,501,728, 4,837,028, and 5,019,369.

The delivery systems useful in the context of embodiments of the disclosure may include time-released, delayed release, and sustained release delivery systems such that the delivery of the composition occurs prior to, and with sufficient time to cause, sensitization of the site to be treated. The composition can be used in conjunction with other therapeutic agents or therapies. Such systems can avoid repeated administrations of the composition, thereby increasing convenience to the subject and the physician, and may be particularly suitable for certain composition embodiments of the disclosure.

Contemplated types of release delivery systems include polymer base systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are lipids including sterols such as cholesterol, cholesterol esters, and fatty acids or neutral fats such as mono-di- and tri-glycerides; hydrogel release systems; sylastic systems; peptide based systems: wax coatings; compressed tablets using conventional binders and excipients; partially fused implants; and the like. Specific examples include, but are not limited to: (a) erosional systems in which the active composition is contained in a form within a matrix such as those described in U.S. Pat. Nos. 4,452,775, 4,667,014, 4,748,034, and 5,239,660 and (b) diffusional systems in which an active component permeates at a controlled rate from a polymer such as described in U.S. Pat. Nos. 3,832,253 and 3,854,480. In addition, pump-based hardware delivery systems can be used, some of which are adapted for implantation.

One of ordinary skill in the art will readily appreciate that the CAR materials of the disclosure can be modified in any number of ways, such that the therapeutic or prophylactic efficacy of the CAR materials is increased through the modification. For instance, the CAR materials can be conjugated either directly or indirectly through a linking moiety to a targeting moiety. The practice of conjugating compounds, e.g., CAR materials, to targeting moieties is known in the art. See, for instance, Wadwa et al., J, Drug Targeting 3:111 (1995) and U.S. Pat. No. 5,087,616.

The CAR materials can be modified into a depot form, such that the manner in which the CAR materials is released into the body to which it is administered is controlled with respect to time and location within the body (see, for example, U.S. Pat. No. 4,450,150). Depot forms of CAR materials can be, for example, an implantable composition comprising the CAR materials and a porous or non-porous material, such as a polymer, wherein the CAR materials are encapsulated by or diffused throughout the material and/or degradation of the non-porous material. The depot is then implanted into the desired location within the body and the CAR materials are released from the implant at a predetermined rate.

When the CAR materials are administered with one or more additional therapeutic agents, one or more additional therapeutic agents can be coadministered to the mammal. By “coadministering” is meant administering one or more additional therapeutic agents and the CAR materials sufficiently close in time such that the CAR materials can enhance the effect of one or more additional therapeutic agents, or vice versa. In this regard, the CAR materials can be administered first and the one or more additional therapeutic agents can be administered second, or vice versa. Alternatively, the CAR materials and the one or more additional therapeutic agents can be administered simultaneously.

An exemplary therapeutic agent that can be co-administered with the CAR materials is a T cell active cytokine, such as IL-2. It is believed that IL-2 enhances the therapeutic effect of the CAR materials. Without being bound by a particular theory or mechanism, it is believed that IL-2 enhances therapy by enhancing the in vivo expansion of the numbers and/or effector function of cells expressing the CARs, Other exemplary cytokines include IL-7 and IL-15. For purposes of the methods, wherein host cells or populations of cells are administered to the mammal, the cells can be cells that are allogeneic or autologous to the mammal.

It is contemplated that the CAR materials can be used in methods of treating or preventing a disease in a mammal. Without being bound to a particular theory or mechanism, the CARs have biological activity, e.g., ability to recognize an antigen, e.g., FLT3, such that the CAR when expressed by a cell is able to mediate an immune response against the cell expressing the antigen, e.g., FLT3, for which the CAR is specific. In this regard, some embodiments of the disclosure provides a method of treating or preventing cancer in a mammal, comprising administering to the mammal the CARs, the nucleic acids, the recombinant expression vectors, the host cells, the population of cells, the antibodies and/or the antigen binding portions thereof, and/or the pharmaceutical compositions of the disclosure in an amount effective to treat or prevent cancer in the mammal.

Some embodiments of the disclosure further comprises lymphodepleting the mammal prior to administering the CAR materials. Examples of lymphodepletion include, but may not be limited to, nonmyeloablative lymphodepleting chemotherapy, myeloablative lymphodepleting chemotherapy, total body irradiation, etc.

For the purposes of the methods, wherein host cells or populations of cells are administered, the cells can be cells that are allogeneic or autologous to the mammal. Preferably, the cells are autologous to the mammal.

The mammal referred to herein can be any mammal. As used herein, the term “mammal” refers to any mammal, including, but not limited to, mammals of the order Rodentia, such as mice and hamsters, and mammals of the order Logomorpha, such as rabbits. The mammals may be from the order Carnivora, including Felines (cats) and Canines (dogs). The mammals may be from the order Artiodactyla, including Bovines (cows) and Swines (pigs) or of the order Perssodactyla, including Equines (horses). The mammals may be of the order Primates, Ceboids, or Simoids (monkeys) or of the order Anthropoids (humans and apes). Preferably, the mammal is a human.

With respect to the methods, the cancer can be any cancer, including any of acute lymphocytic cancer, acute myeloid leukemia (AML), treatment resistant acute myeloid leukemia, alveolar rhabdomyosarcoma, bladder cancer (e.g., bladder carcinoma), bone cancer, brain cancer (e.g., medulloblastoma), breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vulva, chronic lymphocytic leukemia, chronic myeloid cancer, colon cancer, esophageal cancer, cervical cancer, fibrosarcoma, gastrointestinal carcinoid tumor, head and neck cancer (e.g., head and neck squamous cell carcinoma), Hodgkin lymphoma, hypopharynx cancer, kidney cancer, larynx cancer, leukemia, liquid tumors, liver cancer, lung cancer (e.g., non-small cell lung carcinoma and lung adenocarcinoma), lymphoma, mesothelioma, mastocytoma, melanoma, multiple myeloma, nasopharynx cancer, non-Hodgkin lymphoma, B-chronic lymphocytic leukemia, hairy cell leukemia, acute lymphocytic leukemia (ALL), treatment resistant acute lymphocytic leukemia, and Burkitt's lymphoma, ovarian cancer, pancreatic cancer, peritoneum, omentum, and mesentery cancer, pharynx cancer, prostate cancer, rectal cancer, renal cancer, skin cancer, small intestine cancer, soft tissue cancer, solid tumors, synovial sarcoma, gastric cancer, testicular cancer, thyroid cancer, and ureter cancer. Preferably, the cancer is characterized by the expression of FLT3.

The terms “treat,” and “prevent” as well as words stemming therefrom, as used herein, do not necessarily imply 100% or complete treatment or prevention. Rather, there are varying degrees of treatment or prevention of which one of ordinary skill in the art recognizes as having a potential benefit or therapeutic effect, in this respect, the methods can provide any amount or any level of treatment or prevention of cancer in a mammal. Furthermore, the treatment or prevention provided by the method can include treatment or prevention of one or more conditions or symptoms of the disease, e.g., cancer, being treated or prevented. Also, for purposes herein, “prevention” can encompass delaying the onset of the disease, or a symptom or condition thereof.

Another embodiment of the disclosure provides a method of detecting the presence of cancer in a mammal, comprising: (a) contacting a sample comprising one or more cells from the mammal with the CARs, the nucleic acids, the recombinant expression, vectors, the host cells, the population of cells, the antibodies, and/or the antigen binding portions thereof, or the pharmaceutical compositions of the disclosure, thereby forming a complex, (b) and detecting the complex, wherein detection of the complex is indicative of the presence of cancer in the mammal.

Another embodiment of the disclosure includes a method of determining whether a subject with a proliferative disorder is a candidate for treatment with a chimeric antigen receptor comprising an antigen binding domain specific for FLT3, the method comprising measuring FLT3 expression levels in a biological sample from the subject; and determining if the FLT3 expression levels of the biological sample are increased compared to a sample from a control subject without the proliferative disorder.

The sample may be obtained by any suitable method, e.g., biopsy or necropsy. A biopsy is the removal of tissue and/or cells from an individual. Such removal may be to collect tissue and/or cells from the individual in order to perform experimentation on the removed tissue and/or cells. This experimentation may include experiments to determine if the individual has and/or is suffering from a certain condition or disease-state. The condition or disease may be, e.g., cancer.

With respect to some embodiments of the method of detecting the presence of a proliferative disorder, e.g., cancer, in a mammal, the sample comprising cells of the mammal can be a sample comprising whole cells, lysates thereof, or a fraction of the whole cell lysates, e.g., a nuclear or cytoplasmic fraction, a whole protein fraction, or a nucleic acid fraction. If the sample comprises whole cells, the cells can be any cells of the mammal, e.g., the cells of any organ or tissue, including blood cells or endothelial cells.

The contacting can take place in vitro or in vivo with respect to the mammal. Preferably, the contacting is in vitro.

Also, detection of the complex can occur through any number of ways known in the art. For instance, the CARs, polypeptides, proteins, nucleic acids, recombinant expression vectors, host cells, populations of cells, or antibodies, or antigen binding portions thereof, described herein, can be labeled with detectable label such as, for instance, a radioisotope, a fluorophore (e.g., fluorescein isothiocyanate (FITC), phycoerythrin (PE)), an enzyme (e.g., alkaline phosphatase, horseradish peroxidase), and element particles (e.g., gold particles).

Methods of testing a CAR for the ability to recognize target cells and for antigen specificity are known in the art. For instance, Clay et al., J. Immunol., 163: 507-513 (1999), teaches methods of measuring the release of cytokines (e.g., interferon-γ, granulocyte/monocyte colony stimulating factor (GM-CSF), tumor necrosis factor α (TNF-α) or interleukin 2 (IL-2)). In addition, CAR function can be evaluated by measurement of cellular cytotoxicity, as described in Zhao et al., J. Immunol., 174: 4415-4423 (2005).

Another embodiment of the disclosure provides the use of the CARs, nucleic acids, recombinant expression vectors, host cells, populations of cells, antibodies, or antigen binding portions thereof, and/or pharmaceutical compositions of the disclosure, for the treatment or prevention of a proliferative disorder, e.g., cancer, in a mammal. The cancer may be any of the cancers described herein. Preferably, the cancer is pre-B cell precursor acute lymphoblastic leukemia or acute myeloid leukemia.

It shall be noted that the preceding are merely examples of embodiments. Other exemplary embodiments are apparent from the entirety of the description herein. It will also be understood by one of ordinary skill in the art that each of these embodiments may be used in various combinations with the other embodiments provided herein.

### EXAMPLES

The following examples further illustrate the disclosure but, of course, should not be construed as in any way limiting its scope.

### Example 1

This example demonstrates that FLT3 is expressed on acute lymphoblastic and acute myeloid leukemia cell lines.

The number of FLT3 receptors per cell is quantified on various acute lymphoblastic [NALM6 (DSMZ no. ACC 128), HB11;19 (Horsley et al., Genes Chromosomes Cancer, 45:554-564 (2006)), KOPN-8 (DSMZ no. ACC 552), SEM (DSMZ no. ACC 546)] and acute myeloid [MOLM13 (DSMZ no. ACC 554), MOLM14 (DSMZ no. ACC 577), MV4;11 (DSMZ no. ACC 102), THP-1 (DSMZ no. ACC 16)] leukemia cell lines by flow cytometry using BD Quantibrite beads as per manufacturer's protocol (BD Biosciences; San Jose. Calif. USA) and Phycoerythrin (PE) labeled anti human CD135 (FLT3) antibody (eBioscience; San Diego, Calif., USA; clone BV10A4H2), KOPN-8 is a cell line derived from a patient with infant ALL. DSMZ is the Leibniz-institute DSMZ-German Collection of Microorganisms and Cell Cultures (Braunschweig, Germany).

All cell lines are cultured using RPMI 1640 (Invitrogen; Carlsbad, Calif. USA) media supplemented with 10% heat inactivated fetal bovine serum (Omega Scientific; Tarzana, Calif., USA), 100 U/mL penicillin, 100 mg mL streptomycin, and 2 mM L-glutamine (Invitrogen). Labeled samples are analyzed by flow cytometry on a BD LSR Fortessa (BD Biosciences) and data analysis is performed using FlowJo software (FlowJo LLC, Ashland, Oreg., USA) and GraphPad Prism (GraphPad Software; La Jolla, Calif., USA).

### Example 2

This example demonstrates the production of a bispecific CAR in accordance with embodiments of the disclosure.

The amino acid sequences encoding the FLT3 and CD19 scFvs are converted to DNA sequences and codon optimized and synthesized using GeneArt™ gene synthesis (ThermoFisher Scientific; Waltham, Mass., USA) with kozak sequence, and membrane localization leader sequence from human granulocyte macrophage colony stimulating factor (GM-CSF). The scFv sequences are then subcloned from GeneArt™ vectors and moved using standard molecular cloning techniques to the third generation lentiviral plasmid pELNS-19BBzeta which contains the CD8 hinge and transmembrane, 4-1BB signaling domain, and the CD3zeta domain. The leader sequence is initially encoded and enhances trafficking to the cell surface; it is likely to be cleaved off in the mature form.

Lentiviral supernatant generation: 293T cells (ATCC; Manassas, Va., USA; ATCC no, CRL-3216) are transiently transfected with third generation lentiviral plasmids to generate viral supernatant, 293T cells are plated in poly-D lysine coated 15 cm tissue culture plates (Corning; Tewksbury, Mass., USA) in DMEM supplemented with 10% heat inactivated fetal bovine serum (Omega Scientific), 100 U/mL penicillin, 100 mg/mL streptomycin, and 2 mM L-glutamine (Invitrogen) and allowed to adhere for 16 hours. The following day, GFP or FLT3 CD19 bispecific CAR containing plasmids, pMDLg/pRRE and pRSV-Rev packaging, and pMD-G envelope plasmids are lipid transfected into the 293 T cells using Lipofectamine 3000 (Invitrogen) as per manufacturer's protocol. Media containing the transfection mixture is discarded and replaced with fresh media 4-6 hours after transfection mixture is added. Viral supernatant is collected at 24, 48, and 72 hours post transfection, centrifuged at 1200 rpm for 6 minutes to remove cells, and stored at −80° C. until use.

T cell source: Human elutriated lymphocytes from normal donors are used as a source of T cells for experiments. Donor lymphocytes are cleared of red blood cells using Lymphocyte Separation Medium (Lonza; Basel, Switzerland) as per manufacturer's protocol and cryopreserved in heat inactivated fetal bovine serum (FBS; Omega Scientific) with 10% Dimethyl sulfoxide (DMSO; Sigma Aldrich; St Louis, Mo., USA) and stored in liquid nitrogen.

T cell transduction: Elutriated lymphocytes are thawed and cultured in T cell expansion media (TCEM) which consists of AIM-V media (Invitrogen) supplemented with 5% heat inactivated FBS (Omega Scientific), 100 U/mL penicillin, 100 mg/mL streptomycin, 15 mM HEPES, and 2 mM L-glutamine (Invitrogen) and 40 IU/mL IL-2 with Dynabeads Human T-Expander CD3/CD28 beads (Invitrogen) at a 3:1 bead to cell ratio. Cells are cultured for 2 days prior to transduction with viral supernatant. Two million T cells are plated per well of a 6 well plate in 1 mL TCEM+3 mL viral supernatant with a final concentration of 40 IU/mL of IL-2 and 10 mg/mL of protamine sulfate. The 6-well plates of T cells are centrifuged at 872 g for 2 hours at 32° C. and then incubated at 37° C. overnight. The following day, Dynabeads are removed using a magnetic rack and the T cells are cultured in fresh TCEM with 300 IU/mL IL-2 at 500,000 cells/mL. T cells are cultured until day 9 in TCEM with 300 IU/mL of IL-2 maintaining the cells below 1 million/mL and the T cell transduction is determined by flow cytometry.

### Example 3

This example demonstrates FLT3 CD19 bispecific CAR T cell transduction in accordance with embodiments of the disclosure.

The CAR is a bispecific FLT3 CD19 CAR, preferably in some embodiments depicted in FIG. 1. The leader sequence is initially encoded and enhances trafficking to the cell surface; it is likely to be cleaved off in the mature form. The transduction efficiency of GFP and FLT3 CD19 bispecific CAR transduced T cells are determined on day 9 of T cell culture. GFP transduced T cells are analyzed for GFP positivity by flow cytometry using a LSR Fortessa (BD Biosciences). FLT3 CD19 bispecific CAR expression is determined using biotinylated protein L (Genscript; Piscataway, N.J., USA) which is a bacterial protein that binds to a subset of kappa light chains of antibodies. The NC7 based FLT3 CD19 bispecific CAR is a sequence that binds to protein L and the CAR expression can be determined by staining with streptavidin PE. As a negative control, FLT3 CD19 bispecific CAR T cells are stained with secondary streptavidin PE only. Data analysis is performed using FlowJo software (Flow Jo LLC).

### Example 4

This example demonstrates that T cells expressing FLT3 CD19 bispecific CARs secrete high levels of cytokines when co-cultured with FLT3 and/or CD19-expressing cell lines in accordance with embodiments of the disclosure. T cells expressing monospecific FLT3 CARs and monospecific CD19 CARs fail to secrete cytokines upon co-culture with CD19-FLT3+ target cells and CD19+FLT3−target cells respectively.

Bispecific CAR-expressing T cells are activated for cytokine secretion (Interferon gamma (IFN-g, IFN-γ)) upon stimulation by CD19+FLT3−, CD19−FLT3+, and CD19+FLT3+ target cells, which include CD19+FLT3−H9 cells, CD19−FLT3+H9 cells, CD19+FLT3+H9 cells and SUP-B15 cells. IFN-γ content is measured by cytokine bead array of culture supernatants of T cells and target cells after 24-hours of co-culture. Activated bispecific CAR-expressing T cells secrete IFN-γ upon stimulation by every type of target cell. In contrast, monospecific CAR expressing T cell lines are not activated for cytokine IFN-γ secretion upon stimulation by antigen-negative antigen loss escape variants, which escape from the monospecific CAR effector cells. CD19 CAR T cells fail to secrete IFN-γ upon co-culture with CD19−FLT3+ target cells and FLT3 CAR T cells fail to secrete IFN-γ upon co-culture with CD19+FLT3−target cells.

### Example 5

This example demonstrates that T cells expressing FLT3 CD19 bispecific CARs are able to lyse CD19+FLT3−, CD19−FLT3+, and CD19+FLT3+ target cells in accordance with embodiments of the disclosure. Monospecific CAR T cells were not able to lyse cells in each population.

A 4-hour chromium release assay is used to measure the lysis of target cells by effector cells. Effector cells are primary human T cells lentivirally transduced to express monospecific anti-CD19 CAR, monospecific anti-FLT3 CAR or bispecific anti-CD19×FLT3 CAR. The bispecific anti-CD19×FLT3 CAR effector T-cells effectively lyse all CD19+FLT3−, CD19−FLT3+, and CD19+FLT3+ target cells, which include CD19+FLT3−H9 cells, CD19−FLT3+H9 cells, CD19+FLT3+H9 cells and SUP-B15 cells.

In contrast, monospecific CAR expressing T cell lines fail to lyse antigen-negative antigen loss escape variants, which escape from the monospecific CAR effector cells. The anti-CD19 CAR effector T-cells fail to lyse CD19−FLT3+ targets and the anti-FLT3 CAR effector T-cells failed to lyse CD19+FLT3−targets.

All references, including publications, patent applications, and patents, cited herein are hereby incorporated by reference to the same extent as if each reference were individually and specifically indicated to be incorporated by reference and were set forth in its entirety herein.

The use of the terms “a” and “an’ and “‘the” and “at least one” and similar referents in the context of describing the disclosure (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean, one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

Preferred embodiments of this disclosure are described herein, including the best mode known to the inventors for carrying out the disclosure. Variations of those preferred embodiments may become apparent to those of ordinary skill in the art upon reading the foregoing description. The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

What is claimed is:

1. A nucleic acid molecule encoding a chimeric antigen receptor (CAR) comprising:
(a) a sequence encoding an antigen binding domain specific for FLT3,
(b) a sequence encoding an antigen binding domain specific for a second antigen,
(c) a sequence encoding a spacer,
(d) a sequence encoding a transmembrane domain, and
(e) a sequence encoding an intracellular T cell signaling domain.

2. The nucleic acid molecule of claim 1, wherein the nucleic acid molecule further comprises a sequence encoding a linker.

3. The nucleic acid molecule of claim 1 or 2, wherein the sequence encoding a linker is positioned between the sequence encoding an antigen binding domain specific for FLT3 and the sequence encoding an antigen binding domain specific for a second antigen.

4. The nucleic acid molecule of any one of claims 1-3, wherein the second antigen is selected from the group consisting of CD antigen 19 (CD19), CD antigen 22 (CD22), CD antigen 33 (CD33), CD antigen 123 (CD123), Lewis-Y, CD antigen 44, isoform 6 (CD44v6), C-type lectin-like molecule 1 (CLL-1), folate receptor-beta (FOLR2), CD antigen 13 (CD13), CD antigen 15 (CD15), CD antigen 30 (CD30), CD antigen 45 (CD45), CD antigen 47 (CD47), Angiopoietin-2 (Ang-2) CD antigen 133 (CD133), Wilms tumor protein (WT1), Vascular endothelial growth factor A (VEGF-A), U5 small nuclear ribonucleoprotein 200 kDa helicase (U5 snRNP200), Adhesion G protein-coupled receptor E2 (ADGRE2), CD antigen 38 (CD38), CD antigen 157 (CD157), Leukocyte immunoglobulin-like receptor subfamily B member 2 (LILRB2), C-C chemokine receptor type 1 (CCR-1), Proto-oncogene C-KIT, and CD antigen 43 (CD43).

5. The nucleic acid molecule of any one of claims 1-4, further comprising a sequence encoding at least one costimulatory domain.

6. The nucleic acid molecule of claim 5, wherein the at least one costimulatory domain comprises a sequence from or a sequence derived from CD2, CD3 delta (CD3δ), CD3 epsilon (CD3ε), CD3 gamma (CD3γ), CD4, CD7, CD8α, CD8β, CD28, CD40, CD137 (4-IBB), CD247 (CD3-zeta (CD3ζ)), CD276 (B7-H3), CD279 (PD-1), IL-2R beta (IL-2β), IL-2R gamma (IL-2Rγ), IL-7R alpha (IL-7Rα), CTLa4, inducible T cell co-stimulator (ICOS), lymphocyte function-associated antigen-1 LFA-1 (CD 11α/CD18), ICAM-1, a CD83 ligand, a Fc gamma receptor, MHC class 1 molecule, MHC class 2 molecule, a TNF receptor protein, an immunoglobulin protein, a cytokine receptor, or an integrin.

7. The nucleic acid molecule of any one of claims 1-6, wherein the antigen binding domain specific for FLT3 comprises one or more of the sequences of SEQ ID NO: 6, SEQ ID NO: 8, SEQ ID NO: 10, SEQ ID NO: 14, SEQ ID NO: 16, and SEQ ID NO: 18.

8. A nucleic acid molecule comprising:
(a) a sequence encoding a first CAR comprising a sequence encoding an antigen binding domain with specificity for FLT3 and a sequence encoding an intracellular T cell signaling domain, and
(b) a sequence encoding a second CAR comprising a sequence encoding an antigen binding domain with specificity for a second antigen and a sequence encoding an intracellular T cell signaling domain.

9. The nucleic acid molecule of claim 8, wherein the sequence encoding a first CAR comprises:
(a) a sequence encoding an antigen binding domain specific for FLT3,
(b) a sequence encoding a spacer,
(c) a sequence encoding a transmembrane domain, and
(d) a sequence encoding an intracellular T cell signaling domain.

10. The nucleic acid molecule of claim 8 or 9, wherein the sequence encoding a second CAR comprises:
(a) a sequence encoding an antigen binding domain specific for the second antigen,
(b) a sequence encoding a spacer,
(c) a sequence encoding a transmembrane domain, and
(d) a sequence encoding an intracellular T cell signaling domain.

11. The nucleic acid molecule of any one of claims 8-10, wherein the nucleic acid molecule further comprises a sequence encoding a cleavable domain.

12. The nucleic acid molecule of claim 11, wherein the sequence encoding a cleavable domain is positioned between the sequence encoding the first CAR and the sequence encoding the second CAR.

13. The nucleic acid molecule of claim 11 or 12, wherein the cleavable domain comprises a sequence encoding a 2A self-cleaving peptide.

14. The nucleic acid molecule of claim 13, wherein the 2A self-cleaving peptide comprises the sequence of GDVEXNPGP or a nucleic acid sequence encoding the amino acid sequence of GDVEXNPGP.

15. The nucleic acid molecule of claim 13, wherein the sequence encoding a 2A self-cleaving peptide comprises the sequence of a P2A self-cleaving peptide, a T2A self-cleaving peptide, a E2A self-cleaving peptide, a F2A self-cleaving peptide, a BmCPV2A self-cleaving peptide, or a BmIFV2A self-cleaving peptide.

16. The nucleic acid molecule of any one of claims 8-15, wherein the second antigen is selected from the group consisting of CD antigen 19 (CD19), CD antigen 22 (CD22), CD antigen 33 (CD33), CD antigen 123 (CD123), Lewis-Y, CD antigen 44, isoform 6 (CD44v6), C-type lectin-like molecule 1 (CLL-1), folate receptor-beta (FOLR2), CD antigen 13 (CD13), CD antigen 15 (CD15), CD antigen 30 (CD30), CD antigen 45 (CD45), CD antigen 47 (CD47), Angiopoietin-2 (Ang-2) CD antigen 133 (CD133), Wilms tumor protein (WT1), Vascular endothelial growth factor A (VEGF-A), U5 small nuclear ribonucleoprotein 200 kDa helicase (U5 snRNP200), Adhesion G protein-coupled receptor E2 (ADGRE2), CD antigen 38 (CD38), CD antigen 157 (CD157), Leukocyte immunoglobulin-like receptor subfamily B member 2 (LILRB2), C-C chemokine receptor type 1 (CCR-1), Proto-oncogene C-KIT, and CD antigen 43 (CD43).

17. The nucleic acid molecule of any one of claims 8-16, wherein the sequence encoding the first CAR further comprises a sequence encoding at least one costimulatory domain and wherein the sequence encoding the second CAR further comprises a sequence encoding at least one costimulatory domain.

18. The nucleic acid molecule of claim 17, wherein the at least one costimulatory domain comprises a sequence from or a sequence derived from CD2, CD3 delta (CD3δ), CD3 epsilon (CD3ε), CD3 gamma (CD3γ), CD4, CD7, CD8α, CD8β, CD28, CD40, CD137 (4-IBB), CD247 (CD3-zeta (CD3ζ)), CD276 (B7-H3), CD279 (PD-1), IL-2R beta (IL-2β), IL-2R gamma (IL-2Rγ), IL-7R alpha (IL-7Rα), CTLa4, inducible T cell co-stimulator (ICOS), lymphocyte function-associated antigen-1 LFA-1 (CD 11α/CD18), ICAM-1, a CD83 ligand, a Fc gamma receptor, MHC class 1 molecule, MHC class 2 molecule, a TNF receptor protein, an immunoglobulin protein, a cytokine receptor, or an integrin.

19. The nucleic acid molecule of any one of claims 8-18, wherein the antigen binding domain specific for FLT3 comprises one or more of the sequences of SEQ ID NO: 6, SEQ ID NO: 8, SEQ ID NO: 10, SEQ ID NO: 14, SEQ ID NO: 16, and SEQ ID NO: 18.

20. The nucleic acid molecule of any one of claims 8-19, wherein the sequence encoding a first CAR comprises a codon-optimized sequence.

21. The nucleic acid molecule of any one of claims 8-20, wherein the sequence encoding a second CAR comprises a codon-optimized sequence.

22. The nucleic acid molecule of any one of claims 8-21, wherein the codon-optimized sequence encoding a first CAR and the codon-optimized sequence encoding a second CAR are codon-optimized to reduce recombination between the sequence encoding the first CAR and the sequence encoding the second CAR.

23. A composition comprising the nucleic acid molecule of any one of claims 1-22.

24. The composition of claim 23, further comprising a pharmaceutically acceptable carrier.

25. A vector comprising the nucleic acid of any one of claims 1-22.

26. The vector of claim 25, wherein the vector is a recombinant vector.

27. The vector of claim 25 or 26, wherein the vector is an expression vector.

28. The vector of any one of claims 25-27, wherein the vector is a non-viral vector.

29. The vector of any one of claims 25-27, wherein the vector is a viral vector.

30. The vector of claim 29, wherein the viral vector is a retroviral vector.

31. The vector of claim 29 or 30, wherein the viral vector is a lentiviral vector.

32. The vector of any one of claims 25-31, wherein the vector is formulated for administration to an immune cell.

33. The vector of any one of claims 25-32, wherein the vector is formulated for administration to a T cell.

34. A composition comprising a vector of any one of claims 25-33.

35. The composition of claim 34, further comprising a pharmaceutically-acceptable carrier.

36. A cell comprising the nucleic acid molecule of any one of claims 1-22.

37. A cell comprising the vector of any one of claims 25-33.

38. A cell comprising the composition of any one of claim 23-24 or 34-35.

39. The cell of any one of claims 36-38, wherein the cell is an immune cell

40. The cell of any one of claims 36-39, wherein the cell is a T cell.

41. The cell of any one of claims 36-39, wherein the cell is a NK cell.

42. A composition comprising the cell of any one of claims 36-41.

43. The composition of claim 42, further comprising pharmaceutically acceptable carrier.

44. A composition comprising a first cell and a second cell, wherein the first cell comprises a first nucleic acid molecule and the second cell comprises a second nucleic acid molecule, wherein the first nucleic acid molecule encodes a first CAR comprising an antigen binding domain with specificity for FLT3 and an intracellular T cell signaling domain, and wherein the second nucleic acid molecule encodes a second CAR comprising an antigen binding domain with specificity for a second antigen and an intracellular T cell signaling domain.

45. The composition of claim 44, wherein the sequence encoding a first CAR comprises:
(a) a sequence encoding an antigen binding domain specific for FLT3,
(b) a sequence encoding a spacer,
(c) a sequence encoding a transmembrane domain, and
(d) a sequence encoding an intracellular T cell signaling domain.

46. The composition of claim 44 or 45, wherein the sequence encoding a second CAR comprises:
(a) a sequence encoding an antigen binding domain specific for the second antigen,
(b) a sequence encoding a spacer,
(c) a sequence encoding a transmembrane domain, and
(d) a sequence encoding an intracellular T cell signaling domain.

47. The composition of any one of claims 44-46, wherein the second antigen is selected from the group consisting of CD antigen 19 (CD19), CD antigen 22 (CD22), CD antigen 33 (CD33), CD antigen 123 (CD123), Lewis-Y, CD antigen 44, isoform 6 (CD44v6), C-type lectin-like molecule 1 (CLL-1), folate receptor-beta (FOLR2), CD antigen 13 (CD13), CD antigen 15 (CD15), CD antigen 30 (CD30), CD antigen 45 (CD45), CD antigen 47 (CD47), Angiopoietin-2 (Ang-2) CD antigen 133 (CD133), Wilms tumor protein (WT1), Vascular endothelial growth factor A (VEGF-A), U5 small nuclear ribonucleoprotein 200 kDa helicase (U5 snRNP200), Adhesion G protein-coupled receptor E2 (ADGRE2), CD antigen 38 (CD38), CD antigen 157 (CD157), Leukocyte immunoglobulin-like receptor subfamily B member 2 (LILRB2), C-C chemokine receptor type 1 (CCR-1), Proto-oncogene C-KIT, and CD antigen 43 (CD43).

48. The composition of any one of claims 44-47, wherein the first CAR further comprises at least one costimulatory domain, and wherein the second CAR further comprises at least one costimulatory domain.

49. The composition of claim 48, wherein the at least one costimulatory domain comprises a sequence from or a sequence derived from CD2, CD3 delta (CD3δ), CD3 epsilon (CD3ε), CD3 gamma (CD3γ), CD4, CD7, CD8α, CD8β, CD28, CD40, CD137 (4-IBB), CD247 (CD3-zeta (CD3ζ)), CD276 (B7-H3), CD279 (PD-1), IL-2R beta (IL-2β), IL-2R gamma (IL-2Rγ), IL-7R alpha (IL-7Rα), CTLa4, inducible T cell co-stimulator (ICOS), lymphocyte function-associated antigen-1 LFA-1 (CD 11α/CD18), ICAM-1, a CD83 ligand, a Fc gamma receptor, MHC class 1 molecule, MHC class 2 molecule, a TNF receptor protein, an immunoglobulin protein, a cytokine receptor, or an integrin.

50. The composition of any one of claim 23-24, 34-35 or 42-49 for use in the treatment of cancer.

51. The composition of any one of claim 23-24, 34-35 or 42-49 for use in the treatment of acute lymphocytic leukemia (ALL).

52. The composition of any one of claim 23-24, 34-35 or 42-49 for use in the treatment of acute myeloid leukemia (AML).

53. A method for treating cancer comprising: administering a therapeutically effective amount of the composition of any one of claim 23-24, 34-35 or 42-49 to a subject, wherein the cancer expresses FLT3.

54. The method of claim 53, wherein the cancer further expresses a second antigen selected from the group consisting of CD antigen 19 (CD19), CD antigen 22 (CD22), CD antigen 33 (CD33), CD antigen 123 (CD123), Lewis-Y, CD antigen 44, isoform 6 (CD44v6), C-type lectin-like molecule 1 (CLL-1), folate receptor-beta (FOLR2), CD antigen 13 (CD13), CD antigen 15 (CD15), CD antigen 30 (CD30), CD antigen 45 (CD45), CD antigen 47 (CD47), Angiopoietin-2 (Ang-2) CD antigen 133 (CD133), Wilms tumor protein (WT1), Vascular endothelial growth factor A (VEGF-A), U5 small nuclear ribonucleoprotein 200 kDa helicase (U5 snRNP200), Adhesion G protein-coupled receptor E2 (ADGRE2), CD antigen 38 (CD38), CD antigen 157 (CD157), Leukocyte immunoglobulin-like receptor subfamily B member 2 (LILRB2), C-C chemokine receptor type 1 (CCR-1), Proto-oncogene C-KIT, and CD antigen 43 (CD43).

55. The method of claim 53 or 54, wherein the cancer is refractory to radiation, small molecule or biologic chemotherapeutic intervention.
